SEQUENCE LISTING (163 sequences)

SEQ ID NO: 1 - protein, 119 aa, synthetic construct
  REGION 1..119: note = heavy chain variable region
EVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSS

SEQ ID NO: 2 - protein, 30 aa, synthetic construct
  REGION 1..30: note = heavy chain FR1
EVQLLESGGGLVQPGGSLRLSCAASGFTVT

SEQ ID NO: 3 - protein, 5 aa, synthetic construct
  REGION 1..5: note = anti-CD122 HCDR1
SYAVH

SEQ ID NO: 4 - protein, 14 aa, synthetic construct
  REGION 1..14: note = heavy chain FR2
WVRQAPGKGLEWLG

SEQ ID NO: 5 - protein, 16 aa, Mus musculus
VIWSGGSTDYNAAFIS

SEQ ID NO: 6 - protein, 32 aa, synthetic construct
  REGION 1..32: note = heavy chain FR3
RLTISKDNSKNTVYFQMNSLRAEDTAVYYCAR

SEQ ID NO: 7 - protein, 11 aa, synthetic construct
  REGION 1..11: note = HCDR3
AGDANYDGFAY

SEQ ID NO: 8 - protein, 11 aa, synthetic construct
  REGION 1..11: note = heavy chain FR4
WGQGTLVTVSS

SEQ ID NO: 9 - protein, 106 aa, synthetic construct
  REGION 1..106: note = light chain variable region
DIQMTQSPSSLSASVGDRVTITCQASQSVSFLYWYQQRPGKAPRLLIYDTSNLASGVPSRFSGSGSGTSYTFTISSLQPEDIATYYCQQWSTYPLTFGQGTKVEIK

SEQ ID NO: 10 - protein, 23 aa, synthetic construct
  REGION 1..23: note = light chain FR1
DIQMTQSPSSLSASVGDRVTITC

SEQ ID NO: 11 - protein, 10 aa, synthetic construct
  REGION 1..10: note = LCDR1
QASQSVSFLY

SEQ ID NO: 12 - protein, 15 aa, synthetic construct
  REGION 1..15: note = light chain FR2
WYQQRPGKAPRLLIY

SEQ ID NO: 13 - protein, 7 aa, Mus musculus
DTSNLAS

SEQ ID NO: 14 - protein, 32 aa, synthetic construct
  REGION 1..32: note = light chain FR3
GVPSRFSGSGSGTSYTFTISSLQPEDIATYYC

SEQ ID NO: 15 - protein, 9 aa, Mus musculus
QQWSTYPLT

SEQ ID NO: 16 - protein, 10 aa, synthetic construct
  REGION 1..10: note = light chain FR4
FGQGTKVEIK

SEQ ID NO: 17 - protein, 106 aa, synthetic construct
  REGION 1..106: note = light chain variable region
DIQMTQSPSSLSASVGDRVTITCQASSSVSFMYWYQQRPGKAPRLLIYDTSNLASGVPSRFSGSGSGTSYTFTISSLQPEDIATYYCQQWSTYPLTFGQGTKVEIK

SEQ ID NO: 18 - protein, 10 aa, synthetic construct
  REGION 1..10: note = LCDR1
QASSSVSFMY

SEQ ID NO: 19 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 20 - protein, 551 aa, Macaca fascicularis
MATLALSWCLPLLILLLPLATSSASAAVNGTSRFTCFYNSRANISCVWSQDGALQDTSCQVHAWPDRRRWNQTCELLPVSQASWACNLILGTPDSQKLTAVDIVTLRVMCREGVRWRMMAIQDFKPFENLRLMAPISLQVVHVETHRCNISWKISQASHYFERHLEFEARTLSPGHTWEEAPLMTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCHTPDPSKFFSQLTSEHGGDVQKWLSSPFPSSSFSPGGLAPEISPLEVLERDKVTQLLLQQDKVPEPSSLSSNRSLTSCFTNQGYFFFHLPDALEIEACQVYFTYDPCAEEEPDEGGADAPTGSSPQPLRPLSAEDDAYCTFPSGDDLLLFSPSLLGGPSPPSTAPGGSGAGEERLPPSLQERVPRDWDPQPLGPPTPGVPDLVDFQPRPELVLREAGEQVPDPGPREPFSFPWARPPGQGEVRALNARLPLNTDAYLSLQELQDQDPTHLV

SEQ ID NO: 21 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 22 - protein, 119 aa, synthetic construct
  REGION 1..119: note = heavy chain variable region
EVQLLESGGGLVQPGGSLRLSCAASGFSVTSYGVHWIRQAPGKGLEWLGVIWSGGSTDYNAAFISRLTISKDNSKNTVYFQMNSLQAEDTAIYYCARAGDYNYDGFAYWGQGTLVTVSS

SEQ ID NO: 23 - protein, 30 aa, synthetic construct
  REGION 1..30: note = heavy chain FR1
EVQLLESGGGLVQPGGSLRLSCAASGFSVT

SEQ ID NO: 24 - protein, 5 aa, Mus musculus
SYGVH

SEQ ID NO: 25 - protein, 14 aa, synthetic construct
  REGION 1..14: note = heavy chain FR2
WIRQAPGKGLEWLG

SEQ ID NO: 26 - protein, 32 aa, synthetic construct
  REGION 1..32: note = heavy chain FR3
RLTISKDNSKNTVYFQMNSLQAEDTAIYYCAR

SEQ ID NO: 27 - protein, 11 aa, Mus musculus
AGDYNYDGFAY

SEQ ID NO: 28 - protein, 106 aa, synthetic construct
  REGION 1..106: note = light chain variable region
DIVLTQSPSSLSASVGDRVTITCSGSSSVSFMYWYQQRPGKAPRLLIYDTSNLASGVPSRFSGSGSGTSYTFTISSLQPEDIATYYCQQWSTYPLTFGQGTKVEVK

SEQ ID NO: 29 - protein, 23 aa, synthetic construct
  REGION 1..23: note = light chain FR1
DIVLTQSPSSLSASVGDRVTITC

SEQ ID NO: 30 - protein, 10 aa, Mus musculus
SGSSSVSFMY

SEQ ID NO: 31 - protein, 10 aa, synthetic construct
  REGION 1..10: note = light chain FR4
FGQGTKVEVK

SEQ ID NO: 32 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 327 aa, synthetic construct
  REGION 1..327: note = IgG4 (S228P)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 330 aa, Homo sapiens [sequence omitted]

SEQ ID NO: 35 - protein, 330 aa, synthetic construct
  REGION 1..330: note = IgG1-3M
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 37 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 10 aa, synthetic construct
  REGION 1..10: note = HCDR1
  VARIANT 8: note = L or any other amino acid
GFTFSSYXMS

SEQ ID NO: 40 - protein, 19 aa, synthetic construct
  REGION 1..19: note = HCDR2
  VARIANT 1: note = V or a conservative substitution of V
  VARIANT 3: note = T or N
  VARIANT 9: note = A or S
  VARIANT 10: note = E or N
  VARIANT 11: note = T or K
  VARIANT 14: note = P or V
XAXISGGGXXXYYXDSVKG

SEQ ID NO: 41 - protein, 119 aa, synthetic construct
  REGION 1..119: note = MAB01-MAB15 IgG1 Designer variable domain sequence DVH1
EVQLLESGGGLVQPGGSLRLSCAASGFSVSSYGMHWVRQAPGKGLEWLGAIWSGGSTDYADAVKGRFTISRDNSKNTLYLQMNSLRAEDTAVYYCARAGDKNYDGFAYWGQGTLVTVSS

SEQ ID NO: 42 - protein, 119 aa, synthetic construct
  REGION 1..119: note = MAB01-MAB15 IgG1 Designer variable domain sequence DVH2
EVQLLESGGGLVQPGGSLRLSCAASGFSVSSYGMHWVRQAPGKGLEWLGVIWSGGSTDYADAVKGRFTISRDNSKNTLYLQMNSLRAEDTAVYYCARAGDKNYDGFAYWGQGTLVTVSS

SEQ ID NO: 43 - protein, 119 aa, synthetic construct
  REGION 1..119: note = MAB01-MAB15 IgG1 Designer variable domain sequence DVH3
EVQLLESGGGLVQPGGSLRLSCAASGFSVSSYGMHWVRQAPGKGLEWLGAIWSGGSTDYADAVKGRFTISRDNSKNTLYLQMNSLRAEDTAVYYCARAGDHNYDGFAYWGQGTLVTVSS

SEQ ID NO: 44 - protein, 119 aa, synthetic construct
  REGION 1..119: note = MAB01-MAB15 IgG1 Designer variable domain sequence DVH4
EVQLLESGGGLVQPGGSLRLSCAASGFSVSSYGMHWVRQAPGKGLEWLGVIWSGGSTDYADAVKGRFTISRDNSKNTLYLQMNSLRAEDTAVYYCARAGDHNYDGFAYWGQGTLVTVSS

SEQ ID NO: 45 - protein, 119 aa, synthetic construct
  REGION 1..119: note = MAB01-MAB15 IgG1 Designer variable domain sequence DVH5
EVQLLESGGGLVQPGGSLRLSCAASGFSVSSYGMHWVRQAPGKGLEWLGAIWSGGSTDYNDAVKGRFTISRDNSKNTLYLQMNSLRAEDTAVYYCARAGDHNYDGFAYWGQGTLVTVSS

SEQ ID NO: 46 - protein, 106 aa, synthetic construct
  REGION 1..106: note = MAB01-MAB15 IgG1 Designer variable domain sequence DVL1
DIQMTQSPSSLSASVGDRVTITCQASQSISYMYWYQQRPGKAPKLLIYDTSNLATGVPSRFSGSGSGTSYTFTISSLQPEDIATYYCQQWDNYPLTFGGGTKVEIK

SEQ ID NO: 47 - protein, 106 aa, synthetic construct
  REGION 1..106: note = MAB01-MAB15 IgG1 Designer variable domain sequence DVL2
DIQMTQSPSSLSASVGDRVTITCQASQDISYLYWYQQRPGKAPKLLIYDASNLATGVPSRFSGSGSGTSYTFTISSLQPEDIATYYCQQWDNYPLTFGGGTKVEIK

SEQ ID NO: 48 - protein, 106 aa, synthetic construct
  REGION 1..106: note = MAB01-MAB15 IgG1 Designer variable domain sequence DVL3
DIQMTQSPSSLSASVGDRVTITCQASQDISYLYWYQQRPGKAPKLLIYDASNLETGVPSRFSGSGSGTSYTFTISSLQPEDIATYYCQQWDNYPLTFGGGTKVEIK

SEQ ID NO: 49 - protein, 119 aa, synthetic construct
  REGION 1..119: note = 06F11-V optimization variable domain VH sequence F11VH-1
EVQLLESGGGLVQPGGSLRLSCAASGFSVTSYGVHWIRQAPGKGLEWLGAIWSGGSTQYNAAVKGRFTISRDNSKNTLYLQMNSLRAEDTAVYYCARAGDANYDGFAYWGQGTLVTVSS

SEQ ID NO: 50 - protein, 119 aa, synthetic construct
  REGION 1..119: note = 06F11-V optimization variable domain VH sequence F11VH-13
EVQLLESGGGLVQPGGSLRLSCAASGFSVTSYGVHWIRQAPGKGLEWLGAIWSGGSTQYNAAVKGRFTISRDNSKNTLYLQMNSLRAEDTAVYYCARAGDYNYDGFAYWGQGTLVTVSS

SEQ ID NO: 51 - protein, 119 aa, synthetic construct
  REGION 1..119: note = 06F11-V optimization variable domain VH sequence F11VH-23
EVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDYNYDGFAYWGQGTLVTVSS

SEQ ID NO: 52 - protein, 119 aa, synthetic construct
  REGION 1..119: note = 06F11-V optimization variable domain VH sequence F11VH-12
EVQLLESGGGLVQPGGSLRLSCAASGFSVTSYGVHWIRQAPGKGLEWLGVIWSGGSTDYNAAFISRLTISKDNSKNTVYFQMNSLRAEDTAVYYCARAGDANYDGFAYWGQGTLVTVSS

SEQ ID NO: 53 - protein, 119 aa, synthetic construct
  REGION 1..119: note = 06F11-V optimization variable domain VH sequence F11VH-123
EVQLLESGGGLVQPGGSLRLSCAASGFSVTSYGVHWIRQAPGKGLEWLGVIWSGGSTDYNAAFISRLTISKDNSKNTVYFQMNSLRAEDTAVYYCARAGDYNYDGFAYWGQGTLVTVSS

SEQ ID NO: 54 - protein, 106 aa, synthetic construct
  REGION 1..106: note = 06F11-V optimization variable domain VK sequence F11VK-12
DIQMTQSPSSLSASVGDRVTITCQASSSVSFMYWYQQRPGKAPRLLIYDTSNLASGVPSRFSGSGSGTSYTFTISSLQPEDIATYYCQQWDTYPLTFGGGTKVEIK

SEQ ID NO: 55 - protein, 106 aa, synthetic construct
  REGION 1..106: note = 06F11-V optimization variable domain VK sequence F11VK-123
DIQMTQSPSSLSASVGDRVTITCQASSSVSFMYWYQQRPGKAPRLLIYDTSNLASGVPSRFSGSGSGTSYTFTISSLQPEDIATYYCQQWDTYPLTFGQGTKVEIK

SEQ ID NO: 56 - protein, 5 aa, synthetic construct
  REGION 1..5: note = HCDR1
SYAMH

SEQ ID NO: 57 - protein, 5 aa, synthetic construct
  REGION 1..5: note = HCDR1
SYGMH

SEQ ID NO: 58 - protein, 16 aa, synthetic construct
  REGION 1..16: note = HCDR2
AIWSGGSTDYAASVKG

SEQ ID NO: 59 - protein, 16 aa, synthetic construct
  REGION 1..16: note = HCDR2
AIWSGGSTDYADAVKG

SEQ ID NO: 60 - protein, 16 aa, synthetic construct
  REGION 1..16: note = HCDR2
VIWSGGSTDYADSVKG

SEQ ID NO: 61 - protein, 16 aa, synthetic construct
  REGION 1..16: note = HCDR2
VIWSGGSTDYADAVKG

SEQ ID NO: 62 - protein, 16 aa, synthetic construct
  REGION 1..16: note = HCDR2
VIWSGGSTDYNDAVKG

SEQ ID NO: 63 - protein, 16 aa, synthetic construct
  REGION 1..16: note = HCDR2
AIWSGGSTDYAAAVKG

SEQ ID NO: 64 - protein, 16 aa, synthetic construct
  REGION 1..16: note = HCDR2
AIWSGGSTDYAATVKG

SEQ ID NO: 65 - protein, 16 aa, synthetic construct
  REGION 1..16: note = HCDR2
AIWSGGSTDYADSVKG

SEQ ID NO: 66 - protein, 16 aa, synthetic construct
  REGION 1..16: note = HCDR2
AIWSGGSTDYNAAVKG

SEQ ID NO: 67 - protein, 16 aa, synthetic construct
  REGION 1..16: note = HCDR2
AIWSGGSTDYNASVKG

SEQ ID NO: 68 - protein, 16 aa, synthetic construct
  REGION 1..16: note = HCDR2
AIWSGGSTDYNDAVKG

SEQ ID NO: 69 - protein, 16 aa, synthetic construct
  REGION 1..16: note = HCDR2
AIWSGGSTDYNDSVKG

SEQ ID NO: 70 - protein, 16 aa, synthetic construct
  REGION 1..16: note = HCDR2
AIWSGGSTQYNAAVKG

SEQ ID NO: 71 - protein, 16 aa, synthetic construct
  REGION 1..16: note = HCDR2
AIWSGGSTYYADAVKG

SEQ ID NO: 72 - protein, 16 aa, synthetic construct
  REGION 1..16: note = HCDR2
AIWSGGSTYYNAAVKG

SEQ ID NO: 73 - protein, 16 aa, synthetic construct
  REGION 1..16: note = HCDR2
AIWSGGSTYYNASVKG

SEQ ID NO: 74 - protein, 16 aa, synthetic construct
  REGION 1..16: note = HCDR2
AIWSGGSTYYNDAVKG

SEQ ID NO: 75 - protein, 16 aa, synthetic construct
  REGION 1..16: note = HCDR2
AIYSGGSTDYAAAVKG

SEQ ID NO: 76 - protein, 16 aa, synthetic construct
  REGION 1..16: note = HCDR2
AIYSGGSTDYADAVKG

SEQ ID NO: 77 - protein, 16 aa, synthetic construct
  REGION 1..16: note = HCDR2
AIYSGGSTDYNAAVKG

SEQ ID NO: 78 - protein, 16 aa, synthetic construct
  REGION 1..16: note = HCDR2
AIYSGGSTDYNDAVKG

SEQ ID NO: 79 - protein, 16 aa, synthetic construct
  REGION 1..16: note = HCDR2
AIYSGGSTYYAASVKG

SEQ ID NO: 80 - protein, 16 aa, synthetic construct
  REGION 1..16: note = HCDR2
AIYSGGSTYYADAVKG

SEQ ID NO: 81 - protein, 16 aa, synthetic construct
  REGION 1..16: note = HCDR2
AIYSGGSTYYNDAVKG

SEQ ID NO: 82 - protein, 16 aa, synthetic construct
  REGION 1..16: note = HCDR2
VIWSGGSTDYAAAVKG

SEQ ID NO: 83 - protein, 16 aa, synthetic construct
  REGION 1..16: note = HCDR2
VIWSGGSTDYAASVKG

SEQ ID NO: 84 - protein, 16 aa, synthetic construct
  REGION 1..16: note = HCDR2
VIWSGGSTDYNAAVKG

SEQ ID NO: 85 - protein, 16 aa, synthetic construct
  REGION 1..16: note = HCDR2
VIWSGGSTDYNASVKG

SEQ ID NO: 86 - protein, 16 aa, synthetic construct
  REGION 1..16: note = HCDR2
VIWSGGSTDYNDSVKG

SEQ ID NO: 87 - protein, 16 aa, synthetic construct
  REGION 1..16: note = HCDR2
VIWSGGSTYYNDAVKG

SEQ ID NO: 88 - protein, 16 aa, synthetic construct
  REGION 1..16: note = HCDR2
VIYSGGSTDYAASVKG

SEQ ID NO: 89 - protein, 16 aa, synthetic construct
  REGION 1..16: note = HCDR2
VIYSGGSTDYADSVKG

SEQ ID NO: 90 - protein, 16 aa, synthetic construct
  REGION 1..16: note = HCDR2
VIYSGGSTDYNDAVKG

SEQ ID NO: 91 - protein, 16 aa, synthetic construct
  REGION 1..16: note = HCDR2
VIYSGGSTYYNAAVKG

SEQ ID NO: 92 - protein, 11 aa, synthetic construct
  REGION 1..11: note = HCDR3
AGDYEYDGFAY

SEQ ID NO: 93 - protein, 11 aa, synthetic construct
  REGION 1..11: note = HCDR3
AGDKNYDGFAY

SEQ ID NO: 94 - protein, 11 aa, synthetic construct
  REGION 1..11: note = HCDR3
AGDQNYDGFAY

SEQ ID NO: 95 - protein, 11 aa, synthetic construct
  REGION 1..11: note = HCDR3
AGDENYDGFAY

SEQ ID NO: 96 - protein, 11 aa, synthetic construct
  REGION 1..11: note = HCDR3
AGDHNYDGFAY

SEQ ID NO: 97 - protein, 11 aa, synthetic construct
  REGION 1..11: note = HCDR3
AGDMNYDGFAY

SEQ ID NO: 98 - protein, 11 aa, synthetic construct
  REGION 1..11: note = HCDR3
AGDNNYDGFAY

SEQ ID NO: 99 - protein, 11 aa, synthetic construct
  REGION 1..11: note = HCDR3
AGDNNYDGFEY

SEQ ID NO: 100 - protein, 11 aa, synthetic construct
  REGION 1..11: note = HCDR3
AGDYDYDGFAY

SEQ ID NO: 101 - protein, 11 aa, synthetic construct
  REGION 1..11: note = HCDR3
AGDYNLDGFAY

SEQ ID NO: 102 - protein, 11 aa, synthetic construct
  REGION 1..11: note = HCDR3
AGDYNWDGFAY

SEQ ID NO: 103 - protein, 11 aa, synthetic construct
  REGION 1..11: note = HCDR3
AGDYNYDGFAI

SEQ ID NO: 104 - protein, 11 aa, synthetic construct
  REGION 1..11: note = HCDR3
AGDYNYDGFAM

SEQ ID NO: 105 - protein, 11 aa, synthetic construct
  REGION 1..11: note = HCDR3
AGDYNYDGFAN

SEQ ID NO: 106 - protein, 11 aa, synthetic construct
  REGION 1..11: note = HCDR3
AGDYNYDGFAW

SEQ ID NO: 107 - protein, 11 aa, synthetic construct
  REGION 1..11: note = HCDR3
AGDYNYDGFRY

SEQ ID NO: 108 - protein, 11 aa, synthetic construct
  REGION 1..11: note = HCDR3
AGDYNYDGLAY

SEQ ID NO: 109 - protein, 11 aa, synthetic construct
  REGION 1..11: note = HCDR3
AGNYNYDGFAY

SEQ ID NO: 110 - protein, 11 aa, synthetic construct
  REGION 1..11: note = HCDR3
AGPYNYDGFAY

SEQ ID NO: 111 - protein, 11 aa, synthetic construct
  REGION 1..11: note = HCDR3
AGTYNYDGFAY

SEQ ID NO: 112 - protein, 10 aa, synthetic construct
  REGION 1..10: note = LCDR1
QASQDISFMY

SEQ ID NO: 113 - protein, 10 aa, synthetic construct
  REGION 1..10: note = LCDR1
QASQSVSHLY

SEQ ID NO: 114 - protein, 10 aa, synthetic construct
  REGION 1..10: note = LCDR1
QASQSVSYMY

SEQ ID NO: 115 - protein, 10 aa, synthetic construct
  REGION 1..10: note = LCDR1
QASSSISFMY

SEQ ID NO: 116 - protein, 10 aa, synthetic construct
  REGION 1..10: note = LCDR1
QASSSISYMY

SEQ ID NO: 117 - protein, 10 aa, synthetic construct
  REGION 1..10: note = LCDR1
QASSSVSHMY

SEQ ID NO: 118 - protein, 10 aa, synthetic construct
  REGION 1..10: note = LCDR1
QASQSISFLY

SEQ ID NO: 119 - protein, 10 aa, synthetic construct
  REGION 1..10: note = LCDR1
QASQSISHLY

SEQ ID NO: 120 - protein, 10 aa, synthetic construct
  REGION 1..10: note = LCDR1
QASQSISHMY

SEQ ID NO: 121 - protein, 10 aa, synthetic construct
  REGION 1..10: note = LCDR1
QASSDISHLY

SEQ ID NO: 122 - protein, 10 aa, synthetic construct
  REGION 1..10: note = LCDR1
QASSSVSHLY

SEQ ID NO: 123 - protein, 10 aa, synthetic construct
  REGION 1..10: note = LCDR1
QASQSISYMY

SEQ ID NO: 124 - protein, 10 aa, synthetic construct
  REGION 1..10: note = LCDR1
QASQDISHLY

SEQ ID NO: 125 - protein, 10 aa, synthetic construct
  REGION 1..10: note = LCDR1
QASQDISYLY

SEQ ID NO: 126 - protein, 10 aa, synthetic construct
  REGION 1..10: note = LCDR1
QASQDISYMY

SEQ ID NO: 127 - protein, 10 aa, synthetic construct
  REGION 1..10: note = LCDR1
QASQDVSFLY

SEQ ID NO: 128 - protein, 10 aa, synthetic construct
  REGION 1..10: note = LCDR1
QASQDVSHLN

SEQ ID NO: 129 - protein, 10 aa, synthetic construct
  REGION 1..10: note = LCDR1
QASQDVSHLY

SEQ ID NO: 130 - protein, 10 aa, synthetic construct
  REGION 1..10: note = LCDR1
QASQDVSHMY

SEQ ID NO: 131 - protein, 10 aa, synthetic construct
  REGION 1..10: note = LCDR1
QASQDVSYLY

SEQ ID NO: 132 - protein, 10 aa, synthetic construct
  REGION 1..10: note = LCDR1
QASQDVSYMY

SEQ ID NO: 133 - protein, 10 aa, synthetic construct
  REGION 1..10: note = LCDR1
QASQSISFMY

SEQ ID NO: 134 - protein, 10 aa, synthetic construct
  REGION 1..10: note = LCDR1
QASQSVRHMY

SEQ ID NO: 135 - protein, 10 aa, synthetic construct
  REGION 1..10: note = LCDR1
QASQSVSFMY

SEQ ID NO: 136 - protein, 10 aa, synthetic construct
  REGION 1..10: note = LCDR1
QASQSVSHMY

SEQ ID NO: 137 - protein, 10 aa, synthetic construct
  REGION 1..10: note = LCDR1
QASQSVSYLY

SEQ ID NO: 138 - protein, 10 aa, synthetic construct
  REGION 1..10: note = LCDR1
QASSDISFMY

SEQ ID NO: 139 - protein, 10 aa, synthetic construct
  REGION 1..10: note = LCDR1
QASSDISYMY

SEQ ID NO: 140 - protein, 10 aa, synthetic construct
  REGION 1..10: note = LCDR1
QASSDVSFLY

SEQ ID NO: 141 - protein, 10 aa, synthetic construct
  REGION 1..10: note = LCDR1
QASSDVSFMY

SEQ ID NO: 142 - protein, 10 aa, synthetic construct
  REGION 1..10: note = LCDR1
QASSDVSHLY

SEQ ID NO: 143 - protein, 10 aa, synthetic construct
  REGION 1..10: note = LCDR1
QASSDVSHMY

SEQ ID NO: 144 - protein, 10 aa, synthetic construct
  REGION 1..10: note = LCDR1
QASSDVSYMY

SEQ ID NO: 145 - protein, 10 aa, synthetic construct
  REGION 1..10: note = LCDR1
QASSSISFLY

SEQ ID NO: 146 - protein, 10 aa, synthetic construct
  REGION 1..10: note = LCDR1
QASSSISHLY

SEQ ID NO: 147 - protein, 10 aa, synthetic construct
  REGION 1..10: note = LCDR1
QASSSISYLY

SEQ ID NO: 148 - protein, 10 aa, synthetic construct
  REGION 1..10: note = LCDR1
QASSSVSYMY

SEQ ID NO: 149 - protein, 7 aa, synthetic construct
  REGION 1..7: note = LCDR2
DASNLAT

SEQ ID NO: 150 - protein, 7 aa, synthetic construct
  REGION 1..7: note = LCDR2
DTSNLAT

SEQ ID NO: 151 - protein, 7 aa, synthetic construct
  REGION 1..7: note = LCDR2
DTSNLET

SEQ ID NO: 152 - protein, 7 aa, synthetic construct
  REGION 1..7: note = LCDR2
DASNLET

SEQ ID NO: 153 - protein, 9 aa, synthetic construct
  REGION 1..9: note = LCDR3
QQWDNYPLT

SEQ ID NO: 154 - protein, 9 aa, synthetic construct
  REGION 1..9: note = LCDR3
QQWDTYPLT

SEQ ID NO: 155 - protein, 9 aa, synthetic construct
  REGION 1..9: note = LCDR3
QQWSNYPLT

SEQ ID NO: 156 - protein, 9 aa, synthetic construct
  REGION 1..9: note = LCDR3
QQWDNLPLT

SEQ ID NO: 157 - protein, 9 aa, synthetic construct
  REGION 1..9: note = LCDR3
QQWDTLPLT

SEQ ID NO: 158 - protein, 9 aa, synthetic construct
  REGION 1..9: note = LCDR3
QQWSNLPLT

SEQ ID NO: 159 - protein, 9 aa, synthetic construct
  REGION 1..9: note = LCDR3
QQWSTLPLT

SEQ ID NO: 160 - skipped

SEQ ID NO: 161 - protein, 11 aa, synthetic construct
  REGION 1..11: note = LCDR1
  VARIANT 7..8: note = S or a conservative substitution of S
  VARIANT 9: note = Y or a conservative substitution of Y
  VARIANT 10: note = L or a conservative substitution of L
  VARIANT 11: note = N or T or a conservative substitution of N or T
RASQSIXXXXX

SEQ ID NO: 162 - skipped

SEQ ID NO: 163 - protein, 9 aa, synthetic construct
  REGION 1..9: note = LCDR3
  VARIANT 3: note = S or any other amino acid
  VARIANT 6: note = T or any other amino acid
  VARIANT 8: note = W or any other amino acid
QQXYSXPXT

(19) United States
(12) Patent Application Publication
(10) Pub. No.: US 2023/0357420 A1
(43) Pub. Date: Nov. 9, 2023

(54) ANTI-CD122 ANTIBODIES AND USES THEREOF

(71) Applicant: Villaris Therapeutics, Inc., Cary, NC (US)

(72) Inventors: William James Jonathan FINLAY, Glasgow (GB); John E. HARRIS, Sterling, MA (US)

(21) Appl. No.: 18/336,478

(22) Filed: Jun. 16, 2023

Related U.S. Application Data
(63) Continuation of application No. PCT/US2022/024620, filed on Apr. 13, 2022.
(60) Provisional application No. 63/279,762, filed on Nov. 16, 2021.
(60) Provisional application No. 63/174,772, filed on Apr. 14, 2021.

(51) Int. Cl. A61P 17/00; A61K 47/68; A61K 9/00; C07K 16/28

(52) U.S. Cl. CPC C07K 16/2866; A61K 9/0014; A61K 47/6849; A61P 17/00; C07K 2317/24; C07K 2317/31; C07K 2317/35

(57) ABSTRACT

Provided herein are antibody molecules that bind specifically to CD122 and antigen-binding portions thereof and related compositions, nucleic acid molecules, vectors and host cells. Also provided herein are medical uses of such antibody molecules.

### CROSS REFERENCE TO RELATED APPLICATIONS

This application is a continuation of International Patent Application No. PCT/US2022/024620, filed Apr. 13, 2022, which claims priority to U.S. Provisional Pat. Application No. 63/279,762, filed Nov. 16, 2021, and U.S. Provisional Pat. Application No. 63/174,772, filed Apr. 14, 2021, each of which is incorporated by reference herein in its entirety for all purposes.

### REFERENCE TO AN ELECTRONIC SEQUENCE LISTING

The contents of the electronic sequence listing (VLRS_001_03US_SeqList_ST26.xml; Size: 207,308 bytes; and Date of Creation: Jun. 16, 2023) are herein incorporated by reference in their entirety.

### TECHNICAL FIELD

The present disclosure relates to therapeutic antibody molecules and medical uses thereof.

### BACKGROUND

CD122 is a cell surface receptor that is a member of the immunoglobulin superfamily and is principally expressed on natural killer (NK) and T cells. CD122 has been proposed as a target for a variety of conditions driven by either of these immune cell types, including type 1 diabetes (T1D), celiac disease, leukemia, vitiligo, and others. There is a need for therapeutics that target such immune-mediated diseases. In particular, vitiligo has no systemic treatment options and no U.S. Food and Drug Administration-approved medical treatments that improve disease.

### SUMMARY

Provided herein is an anti-CD122 antibody or an antigen-binding portion thereof, wherein the antibody or antigen-binding portion comprises a heavy chain variable (VH) region and a light chain variable (VL) region wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15.

Provided herein is an anti-CD122 antibody or an antigen-binding portion thereof, wherein the antibody or antigen-binding portion comprises a VH region and a VL region wherein: (a) the VH region amino acid sequence comprises SEQ ID NO: 1 and the VL region amino acid sequence comprises SEQ ID NO: 17; or (b) the VH region amino acid sequence comprises SEQ ID NO: 1 and the VL region amino acid sequence comprises SEQ ID NO: 9.

In some embodiments, the antibody or antigen-binding portion is humanized or chimeric.

In some embodiments, the VH region, the VL region, or both the VH and the VL region comprise one or more human framework region amino acid sequences. In some embodiments, the VH region, the VL region, or both the VH and the VL region comprise a human variable region framework scaffold amino acid sequence into which the CDR amino acid sequences have been inserted. In some embodiments, the VH region comprises an IGHV3-23 human germline scaffold amino acid sequence into which the HCDR1, HCDR2 and HCDR3 amino acid sequences have been inserted. In some embodiments, the VL region comprises an IGKV1-33 human germline scaffold amino acid sequence into which the LCDR1, LCDR2 and LCDR3 amino acid sequences have been inserted.

In some embodiments, the anti-CD122 antibody comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A, L235A and G237A or a wild-type human IgG2 constant region, wherein numbering is according to the EU index as in Kabat. In some embodiments, the immunoglobulin constant region comprises any one of SEQ ID NOs: 32-38.

In some embodiments, the antibody or antigen-binding portion is an Fab, an Fab′, an F(ab′)₂, an Fv, an scFv, a maxibody, a minibody, a diabody, a triabody, a tetrabody, or a bis-scFv. In some embodiments, the antibody is monoclonal. In some embodiments, the antibody is a tetrameric antibody, a tetravalent antibody or a multispecific antibody. In some embodiments, the antibody is a bispecific antibody that binds specifically to a first antigen and a second antigen, wherein the first antigen is CD122 and the second antigen is not CD122.

Provided herein is an immunoconjugate comprising the antibody or antigen-binding portion disclosed herein, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Provided herein is a pharmaceutical composition comprising the antibody, the antigen-binding portion or the immunoconjugate disclosed herein, and a pharmaceutically acceptable carrier, diluent or excipient.

Provided herein is a nucleic acid molecule encoding (a) the VH region amino acid sequence; (b) the VL region amino acid sequence; or (c) both the VH and the VL region amino acid sequences of the antibody or antigen-binding portion disclosed herein. Provided herein is an expression vector comprising the nucleic acid molecule disclosed herein. Provided herein is a recombinant host cell comprising the nucleic acid molecule or the expression vector disclosed herein.

Provided herein is a method of producing an anti-CD122 antibody or an antigen-binding portion thereof, the method comprising: culturing a recombinant host cell comprising the expression vector disclosed herein under conditions whereby the nucleic acid molecule is expressed, thereby producing the antibody or antigen-binding portion; and isolating the antibody or antigen-binding portion from the host cell or culture.

Provided herein is a method for supressing an immune response in a subject, comprising administering to the subject a therapeutically effective amount of the antibody, the antigen-binding portion, the immunoconjugate or the pharmaceutical composition disclosed herein. In some embodiments, the immune response is mediated by CD122.

Provided herein is a method for treating or preventing a disease in a subject, comprising administering to the subject a therapeutically effective amount of the antibody, the antigen-binding portion, the immunoconjugate or the pharmaceutical composition disclosed herein. In some embodiments, the disease is an inflammatory disease or an autoimmune disease. In some embodiments, the disease is vitiligo, celiac disease, type 1 diabetes, multiple sclerosis, graft-versus-host disease, systemic lupus erythematosus, psoriasis, atopic dermatitis, alopecia areata, ulcerative colitis, or rheumatoid arthritis.

Provided herein is a method for supressing IL-15 induced migration of T cells from skin, the method comprising contacting the skin with a therapeutically effective amount of the antibody, the antigen-binding portion, the immunoconjugate or the pharmaceutical composition disclosed herein.

Provided herein is the antibody, the antigen-binding portion, the immunoconjugate or the pharmaceutical composition disclosed herein, for use as a medicament.

### BRIEF DESCRIPTION OF THE DRAWINGS

FIG. 1 depicts a diagram for in vivo pharmacology analysis of anti-CD122 IgG.

FIG. 2A - FIG. 2D depict data from a specificity analysis of Villmab-1 (MIKβ1) anti-CD122 IgG. Proteomic specificity profiling of Villmab 1 using Retrogenix technology. (FIG. 2A) ZS control expression, (FIG. 2B) Villmab-1 probe, (FIG. 2C) Rituximab probe, (FIG. 2D) no primary antibody.

FIG. 3A — FIG. 3C depict data from a flow cytometry analysis of Villmab-1 (MIKβ1) anti-CD122 IgG on target-transfected cells. (FIG. 3A) Villmab-1 binding, (FIG. 3B) no primary antibody (FIG. 3C) Rituximab binding.

FIG. 4 depicts data from an anti-CD122 IgG Alphascreen epitope competition. Novel clones were screened in Alphascreen assay to examine for competition with the Villmab-1 binding epitope on human CD122.

FIG. 5 depicts a bar graph showing polyreactivity scores for novel anti-CD122 IgGs. Antibodies were examined for their ability to non-specifically bind to DNA and Insulin.

FIG. 6A - FIG. 6G depict data from a flow cytometry analysis of novel anti-CD122 IgGs and Villmab-1 on target-transfected cells. (FIG. 6A) 06F11, (FIG. 6B) 07C07, (FIG. 6C) 07D06, (FIG. 6D) 07E09, (FIG. 6E) 07D07, (FIG. 6F) 06D12, (FIG. 6G) Isotype control IgG.

FIG. 7A - FIG. 7F depict data from an analysis of novel anti-CD122 IgGs and Villmab-1 in M07e cell IL-15 proliferation assay. (FIG. 7A) 06F11, (FIG. 7B) 07C07, (FIG. 7C) 07D06, (FIG. 7D) 07E09, (FIG. 7E) 07D07, (FIG. 7F) 06D12.

FIG. 8A - FIG. 8F depict data from an in vivo analysis of novel anti-CD122 IgGs and Villmab-1 in hIL-15 NSG mouse model. (FIG. 8A) human CD8+ T cells prior, (FIG. 8B) human NK cells prior, (FIG. 8C) human CD8+ T cells after 1 week, (FIG. 8D) human NK cells after 1 week, (FIG. 8E) human CD8+ T cells after 3 weeks, (FIG. 8F) human NK cells after 3 weeks.

FIG. 9A - FIG. 9D depict data from a flow cytometry analysis of novel anti-CD122 IgGs and Villmab-1 on target-transfected cells. (FIG. 9A) 06F11, (FIG. 9B) 07C07, (FIG. 9C) Isotype control IgG, (FIG. 9D) Rituximab.

FIG. 10A - FIG. 10C depict data from a specificity analysis of Villmab-1 (MIKβ1) anti-CD122 novel clones in Fab and IgG formats. Specificity profiling of Fabs using BIACORE^{®} technology against human neudesin protein (FIG. 10A) or CILP2 protein (FIG. 10B) was measured as Rmax (maximum specific binding response values). Specificity profiling of IgGs using ELISA against human BCAM protein (FIG. 10C), measured as OD450 nm.

FIG. 11A - FIG. 11B depict data from a sequence analysis of Villmab-1 (MIKβ1) anti-CD122 novel clone variable domain sequences. VH sequences (FIG. 11A) and VL sequences (FIG. 11B). In FIG. 11A, the sequences are as follows: VillMAB-1: SEQ ID NO: 22; MAB05: SEQ ID NO: 1; MAB06: SEQ ID NO: 1; MAB14: SEQ ID NO: 52; MAB15: SEQ ID NO: 52; MAB 17: SEQ ID NO: 53; MAB 18: SEQ ID NO: 53. In FIG. 11B, the sequences are as follows: VillMAB-1: SEQ ID NO: 28; MAB05: SEQ ID NO: 9; MAB06: SEQ ID NO: 17; MAB14: SEQ ID NO: 9; MAB15: SEQ ID NO: 17; MAB17: SEQ ID NO: 9; MAB18: SEQ ID NO: 17. CDRs are bold and underlined. Residues differing from VillMab-1 sequence are highlighted in grey boxes. Unique residues found only in clones in this analysis that do not bind BCAM, CILP2 or neudesin are boxed in black.

FIG. 12A - FIG. 12B depict data from an analysis of novel anti-CD122 IgGs and Villmab-1 in primary NK cell IL-15 proliferation assay. MAB05 (FIG. 12A), MAB06 (FIG. 12B).

FIG. 13A - FIG. 13B depict data from an analysis of MAB05 and MAB06 effect on IL15-induced accumulation of T cells migrating from skin biopsies in a human skin biopsy culture assay.

FIG. 13A shows the effect on CD8+ T cell number. FIG. 13B shows the effect on CD4+ T cell number.

FIG. 14A - FIG. 14B depict data from an analysis of MAB05 and MAB06 concentration-dependent antagonism of IL 15-induced accumulation of T cells migrating from skin biopsies in a human skin biopsy culture assay. FIG. 13A shows the effect on CD8+ T cells and the relevant IC50s. FIG. 13B shows the effect on CD4+ T cells and the relevant IC50s.

### DETAILED DESCRIPTION

Provided herein are anti-CD122 antibodies and therapeutic uses of such antibodies. The antibodies disclosed herein are antagonistic, well expressed, biophysically stable, highly soluble and of maximized identity to preferred human germlines.

CD122 (also known as IL2RB, IL-2Rβ, IL15RB, P70-75, interleukin 2 receptor subunit beta, and IMD63) is a type I transmembrane glycoprotein and member of the Ig superfamily. CD122 is a shared subunit of the interleukin-15 (IL-15) receptor and the interleukin-2 (IL-2) receptor. CD122 is expressed by NK cells and a subset of T cells. IL-15 signaling has been implicated in human vitiligo pathogenesis. Targeting CD122 or blocking IL-15 signaling appeared to be beneficial in mouse models of other immune-mediated diseases, such as diabetes, psoriasis, multiple sclerosis and alopecia areata, as well as improving symptoms of rheumatoid arthritis and celiac disease. Developing an effective antagonistic anti-CD122 antibody would be valuable in treating immune-mediated diseases.

U.S. Pat. No. 5,585,089, herein incorporated by reference in its entirety, describes an antagonistic murine anti-CD122 IgG molecule termed “MIKβ1”, as well as preparation of humanized forms of MIKβ1. Those humanized forms of MIKβ1 were produced using classical humanization techniques, i.e., by grafting of Kabat-defined murine CDRs into human heavy and light chain framework sequences, with some of the human framework residues being potentially back-mutated to the correspondingly positioned MIKβ1 murine residues. A partially humanized version of MIKβ1 (see Table 20) did not show efficacy in Phase IIa clinical trials for T-cell large granular lymphocytic (T-LGL) leukemia and Human T cell lymphotropic virus 1 (HTLV-1)-associated myelopathy/tropical spastic paraparesis (HAM/TSP). This antibody has a number of liabilities, including the fact that it is partially humanized, has off-target binding that may affect pharmacokinetics and biodistribution, and uses the IgG1 isotype, leading to a risk of unwanted antibody-dependent cellular cytotoxicity/antibody-dependent cellular phagocytosis on cells which do not mediate disease. These features make this antibody a suboptimal candidate for further testing as a targeted therapeutic in human immune-mediated diseases.

In contrast, the anti-CD122 antibodies provided herein demonstrate advantages as described herein that make them useful for therapies of human immune-mediated diseases and disorders.

### Antibodies

Provided herein are antibodies and antigen-binding portions thereof that specifically bind CD122. The anti-CD122 antibodies provided herein have several advantages over the murine anti-CD122 antibody MIKβ1 and a humanized version thereof disclosed in US 5,585,089. The anti-CD122 antibodies provided herein have been selected to have improved potency in blocking IL-15 signalling through CD122. Critically, these antibodies also dramatically improved the specificity of CD122 binding in comparison to MIKβ1, by ablating off-target binding to the human receptor BCAM (also known as AU, CD239, LU, MSK19, basal cell adhesion molecule (Lutheran blood group)), neudesin (also known as NENF) and CILP2 (also known as cartilage intermediate layer protein 2).

Antibodies and antigen-binding portions disclosed herein specifically bind human CD122. In some embodiments, antibodies and antigen-binding portions may cross-react with CD122 from species other than human, for example, cynomolgus monkey (Macaca fascicularis) CD122 and/or rhesus monkey (Macaca mulatta) CD122. In some embodiments, an antibody may be specific for only human CD122 and may exhibit no non-human cross-reactivity. Exemplary amino acid sequences of human, cynomolgus and rhesus CD122 are provided in Table 16.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable region (abbreviated herein as VH region) and a heavy chain constant region. The heavy chain constant region comprises three domains, CH1, CH2 and CH3. Each light chain comprises a light chain variable region (abbreviated herein as VL region) and a light chain constant region. The light chain constant region comprises one domain, CL. The VH and VL regions can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4. The CDR definition used in the present application is the Kabat definition (Kabat et al., Sequences of Proteins of Immunological Interest, 5^{th} ed. Bethesda, MD: Public Health Service, National Institutes of Health (1991)).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., CD122). It has been shown that the antigen-binding function of an antibody can be performed by portions or fragments of a full-length antibody. Examples of binding portions encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′)₂ fragments. Fab′ is derived from F(ab′)₂; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3(1):1-9. Examples of antigen-binding portions are known in the art (Kontermann and Dubel eds., Antibody Engineering (2001) Springer-Verlag. New York. 790 pp.). In some embodiments, provided herein is a single domain antibody. In general, the term “antibody” when used herein encompasses an “antibody portion”. An antibody portion generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or an antigen-binding portion is a bispecific molecule that binds specifically to a first antigen and a second antigen, wherein the first antigen is CD122 and the second antigen is not CD122. In some embodiments, an antibody or an antigen-binding portion is a diabody. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an anti-CD122 antigen-binding portion disclosed herein is an Fab, an Fab′, an F(ab′)₂, an Fv, an scFv, a maxibody, a minibody, a diabody, a triabody, a tetrabody, or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off} /Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind CD122 when the equilibrium binding constant (K_{d}) is ≤10 µM, preferably ≤ 10 nM, more preferably ≤ 10 nM, and most preferably ≤ 100 pM to about 1 pM, as measured by assays such as radioligand binding assays or similar assays known to those skilled in the art. One method for determining the K_{d} of an antibody is by using surface plasmon resonance (SPR), typically using a biosensor system such as a Biacore® system.

In some embodiments, an anti-CD122 antibody or antigen-binding portion provided herein is monovalent or bivalent and comprises a single or double chain. Functionally, the binding affinity of an antibody or antigen-binding portion may be within the range of 10⁻⁵M to 10⁻¹² M. For example, the binding affinity of an antibody or antigen-binding portion is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

Provided herein is an anti-CD122 antibody or an antigen-binding portion thereof, wherein the antibody or antigen-binding portion cross-competes for binding to CD122 with antibody MAB05 or MAB06, or an antibody that comprises one or more amino acid sequences of antibody MAB05 or MAB06 (see Tables 18 and 19).

The terms “cross-compete”, “cross-competition”, “cross-block”, “cross-blocked” and “cross-blocking” are used interchangeably herein to mean the ability of an antibody or an antigen-binding portion thereof to interfere with the binding directly or indirectly through allosteric modulation of the anti-CD122 antibodies of the disclosure to the target CD122 (e.g., human CD122). The extent to which an antibody or portion thereof is able to interfere with the binding of another to the target, and therefore whether it can be said to cross-block or cross-compete, can be determined using competition binding assays. One example of a binding competition assay is Homogeneous Time Resolved Fluorescence (HTRF). One particularly suitable quantitative cross-competition assay uses a FACS- or an Alphascreen-based approach to measure competition between the labelled (e.g., His-tagged, biotinylated or radioactive labelled) antibody or portion thereof and the other antibody or portion thereof in terms of their binding to the target. In general, a cross-competing antibody or portion thereof is, for example, one which will bind to the target in the cross-competition assay such that, during the assay and in the presence of a second antibody or portion thereof, the recorded displacement of the immunoglobulin single variable domain or polypeptide according to the invention is up to 100% (e.g. in a FACS-based competition assay) of the maximum theoretical displacement (e.g. displacement by cold (e.g., unlabeled) antibody or fragment thereof that needs to be cross-blocked) by the potentially cross-blocking antibody or fragment thereof that is present in a given amount. In some embodiments, cross-competing antibodies or portions thereof have a recorded displacement that is between 10% and 100%, or between 50% and 100%.

Provided herein is an anti-CD122 antibody or an antigen-binding portion thereof, wherein the antibody or antigen-binding portion cross-competes for binding to CD122 with an antibody comprising a VH region and a VL region, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15.

Provided herein is an anti-CD122 antibody or an antigen-binding portion thereof, wherein the antibody or antigen-binding portion cross-competes for binding to CD122 with the antibody or antigen-binding portion comprising the sets of CDRs disclosed herein; and (a) binds specifically to (i) human CD122 and (ii) cynomolgus and/or rhesus CD122; (b) antagonizes proliferation of human CD122⁺ cells, such as primary NK cells, when stimulated by human IL-15, with an EC50 lower than 14 nM; (c) binds to rhesus CD122 with a KD lower than 10 nM; (d) binds to a functionally identical epitope on cynomolgus and/or rhesus CD122 and human CD122; and/or (e) exhibits no or reduced binding to BCAM compared to an anti-CD122 antibody comprising the variable domain sequences of antibody MIKβ1. In some embodiments, a K_{d} value of an antibody or antigen-binding portion may be determined by BIACORE^{®} analysis. In some embodiments, an EC50 value of an antibody or antigen-binding portion may be determined by flow cytometric staining of CD122-expressing cells (e.g., CHO cells, HEK cells, M07e cells, NK cells, T cells).

In some embodiments, an anti-CD122 antibody or antigen-binding portion provided herein has low immunogenicity. In some embodiments, an antibody or antigen-binding portion exhibits reduced immunogenicity compared to an anti-CD122 antibody comprising HCDR1 of SYGVH (SEQ ID NO: 24), HCDR2 of VIWSGGSTDYNAAFIS (SEQ ID NO: 5), HCDR3 of AGDYNYDGFAY (SEQ ID NO: 27), LCDR1 of SGSSSVSFMY (SEQ ID NO: 30), LCDR2 of DTSNLAS (SEQ ID NO: 13), and LCDR3 of QQWSTYPLT (SEQ ID NO: 15). In some examples, immunogenicity risk of an antibody or antigen-binding portion may be determined in silico by identifying the location of T cell epitopes in the antibody or portion (e.g., in the variable regions of the antibody or portion).

For example, T cell epitopes in an antibody or antigen-binding portion may be identified by using iTope™. iTope™ can used to analyze VL and VH region sequences for peptides with promiscuous high affinity binding to human MHC class II. Promiscuous high affinity MHC class II binding peptides are thought to correlate with the presence of T cell epitopes that are high risk indicators for clinical immunogenicity of drug proteins. The iTope™ software predicts favorable interactions between amino acid side chains of a peptide and specific binding pockets (in particular pocket positions; p1, p4, p6, p7 and p9) within the open-ended binding grooves of 34 human MHC class II alleles. These alleles represent the most common HLA-DR alleles found world-wide with no weighting attributed to those found most prevalently in any particular ethnic population. Twenty of the alleles contain the “open” p1 configuration and 14 contain the “closed” configuration where glycine at position 83 is replaced by a valine. The location of key binding residues is achieved by the in silico generation of 9mer peptides that overlap by eight amino acids spanning the test protein sequence. This process successfully discriminates with high accuracy between peptides that either bind or do not bind MHC class II molecules.

T cell epitopes in an antibody or antigen-binding portion may be identified by analysing VL and VH region sequences using TCED™ (T Cell Epitope Database™) to search for matches to T cell epitopes previously identified by in vitro human T cell epitope mapping analyses of other protein sequences. The TCED™ is used to search any test sequence against a large (>10,000 peptides) database of peptides derived from unrelated protein and antibody sequences.

In some embodiments, an anti-CD122 antibody or antigen-binding portion may exhibit a low immunogenicity because the antibody or portion has a low number of one or more of the following peptides in its sequences: High Affinity Foreign (‘HAF’ - high immunogenicity risk), Low Affinity Foreign (‘LAF’ - lower immunogenicity risk), and/or TCED+ (previously identified epitope in TCED™ database).

In some embodiments, an anti-CD122 antibody or antigen-binding portion may have high Germline Epitope (GE) content in its sequence. In some examples, an anti-CD122 antibody or antigen-binding portion has 3, 4, 5, 6, 7, 8, 9, 10, 11, 12, 13, 14, 15, 16, 17, 18, 19 or 20 (or greater than 20) germline epitopes in its sequence (e.g., in the VL and/or VH region sequence). Germline Epitope may be defined as a human germline peptide sequence with high MHC Class II binding affinity. Germline Epitope 9mer peptides are unlikely to have immunogenic potential due to T cell tolerance, as validated by previous studies with a wide range of germline peptides. Importantly, such germline v-domain epitopes (aided further by similar sequences in the human antibody constant regions) also compete for MHC Class II occupancy at the membrane of antigen presenting cells, reducing the risk of foreign peptide presentation being sufficient to achieve the ‘activation threshold’ required for T cell stimulation. High GE content is therefore a beneficial quality in clinical development of an antibody therapeutic and can provide low immunogenicity. In some examples, an anti-CD122 antibody or antigen-binding portion comprises a human germline peptide sequence with high MHC class II binding affinity (e.g., germline epitope) in the LCDR2.

In certain embodiments, an anti-CD122 antibody or antigen-binding portion may have a reduced number of HAF, LAF and/or TCED+ epitopes found in the frameworks of both the heavy and light chain variable regions compared to an anti-CD122 antibody comprising the variable domain sequences of antibody MIKβ1. In some embodiments, HAF, LAF and/or TCED+ epitopes are not present in the VL and/or VH region sequences of an anti-CD122 antibody or antigen-binding portion.

In some embodiments, an anti-CD122 antibody or an antigen-binding portion thereof does not comprise one or more of the MIKβ1 murine/humanized antibody amino acid sequences provided in Table 20. In some embodiments, an anti-CD122 antibody or an antigen-binding portion thereof does not comprise a HCDR1 comprising SEQ ID NO: 24, a HCDR3 comprising SEQ ID NO: 27 and/or a LCDR1 comprising SEQ ID NO: 30. Table 1 provides the amino acid sequences of the MIKβ1 murine anti-CD122 antibody variable regions with highlighted CDRs as defined herein (“Kabat” scheme). The term “MIKβ1-IgG1 (humanized)” refers to an anti-CD122 antibody comprising the variable heavy region sequence labelled CD122-VH1 and the variable light region sequence labelled CD122-VL1 in Table 2 and a human IgG1 constant region.

The antibodies disclosed herein are anti-CD122 antagonist antibodies. As used herein, an “antagonist” or an “anti-CD122 antagonist antibody” (interchangeably termed “anti-CD122 antibody”) refers to an antibody which is able to bind to CD122 and inhibit CD122 biological activity and/or downstream pathway(s) mediated by CD122 signalling. An anti-CD122 antagonist antibody encompasses antibodies that can block, antagonize, suppress or reduce (including significantly) CD122 biological activity, including downstream pathways mediated by CD122 signalling, such as receptor binding and/or elicitation of a cellular response to CD122. For the purposes of the present disclosure, it will be explicitly understood that the term “anti-CD122 antagonist antibody” encompass all the terms, titles, and functional states and characteristics whereby CD122 itself, and CD122 biological activity (including but not limited to its ability to suppress the activation of anti-tumor cell activity of T cells), or the consequences of the activity or biological activity, are substantially nullified, decreased, or neutralized in a meaningful degree.

In some embodiments, an antibody molecule or antigen-binding portion thereof binds specifically to CD122 and does not bind (or does not bind specifically) to the membrane protein BCAM. In some embodiments, BCAM is a human protein. In some embodiments, BCAM is a rhesus protein. In some embodiments, the human BCAM protein comprises or consists of the amino acid sequence of SEQ ID NO: 21 or an amino acid sequence that is at least about 90%, at least about 91%, at least about 92%, at least about 93%, at least about 94%, at least about 95%, at least about 96%, at least about 97%, at least about 98% or at least about 99% identical to SEQ ID NO: 21. In one embodiment, an antibody molecule or antigen-binding portion thereof does not bind to BCAM. In some embodiments, an antibody molecule or antigen-binding portion thereof exhibits reduced binding to BCAM compared to the binding exhibited by antibody MIKβ1 or IgG1-MIKβ1 (humanized) to said membrane receptors. In some cases, binding of an antibody or antigen-binding portion thereof to BCAM may be determined by ELISA or by flow cytometry analyses.

Further provided herein is an anti-CD122 antibody or an antigen-binding portion thereof that comprises one or more amino acid sequences of antibody MAB06 or MAB05. The combinations of VH region, VL region and CDR sequences forming these antibodies are provided in Tables 18 and 19. In some embodiments, the VH region sequence and/or the VL region sequence comprises a signal sequence (also known as a signal peptide) at the amino-terminus.

Provided herein is an anti-CD122 antibody or an antigen-binding portion thereof, wherein the antibody or antigen-binding portion comprises a heavy chain variable (VH) region and a light chain variable (VL) region wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15.

In some embodiments, disclosed herein is an anti-CD122 antibody or an antigen-binding portion thereof, wherein the antibody comprises a VH region comprising HCDR1, HCDR2, and HCDR3 and a VL region comprising LCDR1, LCDR2, and LCDR3, wherein (a) the HCDR1 comprises the amino acid sequence G-F-T-F-S-S-Y-X₁-M-S, wherein X₁ is L or any other amino acid (SEQ ID NO: 39); (b) the HCDR2 comprises the amino acid sequence X₁-A-X₂-I-S-G-G-G-X₃-X₄-X₅-Y-Y-X₆-D-S-V-K-G, wherein X₁ is V or a conservative substitution of V, X₂ is T or N, X₃ is A or S, X₄ is E or N, X₅ is T or K, and X₆ is P or V (SEQ ID NO: 40); (c) the HCDR3 comprises the amino acid sequence X₁-X₂-X₃-X₄-X₅-D-Y, wherein X₁ is Q or any other amino acid (for example, T, L, M, or N), X₂ is L or any other amino acid (for example, G, K, M, Q, S, or V), X₃ is Y or a conservative substitution of Y (for example, H), X₄ is Y or any other amino acid (for example, A, D, F, G, M, E, I, K, S, or W) and X₅ is F or any other amino acid (for example A, D, E, I, K, M, S, or W) (SEQ ID NO: 160); (d) the LCDR1 comprises the amino acid sequence R-A-S-Q-S-I-X₁-X₂-X₃-X₄-X₅, wherein X₁ is S or a conservative substitution of S, X₂ is S or a conservative substitution of S, X₃ is Y or a conservative substitution of Y, X₄ is L or a conservative substitution of L, and X₅ is N or T or a conservative substitution of N or T (SEQ ID NO: 161); (e) the LCDR2 comprises the amino acid sequence X₁-A-X₂-S-L-X₃-X₄, wherein X₁ is A or T or a conservative substitution of A or T, X₂ is S or a conservative substitution of S, X₃ is Q or any other amino acid, and X₄ is S or any other amino acid (SEQ ID NO: 162); and (f) the LCDR3 comprises the amino acid sequence Q-Q-X₁-Y-S-X₂-P-X₃-T, wherein X₁ is S or any other amino acid, X₂ is T or any other amino acid, and X₃ is W or any other amino acid (SEQ ID NO: 163).

Provided herein is an anti-CD122 antibody or an antigen-binding portion thereof, wherein the antibody or antigen-binding portion comprises a VH region and a VL region, wherein (a) the VH region amino acid sequence comprises or consists of SEQ ID NO: 1 and the VL region amino acid sequence comprises or consists of SEQ ID NO: 17; or (b) the VH region amino acid sequence comprises or consists of SEQ ID NO: 1 and the VL region amino acid sequence comprises or consists of SEQ ID NO: 9.

Provided herein is an anti-CD122 antibody or an antigen-binding portion thereof, wherein the antibody or antigen-binding portion comprises a VH region and a VL region, wherein the VH region amino acid sequence comprises SEQ ID NO: 1, or an amino acid sequence that is at least 95%, 96%, 97%, 98% or 99% identical to the amino acid sequence of SEQ ID NO: 1.

Provided herein is an anti-CD122 antibody or an antigen-binding portion thereof, wherein the antibody or antigen-binding portion comprises a VH region and a VL region, wherein the VL region amino acid sequence comprises (a) SEQ ID NO: 17, or an amino acid sequence that is at least 95%, 96%, 97%, 98% or 99% identical to the amino acid sequence of SEQ ID NO: 9; or (b) SEQ ID NO: 17, or an amino acid sequence that is at least 95%, 96%, 97%, 98% or 99% identical to the amino acid sequence of SEQ ID NO: 9.

Also provided herein is an anti-CD122 antibody or an antigen-binding portion thereof, wherein the antibody or antigen-binding portion comprises a VH region and a VL region, wherein (a) the VH region amino acid sequence comprises SEQ ID NO: 1, or an amino acid sequence that is at least 95%, 96%, 97%, 98% or 99% identical to the amino acid sequence of SEQ ID NO: 1; and the VL region amino acid sequence comprises SEQ ID NO: 17, or an amino acid sequence that is at least 95%, 96%, 97%, 98% or 99% identical to the amino acid sequence of SEQ ID NO: 9; or (b) the VH region amino acid sequence comprises SEQ ID NO: 1, or an amino acid sequence that is at least 95%, 96%, 97%, 98% or 99% identical to the amino acid sequence of SEQ ID NO: 1; and the VL region amino acid sequence comprises SEQ ID NO: 9, or an amino acid sequence that is at least 95%, 96%, 97%, 98% or 99% identical to the amino acid sequence of SEQ ID NO: 17.

Provided herein is an anti-CD122 antibody or an antigen-binding portion thereof, wherein the antibody or antigen-binding portion comprises a VH region and a VL region, wherein (a) the VH region amino acid sequence comprises SEQ ID NO: 1; and the VL region amino acid sequence comprises SEQ ID NO: 17, with 1, 2 or 3 conservative amino acid substitutions in the VH region sequence, the VL region sequence, or both the VH region and the VL region sequences; or (b) the VH region amino acid sequence comprises SEQ ID NO: 1; and the VL region amino acid sequence comprises SEQ ID NO: 9, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

In some embodiments, an anti-CD122 antibody or antigen-binding portion provided herein is monoclonal. The term “monoclonal antibody” (Mab) refers to an antibody, or antigen-binding portion thereof, that is derived from a single copy or clone, including for example any eukaryotic, prokaryotic, or phage clone, and not the method by which it is produced. Preferably, a monoclonal antibody exists in a homogeneous or substantially homogeneous population.

In some embodiments, the antibody or antigen-binding portion provided herein may be isolated.

In some embodiments, an anti-CD122 antibody or antigen-binding portion provided herein is chimeric. The term “chimeric” is intended to refer to an antibody molecule, or an antigen-binding portion thereof, in which the variable domain sequences are derived from one species and at least one constant region sequence is derived from another species. For example, one or all the variable domains of the light chain(s) and/or one or all the variable domains of the heavy chain(s) of a mouse antibody (e.g., a mouse monoclonal antibody) may each be joined to a human constant region, such as, without limitation an IgG1 or an IgG4 human constant region. Examples of chimeric antibodies and suitable techniques for their generation are provided in U.S. 4,816,567; U.S. 4,975,369; and U.S. 4,816,397, each of which is incorporated herein by reference in its entirety. In some embodiments, an anti-CD122 antibody or an antigen-binding portion provided herein comprises: (a) a VH region amino acid sequence comprising SEQ ID NO: 1; a VL region amino acid sequence comprising SEQ ID NO: 17 and a human constant region; or (b) a VH region amino acid sequence comprising SEQ ID NO: 1, a VL region amino acid sequence comprising SEQ ID NO: 9 and a human constant region.

In some embodiments, an anti-CD122 antibody or antigen-binding portion provided herein is humanized. The term “humanized” is intended to refer to an antibody, or an antigen-binding portion thereof, that has been engineered to comprise one or more human framework regions in the variable domain together with non-human (e.g., mouse, rat, or hamster) CDRs of the heavy and/or light chain. In some embodiments, a humanized antibody comprises sequences that are entirely human except for the CDRs. An anti-CD122 antibody molecule or antigen-binding portion thereof may comprise one or more human variable region framework scaffolds into which the CDRs have been inserted. In some embodiments, the VH region, the VL region, or both the VH region and the VL region of an anti-CD122 antibody or antigen-binding portion provided herein comprise one or more human framework region amino acid sequences. In some embodiments, a humanized antibody comprises sequences that are entirely human except for the CDRs, which are the CDRs of antibody MAB06 or MAB05. Examples of humanized antibodies and suitable techniques for their generation are provided in Hwang et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. 5,225,539; U.S. 5,530,101; U.S. 5,585,089; U.S. 5,693,761; U.S. 5,693,762; U.S. 6,180,370; and WO 90/07861, each of which is incorporated herein by reference in its entirety. When choosing FR to flank CDRs, for example when humanizing or optimizing an antibody, FRs from antibodies that contain CDR sequences in the same canonical class are preferred.

In some embodiments, an anti-CD122 antibody or an antigen-binding fragment provided herein does not necessarily have the maximum number of human germline substitutions at corresponding murine CDR or other (such as framework) amino acid positions. As elaborated in the experimental section below, “maximally humanized” antibody molecules are not necessary “maximally optimized” in terms of anti-CD122 binding characteristics and/or other desirable features.

The present disclosure encompasses modifications to the amino acid sequence of the antibody molecule or antigen-binding portion thereof as defined herein. For example, the disclosure includes antibody molecules and corresponding antigen-binding portions thereof comprising functionally equivalent variable regions and CDRs which do not significantly affect their properties as well as variants which have enhanced or decreased activity and/or affinity. For example, the amino acid sequence may be mutated to obtain an antibody with the desired binding affinity to CD122. Insertions which include amino- and/or carboxyl-terminal fusions ranging in length from one residue to polypeptides containing a hundred or more residues, as well as intrasequence insertions of single or multiple amino acid residues, are envisaged. Examples of terminal insertions include an antibody molecule with an N-terminal methionyl residue or the antibody molecule fused to an epitope tag. Other insertional variants of the antibody molecule include the fusion to the N- or C-terminus of the antibody of an enzyme or a polypeptide which increases the half-life of the antibody in the blood circulation.

In some embodiments, the anti-CD122 antibody or antigen-binding portion provided herein may include glycosylated and non-glycosylated polypeptides, as well as polypeptides with other post-translational modifications, such as, for example, glycosylation with different sugars, acetylation, and phosphorylation. The antibody or antigen-binding portion may be mutated to alter such post-translational modifications, for example by adding, removing or replacing one or more amino acid residues to form or remove a glycosylation site.

In some embodiments, the anti-CD122 antibody or antigen-binding portion provided herein may be modified for example by amino acid substitution to remove potential proteolytic sites in the antibody or portion.

Also provided herein is an anti-CD122 antibody or an antigen-binding portion thereof, wherein the antibody or antigen-binding portion comprises a VH region, a VL region and all human framework region sequences, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15.

Also provided herein is an anti-CD122 antibody or an antigen-binding portion thereof, wherein the antibody or antigen-binding portion comprises a VH region, a VL region and one or more human framework region sequences, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15.

In some embodiments, an anti-CD122 antibody or an antigen-binding portion thereof may comprise an IGHV3-23 human germline scaffold into which the corresponding HCDR sequences have been inserted. An anti-CD122 antibody or an antigen-binding portion thereof may comprise a VH region that comprises an IGHV3-23 human germline scaffold amino acid sequence into which a set of corresponding HCDR1, HCDR2 and HCDR3 amino acid sequences have been inserted.

In some embodiments, an anti-CD122 antibody or an antigen-binding portion thereof may comprise an IGKV1-33 human germline scaffold into which the corresponding LCDR sequences have been inserted. An anti-CD122 antibody or an antigen-binding portion thereof may comprise a VL region that comprises an IGKV1-33 human germline scaffold amino acid sequence into which a set of corresponding LCDR1, LCDR2 and LCDR3 amino acid sequences have been inserted.

In some embodiments, an anti-CD122 antibody or an antigen-binding portion thereof may comprise an IGHV3-23 human germline scaffold into which the corresponding HCDR sequences have been inserted and an IGKV1-33 human germline scaffold into which the corresponding LCDR sequences have been inserted. An anti-CD122 antibody or an antigen-binding portion thereof may comprise a VH region that comprises an IGHV3-23 human germline scaffold amino acid sequence into which a set of corresponding HCDR1, HCDR2 and HCDR3 amino acid sequences have been inserted and a VL region that comprises an IGKV1-33 human germline scaffold amino acid sequence into which a set of corresponding LCDR1, LCDR2 and LCDR3 amino acid sequences have been inserted. The HCDR1, HCDR2, HCDR3, LCDR1, LCDR2 and LCDR3 amino acid sequences may be the HCDR1, HCDR2, HCDR3, LCDR1, LCDR2 and LCDR3 amino acid sequences of any one of the clones in Table 18 or 19 (with all six CDR sequences being from the same clone).

In some embodiments, an anti-CD122 antibody or an antigen-binding portion thereof comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region comprises one or more mutations to reduce or prevent FcγR binding, antibody-dependent cell-mediated cytotoxicity activity, and/or complement-dependent cytotoxicity activity. In some embodiments, the immunoglobulin constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU index as in Kabat. In some embodiments, a position of an amino acid residue in a constant region of an immunoglobulin molecule is numbered according to the EU index as in Kabat(Ward et al., 1995 Therap. Immunol. 2:77-94).

In some embodiments, an anti-CD122 antibody or an antigen-binding portion thereof may comprise an immunoglobulin light chain constant region that is a kappa light chain constant region or a lambda light chain constant region.

In some embodiments, an anti-CD122 antibody may comprise an immunoglobulin constant region comprising any one of the amino acid sequences in Table 15. The Fc region sequences in Table 15 begin at the CH1 domain. In some embodiments, an anti-CD122 antibody may comprise an immunoglobulin constant region comprising an amino acid sequence of an Fc region of human IgG4, human IgG4(S228P), human IgG2, human IgG1, human IgG1 effector null. For example, the human IgG4(S228P) Fc region comprises the following substitution compared to the wild-type human IgG4 Fc region: S228P. For example, the human IgG1 effector null Fc region comprises the following substitutions compared to the wild-type human IgG1 Fc region: L234A, L235A and G237A. In some embodiments, an immunoglobulin constant region may comprise an RDELT (SEQ ID NO: 39) motif or an REEM (SEQ ID NO: 40) motif (underlined in Table 15). The REEM (SEQ ID NO: 40) allotype is found in a smaller human population than the RDELT (SEQ ID NO: 39) allotype. In some embodiments, an anti-CD122 antibody may comprise an immunoglobulin constant region comprising any one of SEQ ID NOS: 32-38. In some embodiments, an anti-CD122 antibody may comprise the six CDR amino acid sequences of any one of the clones in Table 18 or 19 and any one of the Fc region amino acid sequences in Table 15. In some embodiments, an anti-CD122 antibody may comprise an immunoglobulin heavy chain constant region comprising any one of the Fc region amino acid sequences in Table 15 and an immunoglobulin light chain constant region that is a kappa light chain constant region or a lambda light chain constant region.

In some embodiments, provided herein is an anti-CD122 antibody or an antigen-binding portion thereof, wherein the antibody comprises a VH region, a VL region and a heavy chain constant region, wherein (a) the VH region amino acid sequence comprises a HCDR1 comprising SEQ ID NO: 3, a HCDR2 comprising SEQ ID NO: 5 and a HCDR3 comprising SEQ ID NO: 7; the VL region amino acid sequence comprises a LCDR1 comprising SEQ ID NO: 18, a LCDR2 comprising SEQ ID NO: 13 and a LCDR3 comprising SEQ ID NO: 15; and the heavy chain constant region comprises any one of SEQ ID NOS: 32-38; or (b) the VH region amino acid sequence comprises a HCDR1 comprising SEQ ID NO: 3, a HCDR2 comprising SEQ ID NO: 5 and a HCDR3 comprising SEQ ID NO: 7; the VL region amino acid sequence comprises a LCDR1 comprising SEQ ID NO: 11, a LCDR2 comprising SEQ ID NO: 13 and a LCDR3 comprising SEQ ID NO: 15; and the heavy chain constant region comprises any one of SEQ ID NOS: 32-38.

In some embodiments, provided herein is an anti-CD122 antibody or an antigen-binding portion thereof, wherein the antibody comprises a VH region, a VL region and a heavy chain constant region, wherein (a) the VH region amino acid sequence comprises a HCDR1 comprising SEQ ID NO: 3, a HCDR2 comprising SEQ ID NO: 5 and a HCDR3 comprising SEQ ID NO: 7; the VL region amino acid sequence comprises a LCDR1 comprising SEQ ID NO: 18, a LCDR2 comprising SEQ ID NO: 13 and a LCDR3 comprising SEQ ID NO: 15; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A; or (b) the VH region amino acid sequence comprises a HCDR1 comprising SEQ ID NO: 3, a HCDR2 comprising SEQ ID NO: 5 and a HCDR3 comprising SEQ ID NO: 7; the VL region amino acid sequence comprises a LCDR1 comprising SEQ ID NO: 11, a LCDR2 comprising SEQ ID NO: 13 and a LCDR3 comprising SEQ ID NO: 15; and the heavy chain constant region comprises any one of SEQ ID NOS: 32-38; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A.

In some embodiments, provided herein is an anti-CD122 antibody or an antigen-binding portion thereof, wherein the antibody or antigen-binding portion comprises a VH region and a VL region, wherein (a) the VH region amino acid sequence comprises or consists of SEQ ID NO: 1; the VL region amino acid sequence comprises or consists of SEQ ID NO: 17; and the heavy chain constant region comprises any one of SEQ ID NOS: 32-38; or (b) the VH region amino acid sequence comprises or consists of SEQ ID NO: 1; the VL region amino acid sequence comprises or consists of SEQ ID NO: 9; and the heavy chain constant region comprises any one of SEQ ID NOS: 32-38.

In some embodiments, an anti-CD122 antibody may be immune effector null. In some embodiments, an anti-CD122 antibody or an antigen-binding portion thereof does not induce immune effector function and, optionally, suppresses immune effector function. In some embodiments, an anti-CD122 antibody may lack measurable binding to human FcγRI, FcγRIIa, FcγRIIIa and FcγRIIIb receptors but maintain binding to human FcγRIIb receptor and optionally maintain binding to human FcRn receptor. FcγRI, FcγRIIa, FcγRIIIa and FcγRIIIb are examples of activating receptors. FcγRIIb is an example of an inhibitory receptor. FcRn is an example of a recycling receptor. In some embodiments, binding affinity of an anti-CD122 antibody or an antigen-binding portion thereof for human Fc receptors may be measured by BIACORE® analysis. In some embodiments, Homogeneous Time Resolved Fluorescence (HTRF) can be used to study binding of an anti-CD122 antibody to human Fc receptors. In one example of HTRF, human IgG1 (wild type) is labelled, as is the full suite of Fc gamma receptors and then antibodies with engineered Fc fragments are used in titration competition. In some embodiments, CD122-positive cells may be mixed with human white blood cells and anti-CD122 antibodies, and cell killing by CDC, ADCC and/or ADCP may be measured. In some embodiments, an anti-CD122 antibody comprising an amino acid sequence of an Fc region of human IgG1 (see Table 15) is effector null. In some embodiments, an anti-CD122 antibody comprising an amino acid sequence of an Fc region of human IgG1 (see Table 15) is not effector null.

Further provided herein is an immunoconjugate comprising an anti-CD122 antibody or an antigen-binding portion thereof, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Examples of suitable therapeutic agents include, but are not limited to, immunomodulatory agents, cytotoxins, radioisotopes, chemotherapeutic agents, anti-angiogenic agents, antiproliferative agents, pro-apoptotic agents, and cytostatic and cytolytic enzymes (for example RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include, but are not limited to, JAK kinase inhibitors, taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumors and immunosuppressive agents that suppress cytokine production, down-regulate self-antigen expression, or mask MHC antigens.

### Pharmaceutical Compositions

The anti-CD122 antibodies and antigen-binding portions provided herein (also referred to herein as “active compounds”) can be incorporated into pharmaceutical compositions suitable for administration. Such compositions typically comprise an anti-CD122 antibody or antigen-binding portion (or an immunoconjugate comprising said antibody or portion), and a pharmaceutically acceptable carrier, diluent or excipient. Such materials should be non-toxic and should not interfere with the efficacy of the anti-CD122 antibody or antigen-binding fragment thereof. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington’s Pharmaceutical Sciences, a standard reference text in the field, which is incorporated herein by reference. Some examples of such carriers or diluents include, but are not limited to, water, saline, Ringer’s solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the anti-CD122 antibody or antigen-binding portion thereof, an increase in its lifespan and/or in its efficacy in the body or an increase in its solubility in solution.

Provided herein is a pharmaceutical composition comprising (i) an anti-CD122 antibody or an antigen-binding portion thereof, wherein the antibody or antigen-binding portion comprises a VH region and a VL region, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; and (ii) a pharmaceutically acceptable carrier, diluent or excipient; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15.

Provided herein is a pharmaceutical composition comprising (i) an anti-CD122 antibody or an antigen-binding portion thereof, wherein the antibody or antigen-binding portion comprises a VH region and a VL region, wherein: (a) the VH region amino acid sequence comprises or consists of SEQ ID NO: 1 and the VL region amino acid sequence comprises or consists of SEQ ID NO: 17; or (b) the VH region amino acid sequence comprises or consists of SEQ ID NO: 1 and the VL region amino acid sequence comprises or consists of SEQ ID NO: 9; and (ii) a pharmaceutically acceptable carrier, diluent or excipient.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the anti-CD122 antibody or antigen-binding portion thereof may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Antibodies

Provided herein is a nucleic acid molecule (e.g., an isolated nucleic acid molecule) encoding an amino acid sequence of an anti-CD122 antibody or anti-CD122 antigen-binding portion described herein (or an amino acid sequence of a (i) VH region, (ii) a VL region, or (iii) both a VH region and a VL region of an antibody or antigen-binding portion). Further provided herein is a nucleic acid molecule (e.g., an isolated nucleic acid molecule) encoding (i) a heavy chain, (ii) a light chain, or (iii) both a heavy chain and a light chain of an anti-CD122 antibody or anti-CD122 antigen-binding portion described herein. In some embodiments, a nucleic acid molecule encoding a VH region, a VL region, a heavy chain or a light chain comprises a signal sequence. In some embodiments, a nucleic acid molecule encoding a VH region, a VL region, a heavy chain or a light chain does not comprise a signal sequence.

In some embodiments, a nucleic acid molecule encodes an amino acid sequence of a VH region and a VL region of an anti-CD122 antibody or an antigen-binding portion thereof, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15. In some embodiments, a nucleic acid molecule further encodes a human framework region amino acid sequence.

In some embodiments, a nucleic acid molecule encodes an amino acid sequence of a VH region and a VL region of an anti-CD122 antibody or an antigen-binding portion thereof, wherein (a) the VH region amino acid sequence comprises or consists of SEQ ID NO: 1 and the VL region amino acid sequence comprises or consists of SEQ ID NO: 17; or (b) the VH region amino acid sequence comprises or consists of SEQ ID NO: 1 and the VL region amino acid sequence comprises or consists of SEQ ID NO: 9.

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a VH region and a VL region of an anti-CD122 antibody or an antigen-binding portion thereof. In some embodiments, a recombinant host cell comprises (i) a first vector or a first nucleic acid molecule encoding a VH region of an anti-CD122 antibody or an antigen-binding portion thereof and (ii) a second vector or a second nucleic acid molecule encoding a VL region of an anti-CD 122 antibody or an antigen-binding portion thereof.

Antibody molecules of the invention, or antigen-binding portion thereof, can be produced using techniques well known in the art, for example, recombinant technologies, phage display technologies, synthetic technologies, computational technologies or combinations of such technologies or other technologies readily known in the art.

Further provided herein is a method for producing an anti-CD122 antibody or an antigen-binding portion thereof, the method comprising: culturing a recombinant host cell comprising an expression vector described herein under conditions whereby the nucleic acid segment is expressed, thereby producing the anti-CD122 antibody or antigen-binding portion. The antibody or antigen-binding portion may then be isolated from the host cell or culture. Anti-CD122 antibodies and antigen-binding portions thereof can be produced by any of a variety of methods known to those skilled in the art. In certain embodiments, anti-CD122 antibodies and antigen-binding portions thereof can be produced recombinantly. For example, nucleic acid sequences encoding one or more of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 18, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, antibody light chain proteins and heavy chain proteins are produced in a cell with a signal sequence that is removed upon production of a mature anti-CD122 antibody or antigen-binding portion thereof.

Those skilled in the art will be able to determine whether an antibody or antigen-binding portion comprising a given polypeptide sequence binds to CD122 protein without undue experimentation using standard methodologies, for example, Western blots, ELISA, and the like.

Provided herein is a method of producing an antibody that specifically binds to human CD122 and optionally also to cynomolgus and/or rhesus monkey CD122, or an antigen-binding portion thereof, comprising the steps of:
(1) grafting anti-CD122 CDRs from a non-human source into a human v-domain framework to produce a humanized anti-CD122 antibody molecule or antigen-binding portion thereof;
(2) generating a library of clones of the humanized anti-CD122 antibody molecule or antigen-binding portion thereof comprising one or more mutations in the CDRs;
(3) screening the library for binding to human CD122 and optionally also to cynomolgus and/or rhesus monkey CD122;
(4) selecting clones from the screening step (3) having binding specificity to human CD122 and optionally also to cynomolgus and/or rhesus monkey CD122, but with reduced or absent binding to human BCAM, human CILP2 or human neudesin; and
(5) producing an antibody molecule which specifically binds to human CD122 and optionally also to cynomolgus and/or rhesus monkey CD122, or an antigen-binding portion thereof from clones selected from step (4).

The method may comprise a further step of producing additional clones based on the clones selected in step (4), for example based on further exploratory mutagenesis at specific positions in the CDRs of the clones selected in step (4), to enhance humanization and/or minimize human T cell epitope content and/or improve manufacturing properties in the antibody molecule or antigen-binding portion thereof produced in step (5).

### Uses of Antibodies

Provided herein are methods and uses of the anti-CD122 antibodies, anti-CD122 antigen-binding portions, immunoconjugates and pharmaceutical compositions described herein for providing a therapeutic benefit to a subject with an immune-mediated disease or disorder.

Provided herein is a method for supressing an immune response in a subject, comprising administering to the subject a therapeutically effective amount of the antibody, the antigen-binding portion, the immunoconjugate or the pharmaceutical composition disclosed herein. Provided herein is a method for supressing an immune response (e.g. an immune response mediated by CD122-positive cells) in a subject, the method comprising administering to the subject a therapeutically effective amount of an anti-CD122 antibody or an antigen-binding portion thereof, wherein the antibody or antigen-binding portion comprises a VH region and a VL region, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15. Provided herein is a method for supressing an immune response in a subject, the method comprising administering to the subject a therapeutically effective amount of an anti-CD122 antibody or an antigen-binding portion thereof, wherein the antibody or antigen-binding portion comprises a VH region and a VL region, wherein: (a) the VH region amino acid sequence comprises or consists of SEQ ID NO: 1 and the VL region amino acid sequence comprises or consists of SEQ ID NO: 17; or (b) the VH region amino acid sequence comprises or consists of SEQ ID NO: 1 and the VL region amino acid sequence comprises or consists of SEQ ID NO: 9. In some embodiments, the immune response is mediated by CD122.

Provided herein is a method for supressing IL-15 induced migration of T cells from skin (e.g., human skin), the method comprising contacting the skin with a therapeutically effective amount of the antibody, the antigen-binding portion, the immunoconjugate or the pharmaceutical composition disclosed herein. Provided herein is a method for supressing IL-15 induced migration of T cells from skin (e.g., human skin), the method comprising contacting the skin with a therapeutically effective amount of an anti-CD122 antibody or an antigen-binding portion thereof, wherein the antibody or antigen-binding portion comprises a VH region and a VL region, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15. Provided herein is a method for supressing IL-15 induced migration of T cells from skin (e.g., human skin), the method comprising contacting the skin with a therapeutically effective amount of an anti-CD122 antibody or an antigen-binding portion thereof, wherein the antibody or antigen-binding portion comprises a VH region and a VL region, wherein: (a) the VH region amino acid sequence comprises or consists of SEQ ID NO: 1 and the VL region amino acid sequence comprises or consists of SEQ ID NO: 17; or (b) the VH region amino acid sequence comprises or consists of SEQ ID NO: 1 and the VL region amino acid sequence comprises or consists of SEQ ID NO: 9. In some embodiments, the T cells are CD8+ T cells. In some embodiments, the T cells are CD4+ T cells. In some embodiments, the skin is skin of a subject having a disease or disorder associated with overexpression of CD122, or expression of CD122 on cells that do not normally express CD122.

An anti-CD122 antibody or antigen-binding portion thereof as described herein may be used in a method of treatment of the human or animal body, including prophylactic or preventative treatment (e.g., treatment before the onset of a condition in a subject to reduce the risk of the condition occurring in the subject; delay its onset; or reduce its severity after onset). The method of treatment may comprise administering the anti-CD122 antibody or antigen-binding portion to a subject in need thereof. Provided herein is a method for treating or preventing a disease in a subject, comprising administering to the subject a therapeutically effective amount of the antibody, the antigen-binding portion, the immunoconjugate or the pharmaceutical composition disclosed herein.

Provided herein is a method for treating or preventing a disease in a subject, the method comprising administering to the subject a therapeutically effective amount of an anti-CD122 antibody or an antigen-binding portion thereof, wherein the antibody or antigen-binding portion comprises a VH region and a VL region, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15. Provided herein is a method for treating or preventing a disease in a subject, the method comprising administering to the subject a therapeutically effective amount of an anti-CD122 antibody or an antigen-binding portion thereof, wherein the antibody or antigen-binding portion comprises a VH region and a VL region, wherein: (a) the VH region amino acid sequence comprises or consists of SEQ ID NO: 1 and the VL region amino acid sequence comprises or consists of SEQ ID NO: 17; or (b) the VH region amino acid sequence comprises or consists of SEQ ID NO: 1 and the VL region amino acid sequence comprises or consists of SEQ ID NO: 9.

Provided herein is a method for ameliorating, treating or reducing the severity of a symptom of a disease in a subject, the method comprising administering to the subject a therapeutically effective amount of an anti-CD122 antibody or an antigen-binding portion thereof, wherein the antibody or antigen-binding portion comprises a VH region and a VL region, wherein: (a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or (b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15. Provided herein is a method for ameliorating, treating or reducing the severity of a symptom of a disease in a subject, the method comprising administering to the subject a therapeutically effective amount of an anti-CD122 antibody or an antigen-binding portion thereof, wherein the antibody or antigen-binding portion comprises a VH region and a VL region, wherein: (a) the VH region amino acid sequence comprises or consists of SEQ ID NO: 1 and the VL region amino acid sequence comprises or consists of SEQ ID NO: 17; or (b) the VH region amino acid sequence comprises or consists of SEQ ID NO: 1 and the VL region amino acid sequence comprises or consists of SEQ ID NO: 9.

In some embodiments, the disease or disorder is associated with overexpression of CD122, or expression of CD122 on cells that do not normally express CD122. In some embodiments, the disease or disorder is mediated by CD122.

In some embodiments, the disease is an inflammatory disease or an autoimmune disease. In some embodiments, the disease is vitiligo, celiac disease, type 1 diabetes, multiple sclerosis, graft-versus-host disease, systemic lupus erythematosus, psoriasis, atopic dermatitis, alopecia areata, ulcerative colitis, or rheumatoid arthritis.

In some embodiments, the VH region, the VL region, or both the VH region and the VL region of an anti-CD122 antibody or antigen-binding portion used in the methods provided herein comprise one or more human framework region amino acid sequences.

As used herein, the term “effective amount” or “therapeutically effective amount” refers to the amount of a pharmaceutical agent, e.g., an anti-CD122 antibody or an antigen-binding portion thereof, which is sufficient to reduce or ameliorate the severity and/or duration of a disease, e.g., vitiligo, celiac disease, type 1 diabetes, multiple sclerosis, graft-versus-host disease, systemic lupus erythematosus, psoriasis, atopic dermatitis, alopecia areata, ulcerative colitis, or rheumatoid arthritis, or one or more symptoms thereof, prevent the advancement of a disease, cause regression of a disease, prevent the recurrence, development, onset or progression of one or more symptoms associated with a disease, detect a disease, or enhance or improve the prophylactic or therapeutic effect(s) of another related therapy (e.g., prophylactic or therapeutic agent) for a CD122-mediated disease.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann J.A. et al., 1991, Int. J. Cancer 47: 659-664; Bagshawe K.D. et al., 1991, Antibody, Immunoconjugates and Radiopharmaceuticals 4: 915-922). Specific dosages may be indicated herein or in the Physician’s Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

A typical antibody dose will be in the range 100 µg to 1 g for systemic applications, and 1 µg to 1 mg for intradermal injection. An initial higher loading dose, followed by one or more lower doses, may be administered. In some embodiments, the antibody is a whole antibody, e.g., the IgG1 or IgG4 isotype. This is a dose for a single treatment of an adult subject, which may be proportionally adjusted for children and infants, and also adjusted for other antibody formats in proportion to molecular weight. Treatments may be repeated at daily, twice-weekly, weekly or monthly intervals, at the discretion of the physician. The treatment schedule for a subject may be dependent on the pharmacokinetic and pharmacodynamic properties of the antibody composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, anti-CD122 antibody molecules and antigen-binding portions as described herein may be administered as sub-cutaneous injections. Sub-cutaneous injections may be administered using an auto-injector, for example for long term prophylaxis/treatment.

In some embodiments, the therapeutic effect of an anti-CD122 antibody or an antigen-binding portion thereof may persist for several half-lives, depending on the dose. For example, the therapeutic effect of a single dose of an anti-CD122 antibody or an antigen-binding portion thereof may persist in a subject for 1 month or more, 2 months or more, 3 months or more, 4 months or more, 5 months or more, or 6 months or more.

In some embodiments, a subject may be treated with an anti-CD122 antibody or an anti-CD122 antigen-binding portion, an immunoconjugate or a pharmaceutical composition described herein and an additional therapeutic agent or therapy that is used to treat a CD122-mediated disease or disorder or a symptom or complication of a CD122-mediated disease or disorder. The anti-CD122 antibody or an anti-CD122 antigen-binding portion and the additional therapeutic agent or therapy may be administered simultaneously or sequentially.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

Further provided herein is an anti-CD122 antibody or an anti-CD122 antigen-binding portion, an immunoconjugate or a pharmaceutical composition described herein, for use in the treatment of a disease or a disorder.

Provided herein is an anti-CD122 antibody or an anti-CD122 antigen-binding portion, an immunoconjugate or a pharmaceutical composition described herein, for use as a medicament.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 October 11) Molecular systems biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥ 0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | -1 | -2 | -2 | 0 | -1 | -1 | 0 | -2 | -1 | -1 | -1 | -1 | -2 | -1 | 1 | 0 | -3 | -2 | 0 |
| R | -1 | 5 | 0 | -2 | -3 | 1 | 0 | -2 | 0 | -3 | -2 | 2 | -1 | -3 | -2 | -1 | -1 | -3 | -2 | -3 |
| N | -2 | 0 | 6 | 1 | -3 | 0 | 0 | 0 | 1 | -3 | -3 | 0 | -2 | -3 | -2 | 1 | 0 | -4 | -2 | -3 |
| D | -2 | -2 | 1 | 6 | -3 | 0 | 2 | -1 | -1 | -3 | -4 | -1 | -3 | -3 | -1 | 0 | -1 | -4 | -3 | -3 |
| C | 0 | -3 | -3 | -3 | 9 | -3 | -4 | -3 | -3 | -1 | -1 | -3 | -1 | -2 | -3 | -1 | -1 | -2 | -2 | -1 |
| Q | -1 | 1 | 0 | 0 | -3 | 5 | 2 | -2 | 0 | -3 | -2 | 1 | 0 | -3 | -1 | 0 | -1 | -2 | -1 | -2 |
| E | -1 | 0 | 0 | 2 | -4 | 2 | 5 | -2 | 0 | -3 | -3 | 1 | -2 | -3 | -1 | 0 | -1 | -3 | -2 | -2 |
| G | 0 | -2 | 0 | -1 | -3 | -2 | -2 | 6 | -2 | -4 | -4 | -2 | -3 | -3 | -2 | 0 | -2 | -2 | -3 | -3 |
| H | -2 | 0 | 1 | -1 | -3 | 0 | 0 | -2 | 8 | -3 | -3 | -1 | -2 | -1 | -2 | -1 | -2 | -2 | 2 | -3 |
| I | -1 | -3 | -3 | -3 | -1 | -3 | -3 | -4 | -3 | 4 | 2 | -3 | 1 | 0 | -3 | -2 | -1 | -3 | -1 | 3 |
| L | -1 | -2 | -3 | -4 | -1 | -2 | -3 | -4 | -3 | 2 | 4 | -2 | 2 | 0 | -3 | -2 | -1 | -2 | -1 | 1 |
| K | -1 | 2 | 0 | -1 | -3 | 1 | 1 | -2 | -1 | -3 | -2 | 5 | -1 | -3 | -1 | 0 | -1 | -3 | -2 | -2 |
| M | -1 | -1 | -2 | -3 | -1 | 0 | -2 | -3 | -2 | 1 | 2 | -1 | 5 | 0 | -2 | -1 | -1 | -1 | -1 | 1 |
| F | -2 | -3 | -3 | -3 | -2 | -3 | -3 | -3 | -1 | 0 | 0 | -3 | 0 | 6 | -4 | -2 | -2 | 1 | 3 | -1 |
| P | -1 | -2 | -2 | -1 | -3 | -1 | -1 | -2 | -2 | -3 | -3 | -1 | -2 | -4 | 7 | -1 | -1 | -4 | -3 | -2 |
| S | 1 | -1 | 1 | 0 | -1 | 0 | 0 | 0 | -1 | -2 | -2 | 0 | -1 | -2 | -1 | 4 | 1 | -3 | -2 | -2 |
| T | 0 | -1 | 0 | -1 | -1 | -1 | -1 | -2 | -2 | -1 | -1 | -1 | -1 | -2 | -1 | 1 | 5 | -2 | -2 | 0 |
| W | -3 | -3 | -4 | -4 | -2 | -2 | -3 | -2 | -2 | -3 | -2 | -3 | -1 | 1 | -4 | -3 | -2 | 11 | 2 | -3 |
| Y | -2 | -2 | -2 | -3 | -2 | -1 | -2 | -3 | 2 | -1 | -1 | -2 | -1 | 3 | -3 | -2 | -2 | 2 | 7 | -1 |
| V | 0 | -3 | -3 | -3 | -1 | -2 | -2 | -3 | -3 | 3 | 1 | -2 | 1 | -1 | -2 | -2 | 0 | -3 | -1 | 4. |

“Antibody-drug conjugate” and “immunoconjugate” refer to an antibody molecule, or antigen-binding portion thereof, including antibody derivatives, that binds to CD122 and is conjugated to cytotoxic, cytostatic and/or therapeutic agents.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule’s antigen-binding regions. Epitopes can consist of defined regions of primary secondary or tertiary protein structure and includes combinations of secondary structural units or structural domains of the target recognized by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example, nuclear magnetic resonance spectroscopy).

The term “potency” is a measurement of biological activity and may be designated as IC₅₀, EC₅₀, or effective concentration of an antibody or antibody drug conjugate to the antigen CD122 to inhibit 50% of activity measured in a CD122 activity assay as described herein.

The term “inhibit” or “neutralize” as used herein with respect to bioactivity of an antibody disclosed herein means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to CD122.

In the present description, any concentration range, percentage range, ratio range, or integer range is to be understood to include the value of any integer within the recited range and, when appropriate, fractions thereof (such as one tenth and one hundredth of an integer), unless otherwise indicated. The use of the alternative (e.g., “or”) should be understood to mean either one, both, or any combination thereof of the alternatives. As used herein, the terms “include” and “comprise” are used synonymously.

The section headings used herein are for organizational purposes only and are not to be construed as limiting the subject matter described.

All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term’s definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

Any of the aspects and embodiments described herein can be combined with any other aspect or embodiment as disclosed here in the Summary, in the Drawings, and/or in the Detailed Description, including the below specific, non-limiting, examples/embodiments of the present disclosure.

### NUMBERED EMBODIMENTS

Notwithstanding the appended claims, the disclosure sets forth the following numbered embodiments:

1. An anti-CD122 antibody or an antigen-binding portion thereof, wherein the antibody or antigen-binding portion comprises a heavy chain variable (VH) region and a light chain variable (VL) region wherein:
(a) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 18, a LCDR2 comprising SEQ ID NO: 13 and a LCDR3 comprising SEQ ID NO: 15; or
(b) the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15.

2. The antibody or antigen-binding portion of embodiment 1 wherein
(a) the VH region amino acid sequence comprises SEQ ID NO: 1 and the VL region amino acid sequence comprises SEQ ID NO: 17; or
(b) the VH region amino acid sequence comprises SEQ ID NO: 1 and the VL region amino acid sequence comprises SEQ ID NO: 9.

3. The antibody or antigen-binding portion of embodiment 1 or 2, wherein the antibody or antigen-binding portion is humanized or chimeric.

4. The antibody or antigen-binding portion of any one of embodiments 1-3, wherein the VH region, the VL region, or both the VH and the VL region comprise one or more human framework region amino acid sequences.

5. The antibody or antigen-binding portion of any one of embodiments 1-4, wherein the VH region, the VL region, or both the VH and the VL region comprise a human variable region framework scaffold amino acid sequence into which the CDR amino acid sequences have been inserted.

6. The antibody or antigen-binding portion of any one of embodiments 1 and 3-5, wherein the VH region comprises an IGHV3-23 human germline scaffold amino acid sequence into which the HCDR1, HCDR2 and HCDR3 amino acid sequences have been inserted.

7. The antibody or antigen-binding portion of any one of embodiments 1 and 3-6, wherein the VL region comprises an IGKV1-33 human germline scaffold amino acid sequence into which the LCDR1, LCDR2 and LCDR3 amino acid sequences have been inserted.

8. The antibody or antigen-binding portion of any one of embodiments 1-7, wherein the antibody comprises an immunoglobulin constant region.

9. The antibody or antigen-binding portion of embodiment 8, wherein the immunoglobulin constant region is IgG, IgE, IgM, IgD, IgA or IgY.

10. The antibody or antigen-binding portion of embodiment 9, wherein the immunoglobulin constant region is IgG1, IgG2, IgG3, IgG4, IgA1 or IgA2.

11. The antibody or antigen-binding portion of embodiment 8, wherein the immunoglobulin constant region is immunologically inert.

12. The antibody or antigen-binding portion of embodiment 8, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A, L235A and G237A or a wild-type human IgG2 constant region, wherein numbering is according to the EU index as in Kabat.

13. The antibody or antigen-binding portion of embodiment 8, wherein the immunoglobulin constant region comprises any one of SEQ ID NOs: 32-38.

14. The antibody or antigen-binding portion of any one of embodiments 1-13, wherein the antibody or antigen-binding portion is an Fab, an Fab′, an F(ab′)₂, an Fv, an scFv, a maxibody, a minibody, a diabody, a triabody, a tetrabody, or a bis-scFv.

15. The antibody or antigen-binding portion of any one of embodiments 1-14, wherein the antibody is monoclonal.

16. The antibody or antigen-binding portion of any one of embodiments 1-15, wherein the antibody is a tetrameric antibody, a tetravalent antibody or a multispecific antibody.

17. The antibody or antigen-binding portion of any one of embodiments 1-16, wherein the antibody is a bispecific antibody that binds specifically to a first antigen and a second antigen, wherein the first antigen is CD122 and the second antigen is not CD122.

18. An immunoconjugate comprising the antibody or antigen-binding portion of any one of embodiments 1-17, linked to a therapeutic agent.

19. The immunoconjugate of embodiment 18, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

20. A pharmaceutical composition comprising the antibody or antigen-binding portion of any one of embodiments 1-17 or the immunoconjugate of embodiment 18 or 19, and a pharmaceutically acceptable carrier, diluent or excipient.

21. A nucleic acid molecule encoding
(a) the VH region amino acid sequence;
(b) the VL region amino acid sequence; or
(c) both the VH and the VL region amino acid sequences
of the antibody or antigen-binding portion of any one of embodiments 1-17.

22. An expression vector comprising the nucleic acid molecule of embodiment 21.

23. A recombinant host cell comprising the nucleic acid molecule of embodiment 21 or the expression vector of embodiment 22.

24. A method of producing an anti-CD122 antibody or an antigen-binding portion thereof, the method comprising: culturing a recombinant host cell comprising the expression vector of embodiment 22 under conditions whereby the nucleic acid molecule is expressed, thereby producing the antibody or antigen-binding portion; and isolating the antibody or antigen-binding portion from the host cell or culture.

25. A method for supressing an immune response in a subject, comprising administering to the subject a therapeutically effective amount of the antibody or antigen-binding portion of any one of embodiments 1-17, the immunoconjugate of embodiment 18 or 19 or the pharmaceutical composition of embodiment 20.

26. The method of embodiment 25, wherein the immune response is mediated by CD122.

27. A method for treating or preventing a disease in a subject, comprising administering to the subject a therapeutically effective amount of the antibody or antigen-binding portion of any one of embodiments 1-17, the immunoconjugate of embodiment 18 or 19 or the pharmaceutical composition of embodiment 20.

28. The method of embodiment 27, wherein the disease is an inflammatory disease or an autoimmune disease.

29. The method of embodiment 27, wherein the disease is vitiligo, celiac disease, type 1 diabetes, multiple sclerosis, graft-versus-host disease, systemic lupus erythematosus, psoriasis, atopic dermatitis, alopecia areata, ulcerative colitis, or rheumatoid arthritis.

30. A method for supressing IL-15 induced migration of T cells from skin, the method comprising contacting the skin with a therapeutically effective amount of the antibody or antigen-binding portion of any one of embodiments 1-17, the immunoconjugate of embodiment 18 or 19 or the pharmaceutical composition of embodiment 20.

31. The antibody or antigen-binding portion of any one of embodiments 1-17, the immunoconjugate of embodiment 18 or 19 or the pharmaceutical composition of embodiment 20, for use as a medicament.

The disclosure will be further clarified by the following examples, which are intended to be purely exemplary of the disclosure and in no way limiting.

### EXAMPLES

### Example 1. Generation of Optimized Anti-CD122 Therapeutic Antibodies

### Introduction

In this example, we successfully generate a panel of antagonistic, optimized anti-CD122 antibodies. These anti-CD122 antibodies are well expressed, biophysically stable, highly soluble and of maximized identity to preferred human germlines.

### Materials and Methods

### Antibody V-domain Specificity Testing: Human Receptor Array Analyses

Human cell membrane receptor proteome arrays were performed at Retrogenix Ltd. Primary screens: 5 µg/ml of IgG1-MIKβ1 (humanized, also referred to as VillMab-1) antibody was screened for binding against fixed HEK293 cells/slides expressing 4975 human plasma membrane proteins individually (14 slide sets, n=2 slides per slide set). All transfection efficiencies exceeded the minimum threshold. Antibody binding was detected using AF647 fluorescent secondary anti-human IgG1 antibody. Primary hits (duplicate spots) were identified by analysing fluorescence (AF647 and ZsGreen1) on ImageQuant. Vectors encoding all hits were sequenced to confirm their correct identities.

Confirmation/specificity screens: Vectors encoding all hits, plus control vectors encoding MS4A1 (CD20) and EGFR, were spotted in duplicate on new slides, and used to reverse transfect human HEK293 cells as before. All transfection efficiencies exceeded the minimum threshold. Identical fixed slides were treated with 5 µg/ml of each test antibody, 5 µg/ml of the negative control antibody, 1 µg/ml Rituximab biosimilar (positive control), Isotype IgG1 (Ab00102 human IgG1 anti-Fluorescein) or no test molecule (secondary only; negative control) (n=2 slides per treatment). Slides were analyzed as above.

Flow cytometry confirmation screen: Expression vectors encoding ZsGreen1 only, or ZsGreen1 and CD122, BCAM, were transfected into human HEK293 cells. Each live transfectant was incubated with 1 and 5 mg/ml of each of the test antibodies and the Isotype control antibody. Cells were washed and incubated with the same AF647 anti-human IgG Fc detection antibody as used in the cell microarray screens. Cells were again washed and analysed by flow cytometry using an Accuri flow cytometer (BD). A 7AAD live/dead dye was used to exclude dead cells, and ZsGreen1-positive cells (i.e. transfected cells) were selected for analyses.

### CD122 Library Generation and Selection

The CD122 Fab repertoire was assembled by mass oligo synthesis and PCR. The amplified Fab repertoire was then cloned via restriction-ligation into a phagemid vector, transformed into E.coli TG-1 cells, and the phage repertoire rescued essentially as previously described in detail (Finlay et al., 2011, Methods Mol Biol 681: 383-401). Phage selections were performed by coating streptavidin magnetic microbeads with biotinylated CD122 target protein (either human or cynomolgus), washing the beads thrice with PBS and resuspending in PBS pH7.4 plus 5% skim milk protein. These beads were coated at 100 nM target protein in round 1 of selection, followed by reduced antigen concentrations in three successive rounds. In each round, phage were eluted using trypsin before re-infection into TG1 cells.

### Fab and IgG Expression and Purification

Mammalian codon-optimized synthetic genes encoding the heavy and light chain variable domains of the lead panel anti-CD122 antibodies plus the MIKβ1 variants were cloned into mammalian expression vectors comprising effector function null human IgG1 (‘IgG1-3M’; human IgG1 containing L234A, L235A, G237A mutations in the lower hinge that abrogate normal immunoglobulin ADCC, ADCP and CDC functions) and human Cκ domains, respectively. Co-transfection of heavy and light chain containing vector in mammalian expression system was performed, followed by protein A-based purification of the IgG, quantification and QC on denaturing and non-denaturing SDS-PAGE.

### Direct Binding ELISA for Fab and IgG

Binding and cross-reactivity of the lead panel to the recombinant proteins was initially assessed by binding ELISA. The human CD122 human Fc tagged recombinant protein and the cynomolgus and/or rhesus monkey CD122 human Fc tagged recombinant protein were coated to the surface of MaxiSorp™ flat-bottom 96 well plate at 1 µg/ml. The purified Fab or IgG samples were titrated in two-fold serial dilutions starting from 500 nM to 0.98 nM and allowed to bind to the coated antigens. The Fabs were detected using mouse anti-c-myc antibody followed by donkey anti-mouse IgG conjugated to horseradish peroxidase. The IgGs were detected using the mouse anti-human IgG conjugated to horseradish peroxidase. Binding signals were visualized with 3,3′,5,5′-Tetramethylbenzidine Substrate Solution (TMB) and the absorbance measured at 450 nm.

### BIACORE® Analyses of Fab Affinity for Human and Rhesus CD122

Affinity (KD) of purified IgGs was determined via SPR with antigen in-solution on a BIACORE® 3000 (GE). A mouse anti-human antibody (CH1 specific) was immobilized on a CM5 Sensor Chip to a level of 2000 RU in acetate buffer at pH 4.5 using amine coupling following the Wizard instructions for two channels. One channel was used for background signal correction. The standard running buffer HBS-EP pH 7.4 was used. Regeneration was performed with a single injection of 10 µl of 10 mM Glycine at pH 1.5 at 20 µl/minute. IgG samples were injected for 2 minutes at 50 nM at 30 µl/min followed by and off-rate of 60 seconds. The monomeric antigen (human CD122 His tagged or cynomolgus and/or rhesus monkey CD122 His tag) was injected in two fold serial dilutions from 100 nM down to 3.1 nM, for 2 minutes at 30 µl/min followed by an off-rate of 300 seconds. The obtained sensorgrams were analyzed using the BIACORE® 3000 evaluation (BIAevaluation) software. The KD was calculated by simultaneous fitting of the association and dissociation phases to a 1:1 Langmuir binding model.

### Flow Cytometry of IgGs

Purified IgGs were tested in FACs for binding to human and rhesus CD122 expressed on CHO-K1 stable cell lines and CHO-K1 wild-type cells. The IgG samples were titrated in three-fold serial dilutions starting at 500 nM to 0.98 nM. Binding of IgGs was detected with a mouse anti-human IgG conjugated to FITC. Results were analyzed by examining the Mean Fluorescence Intensity (MFI) of 10000 cells per sample in the BL-1 channel detector of a flow cytometer (AttuneTM NxT Acoustic Focusing Cytometer, Invitrogen/ ThermoFisher Scientific).

### M07e Cell-Based Assay

M07e cells were obtained from DSMZ-German collection of microorganisms and cell cultures and maintained in RPMI supplemented with 10% FBS, 10 ng/mL GM-CSF (Peprotech) and L-glutamine (Corning) according to the guidelines provided by distributor. On day 1, cells were washed in RPMI and resuspended at a density of 2.5 × 10⁵ cells/mL in RPMI supplemented with 10% FBS and L-glutamine (Corning). A total of 5.0×10⁴ cells in a final volume of 200µL were cultured in the wells of a 96 well flat bottom plate for 72 hours at 37° C. in the presence of 50 ng/mL recombinant human IL15 (rhIL15) (R&D) or rhIL15 with antibody. After 72 hours, cells were incubated with 20 µL of WST-1 cell proliferation reagent (Miltenyi) for 3 hours at 37° C. Quantification of cell proliferation was performed with a scanning multi-well spectrophotometer and the measured absorbance at 450 nM was correlated to the number of viable cells.

### Human NK Cell-Based Assay

Human peripheral blood mononuclear cells (PBMCs) were isolated from the whole blood of donors using density gradient centrifugation with Ficoll Histopaque and NK cells were enriched from isolated PBMCs using a Miltenyi Biotec (Bergisch Gladbach, Germany) human NK Cell Isolation Kit according to the manufacturer’s instructions. NK cells were stained with the CellTrace™ CFSE Cell Proliferation Kit according to the manufacturer’s instructions and resuspended in RPMI supplemented with 10% FBS and Penicillin-Streptomycin (Gibco). A total of 10⁵ NK cells were cultured in the wells of a 96 well round bottom plate for 120 hours at 37° C. in the presence of 20 ng/mL recombinant human IL15 (rhIL15) (R&D) or rhIL15 with antibody. After 120 hours, NK cells were washed and stained with anti-human CD3 (UCHT1), CD56 (5.1H11), CD16 (3G8) (1:20 dillution, Biolegend) and CFSE dilution was analyzed with a BD LSR II flow cytometer (BD Biosciences) and FlowJo (Tree Star Inc.).

### NSG-IL15 Mouse Model

Humanized mice were generated by engrafting NOD scid gamma mice that express human IL15 (NSG-Tg) with human hematopoietic stem cells (HSCs). 6 to 8-week-old NSG-Tg mice received 200 cGy of irradiation prior to injection with 10⁵ CD34+ HSC derived from umbilical cord blood. HSC-engrafted NSG-Tg mice were screened at 12 and 16 weeks to determine baseline engraftment in blood. NSG-Tg mice with more than 20% human CD45+ cells that are more than 2% of CD56+, were selected for antibody treatment. Mice were treated with intraperitoneal (i.p.) injections twice weekly (Monday/Thursday schedule) for 3 weeks. Human immune cell levels were quantified in the blood using flow cytometry at 1 and 3 weeks after initiation of treatment, and then at 1, 3, and 5 weeks post treatment. At 5 weeks post treatment mice were euthanized and human immune cell levels in the spleen and blood were measured by flow cytometry. Cells from all tissues were stained with anti-human CD45, CD3, CD4, CD8, CD7, CD56, CD16, Mik-b2, and Mik-b3 (1:20 dillution, Biolegend) and analyzed with a BD LSR II flow cytometer (BD Biosciences) and FlowJo (Tree Star Inc.).

### Results and Discussion

### Pharmacological Modelling: Assessing Feasibility of IL15Rβ Antagonist Antibodies for the Treatment of Diseases in the Skin

In silico modelling was performed to define the pharmacological parameters and characteristics that might drive therapeutic success for an anti-CD122 IgG1 antibody designed to maximise efficacy in specific tissues such as skin. These analyses were based on established values for CD122 target biology (Table 1) and the known potential drug characteristics and dosing parameters for a bivalent IgG antibody (Table 2).

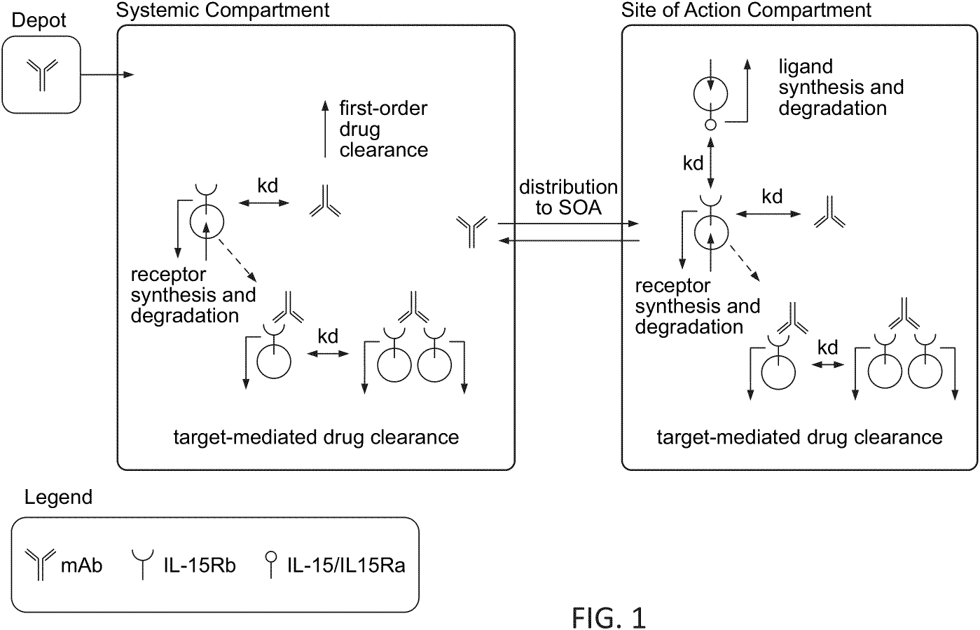

The parameters outlined above were then analyzed to sample their influences on drug potency and distribution, as outlined in FIG. 1. A bottom-up estimate approach was corroborated using reported PK data from t cell leukaemia patients previously dosed with the humanized anti-CD122 IgG1 antibody ‘MIKβ1’. These analyses led to a series of findings relating to IV and/or SC dosing, and antibody CD122 binding affinity, in a human subject of average body mass:

1. At nominal drug affinity of 10 nM KD, sustained inhibition of IL15Rβ (>90%) in the skin was predicted to be feasible with 700 mg IV Q4W or 100 mg SC Q1W (Table 3, Table 4).

2. Higher functional affinity than 10 nM KD can decrease dosing requirements in IV and SC, with KD values closer to 1 nM allowing 99% target occupancy in skin at the maximum dose. This pattern is qualitatively similar for IV and SC dosing routes (Table 3, Table 4).

3. At the nominal drug binding affinity of 10 nM, 100 mg SC Q1W is sufficient to sustain 90% receptor occupancy in the skin, but 100 mg SC Q 1W is not sufficient to sustain >95% receptor occupancy. Higher affinity would be necessary to achieve >95% receptor occupancy at a 100 mg SC Q1W dosing schedule (Table 4).

4. Eliminating systemic target sink has minimal effect on dose required to achieve high (>90%) target occupancy in the skin. Simulations of IV Q4W dosing in the presence of systemic CD122 at known levels, versus in the absence of CD122, suggest that total dose must overcome the systemic target burden, to have optimal activity in skin and drug distribution effects (Table 5).

In synopsis, these analyses suggested an optimal anti-CD122 antagonistic therapeutic antibody would have a functional affinity for CD122 of > 10 nM, allowing dosing at 700 mg IV Q4W or 100 mg SC Q1W. Increased affinity allows greater target coverage at lower doses.

### Antibody Binding Specificity Analyses

In early clinical trials, the humanized anti-CD122 IgG1 antibody “MIKβ1” has been reported to exhibit evidence of accelerated clearance. Accelerated clearance is a risk factor for not being able to achieve the ideal drug characteristics outlined above, as target-mediated drug distribution (TMDD) effects can negatively impact potency. We hypothesized that MIKβ1 might not bind solely to CD122 but might also bind to unidentified and unpredictable human proteins. To examine this possibility, in vitro technologies (Retrogenix, Ltd.), which are based on using high-density arrays of cells expressing >5500 unique human membrane receptors and membrane-tethered secreted proteins, were used to screen for off-target binding specificities in humanized IgGl-MIKβ1 (VillMab-1). This receptor array binding screen identified that VillMab-1 exhibited strong binding to membrane-expressed CD122, but also had potential off-target binding specificity for BCAM (also known as AU, CD239, LU, MSK19, basal cell adhesion molecule (Lutheran blood group)). BCAM is a widely-expressed membrane adhesion protein that could cause reduced PK and exacerbate ‘sink’ effects in the therapeutic dosing of an anti-CD122 antibody.

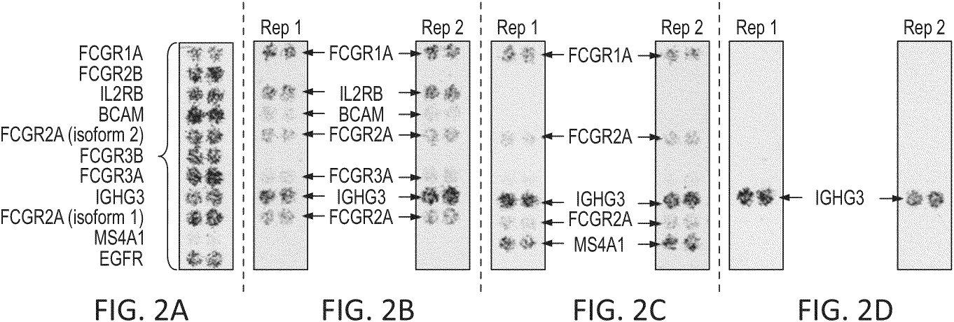

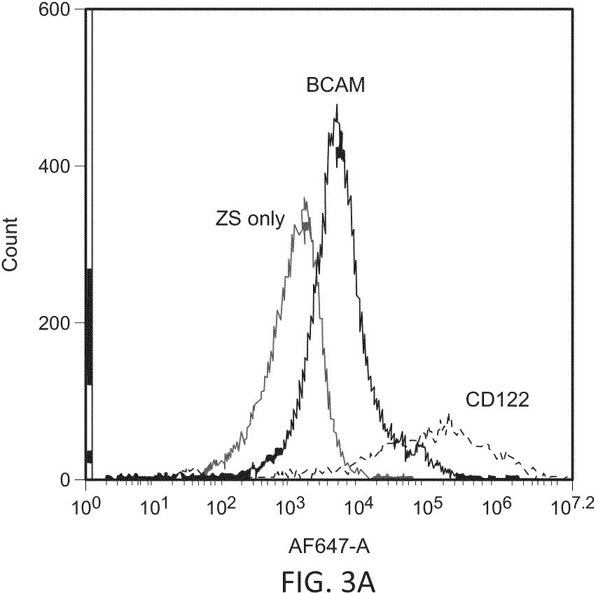

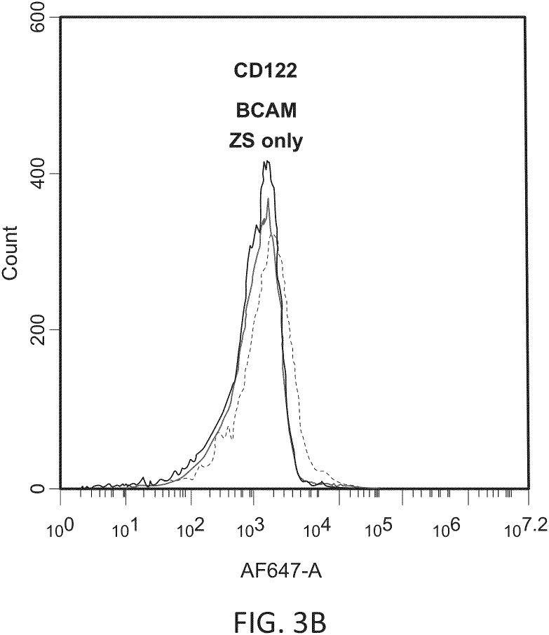

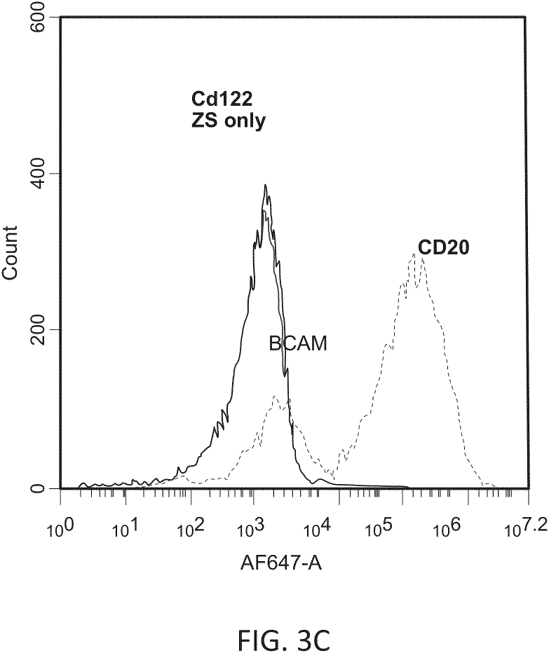

To confirm this off-target binding event, the plasmid encoding for BCAM and control proteins were submitted for DNA sequencing. These analyses confirmed that the encoded proteins were indeed the correct sequences. The plasmid samples for control and potential target receptors were then re-arrayed onto new chips for repeat analyses in duplicate. The effective induction of expression from all re-arrayed plasmids was confirmed by scanning the chips for ZS green, which is co-encoded on all expression plasmids as an internal control marker. This analysis showed clearly detectable ZS expression in all positions where plasmids were spotted (FIG. 2A). Further, identically-spotted slides were then used to re-probe transfected cells with VillMab-1 (FIG. 2B), Rituximab (IgG1 positive control, FIG. 2C), and a chip where no primary antibody probe was applied (FIG. 2D). These analyses showed that VillMab-1 again demonstrated measurable binding over background (on both chips) on cells transfected with BCAM (FIG. 2B). Rituximab demonstrated binding to CD20 as expected, with no observable binding to any other proteins (FIG. 2C). In the chips probed with no primary antibody (FIG. 2D), only the expected control proteins showed any signal. This clean performance of the control chips confirmed that VillMab-1 binding signals on CD122 and BCAM were specific. To further confirm these off-target binding findings, the sequence-verified plasmids were again transfected into HEK-293 cells and binding investigated via flow cytometry (FIG. 3). In this experiment, VillMab-1 showed clear binding to both CD122 and BCAM-transfected cells but no background binding to cells transfected with ZS (“ZS only”, FIG. 3A). Control experiments using the same cells but no primary antibody (FIG. . 3B) sowed that BCAM signals were antibody-related and staining with Rituximab IgG1 anti-CD20 (FIG. 3C) showed binding signal only on CD20-transfected cells, proving that the signal on BCAM is specifically mediated by the binding domains of VillMab-1.

Therapeutic antibodies should ideally have exquisite specificity for their desired target, as off-target binding has been shown to have potential negative effects on the PK, biodistribution and toxicity profiles of IgGs. To address this issue in VillMab-1, exploratory modulation of the VillMab-1 binding interface was caried out as below.

### VillMab-1 Mutagenesis and Paratope Modulation

To bias our engineering efforts towards final lead therapeutic IgG compounds with optimal drug-like properties, we chose to examine mutagenesis-derived variants of the VillMab-1 antibody. Sequence analysis of the v domains of VillMab-1 showed that the original humanization process had used scaffolds related to human germline frameworks IGHV3-23 and IGKV 1-33, which are known to have good solubility and drug development qualities, and are used at high frequency in the expressed human antibody repertoire (Table 6). Despite this use of well-known scaffolds, the frameworks of the variable domains both contained significant numbers of deviations from the germline sequence. In addition, the CDR sequences also contained many residues that differed from the human germlines (Table 6).

The v-domain sequences of VillMab-1 were combined into a Fab phage display format and separate mutagenesis library cassettes were generated for the VH and VL domains by oligo synthesis and assembly. Each mutagenesis cassette encoded for the VillMab-1 residue, the human germline residue, or a homologous amino acid at every position underlined in Table 6. Separate Fab libraries were generated combining the mutation cassette for the VL with the VillMab-1 VH or the VillMab-1 VL with the mutagenesis cassette for the VH. Each final Fab library was ligated into a phage display vector and transformed into E. coli via electroporation to generate > 10⁷ independent clones. Library build quality was verified by sequencing 96 clones per library. This sequencing data showed that the mutated positions effectively sampled the designed diversity. Libraries were rescued using helper phage M13 and selections performed on biotinylated human and cynomolgus and/or rhesus monkey CD122-Fc proteins in multiple separate branches. After round 1 of selection, the preselected mutated VH and VL combinations were used to create a third, combinatorial, library that sampled the selected variability in both V domains simultaneously.

Post-selection periplasmic preparation screening and DNA sequencing revealed the presence of 64 unique, human and rhesus CD122-binding Fab clones that exhibited strong binding to human and rhesus CD122 in ELISA and >50% inhibition of VillMab-1 binding to human and rhesus CD122 in Alphascreen assay. From these unique, library-derived leads, the 15 top clones were identified based on strength of assay signals, level of mutation towards human germline and absence of major developmental liability/chemical degradation motifs (Table 7). This analysis also identified a series of unique sequences in each CDR (Table 8). These unique CDR sequence profiles were used to design 15 further clones (MAB01-MAB15) with potential CDR combinations not found amongst the top 15 library derived clones, as outlined in (Table 9). These 30 unique clones in total were expressed in human IgG1 format in CHO transient culture, purified via Protein A and monomericity >95% confirmed by Size Exclusion Chromatography.

### Lead IgG Specificity and Potency Characteristics

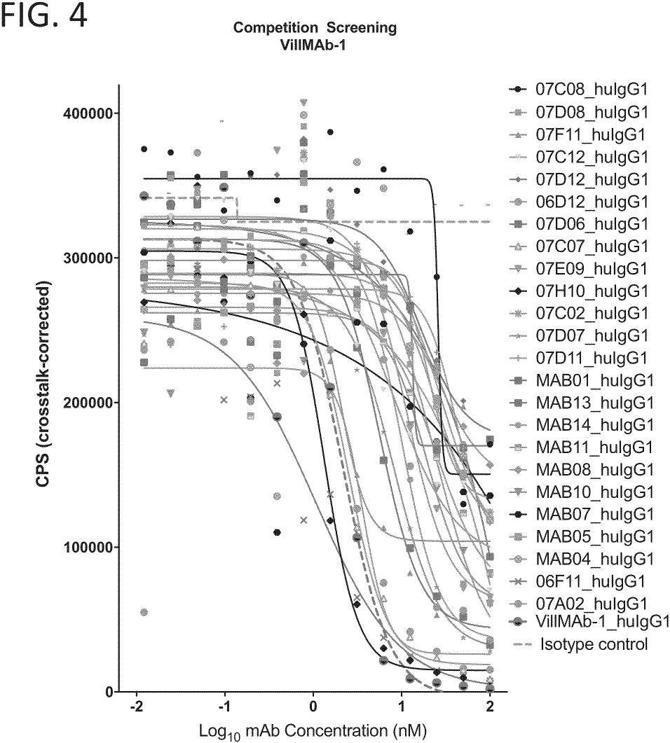

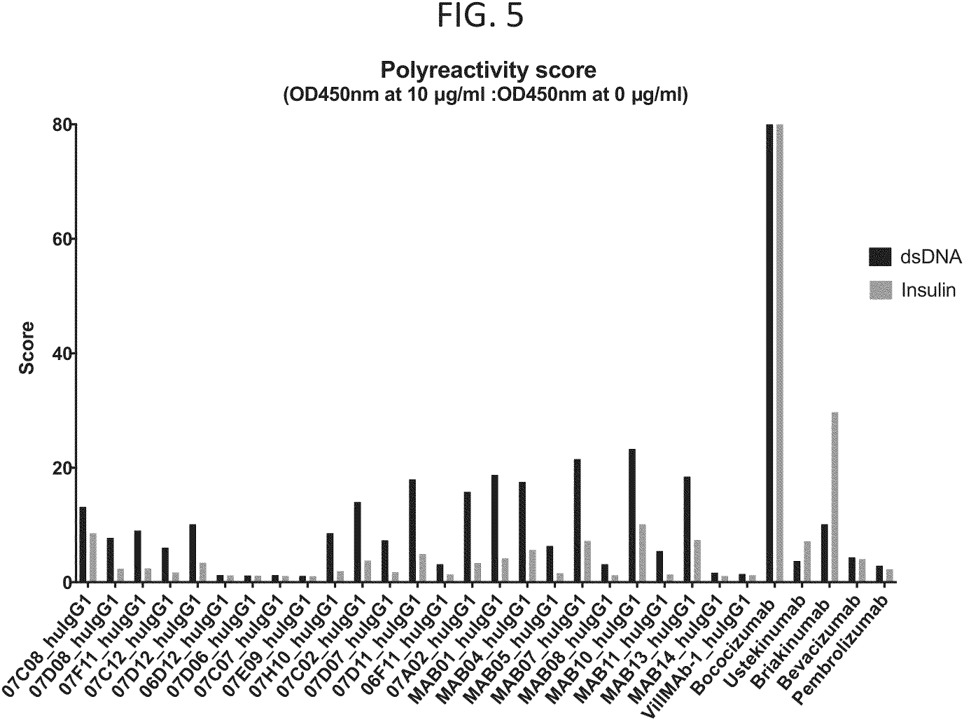

The purified IgGs described above were then tested for competition for the VillMAb-1 binding epitope on human CD122-Fc in Alphascreen format. This analysis showed that 24 out of 30 clones reduced the binding of VillMab-1 to CD122 in a concentration-dependent manner, proving that they retained the functional epitope of the parental antibody (FIG. 4). These 24 clones were then examined in polyreactivity assays to ensure that the final lead clones from initial engineering did not have DNA or insulin binding profiles that are strongly associated with short PK (FIG. 5). This analysis showed that while all 24 clones exhibited signals significantly lower than the positive control Bococizumab, a subset generated particularly low signals that were equivalent to, or improved over, the negative control antibodies Bevacizumab, Ustekinumab and Pembrolizumab (FIG. 5). These findings allowed the prioritization of 6 key leads for further analysis.

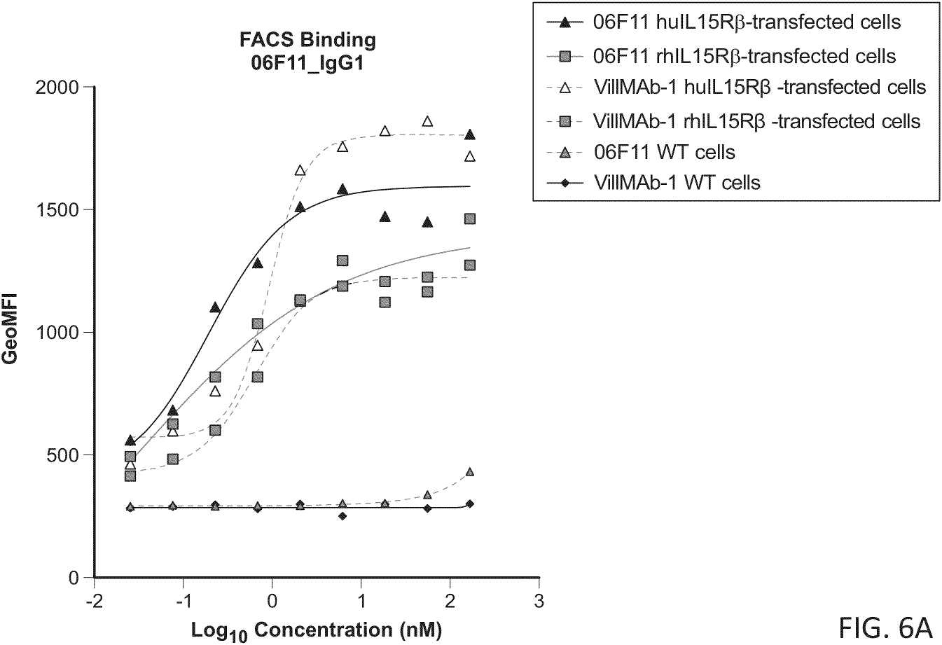

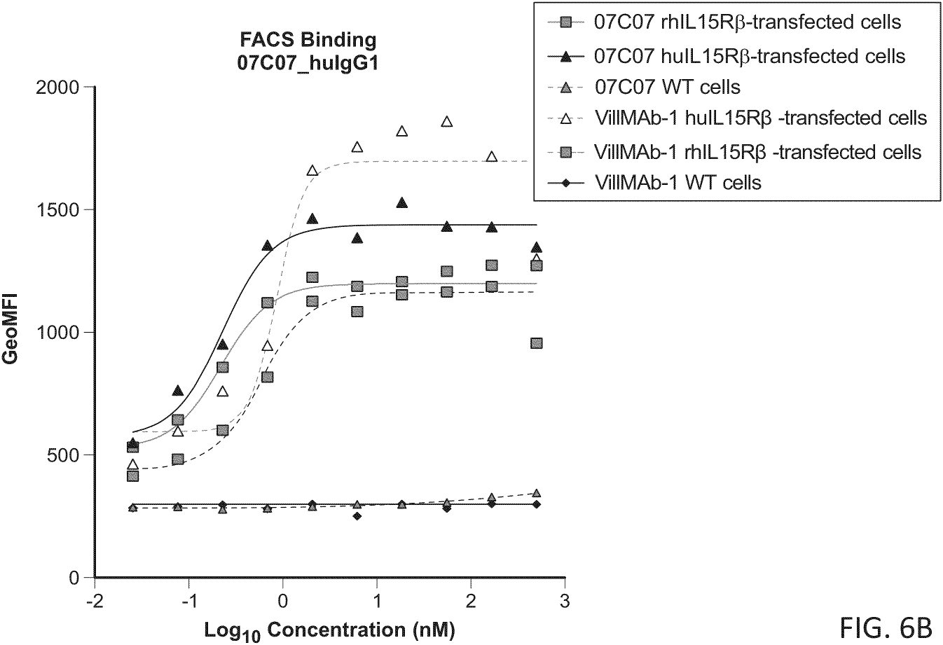

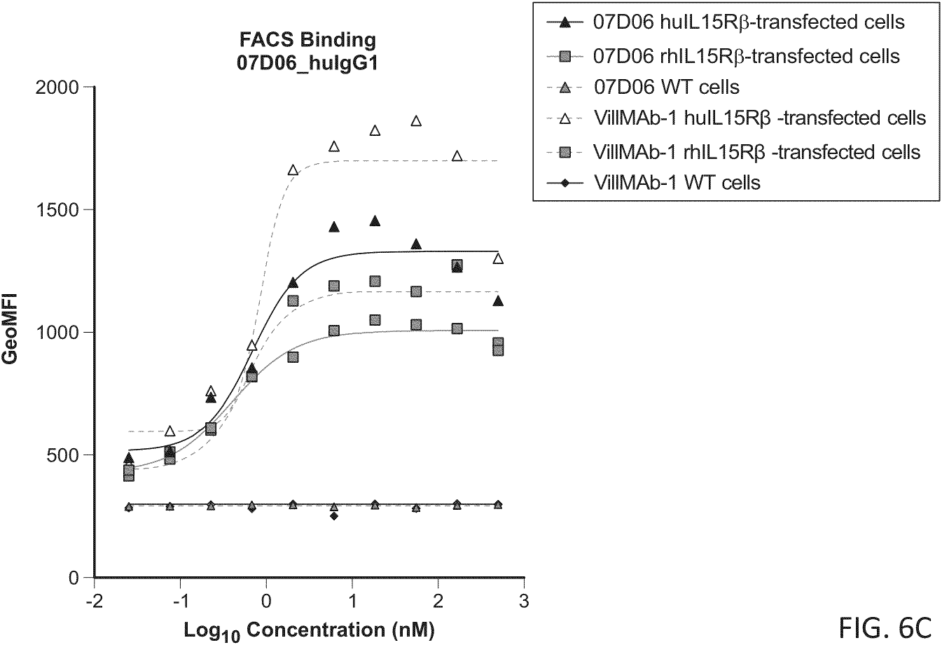

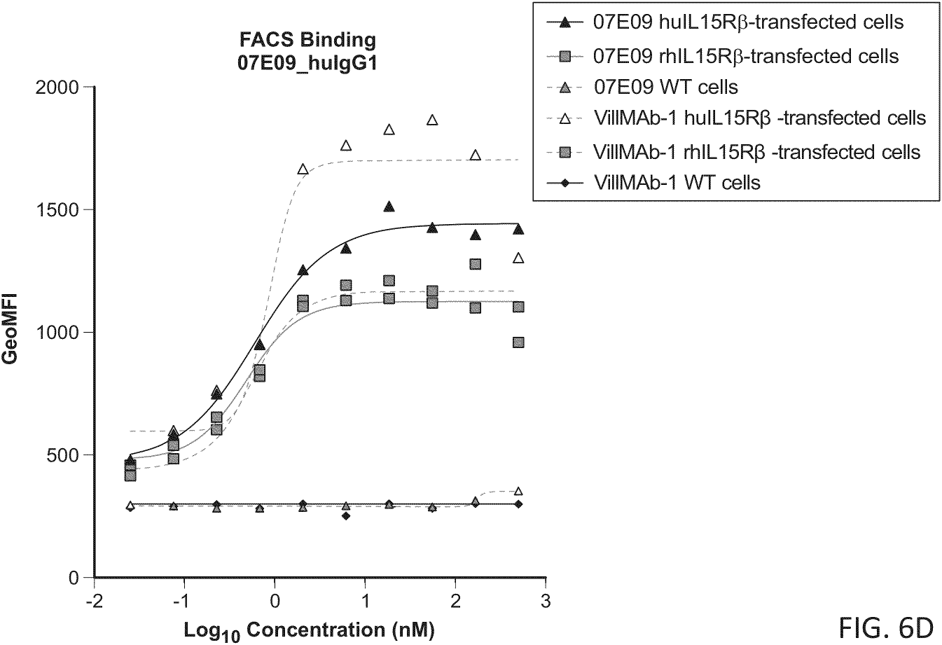

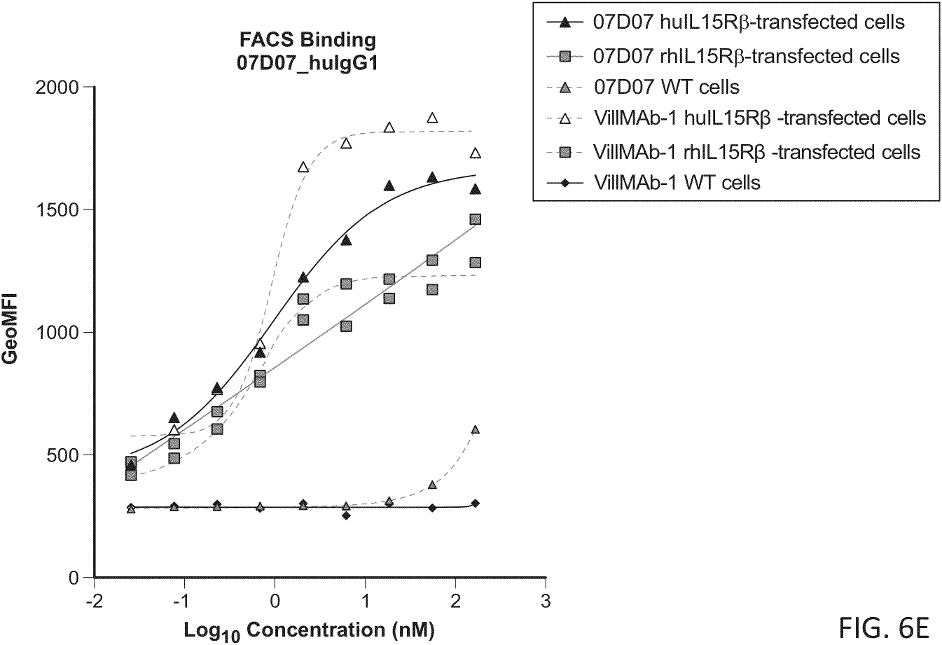

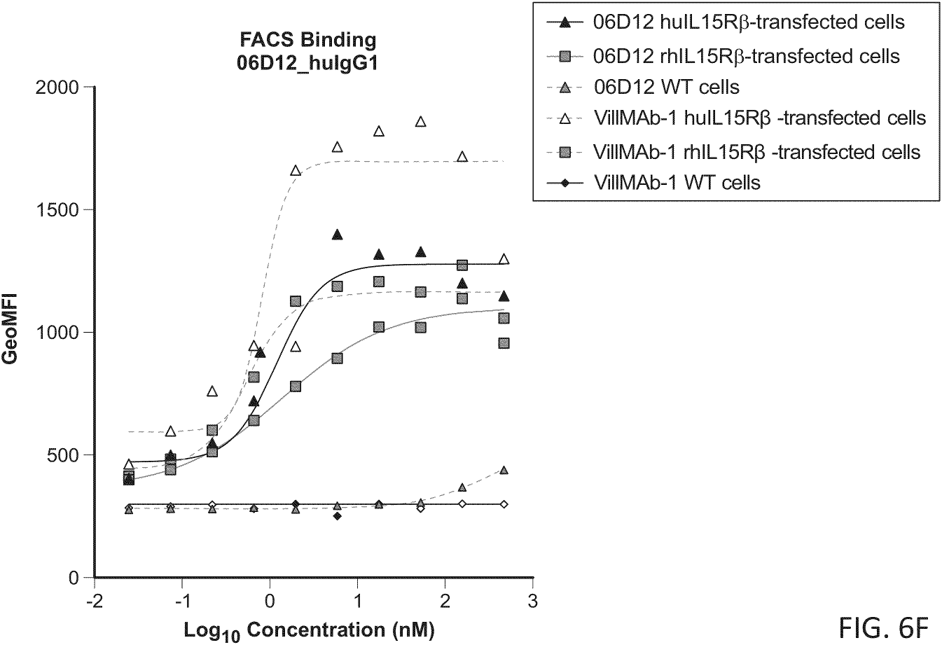

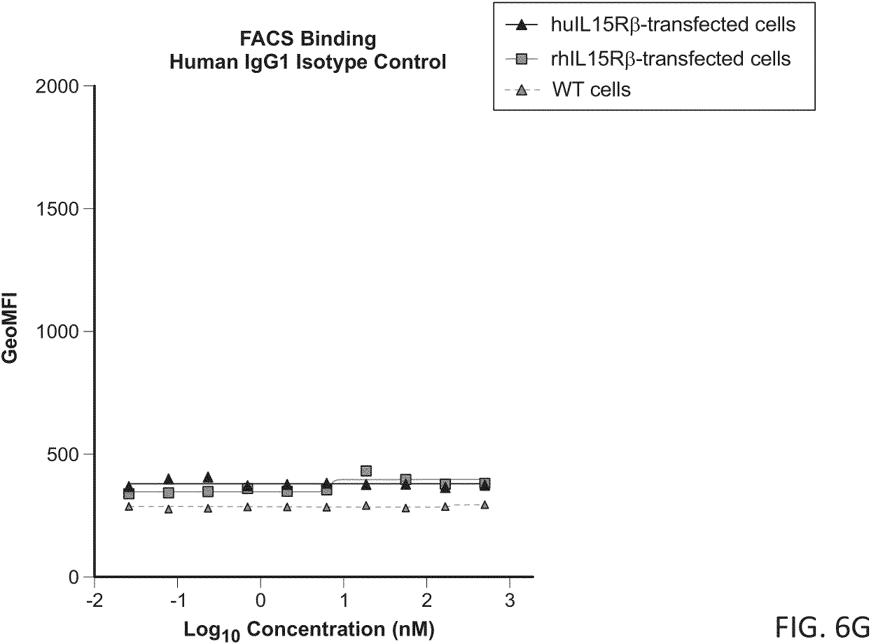

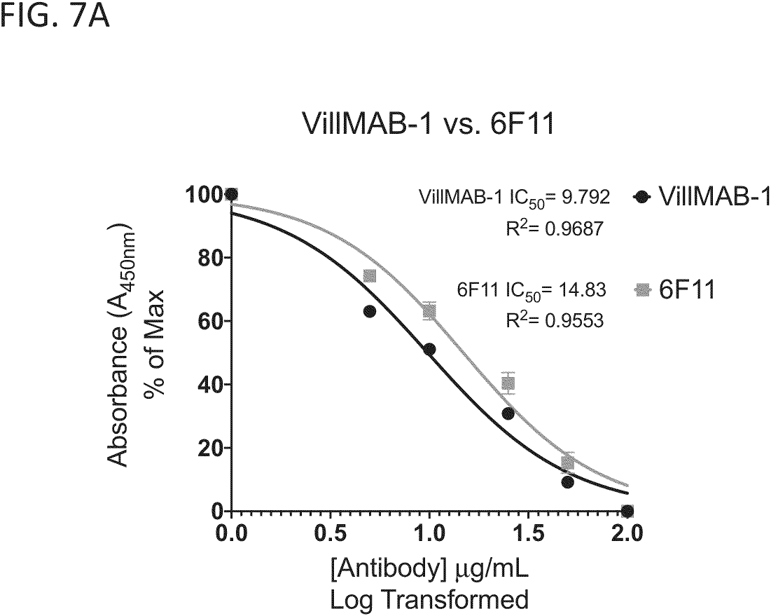

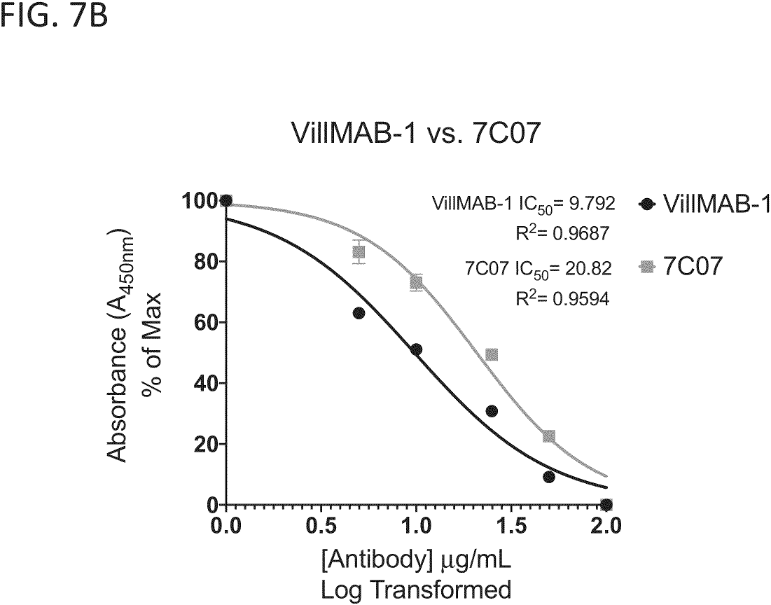

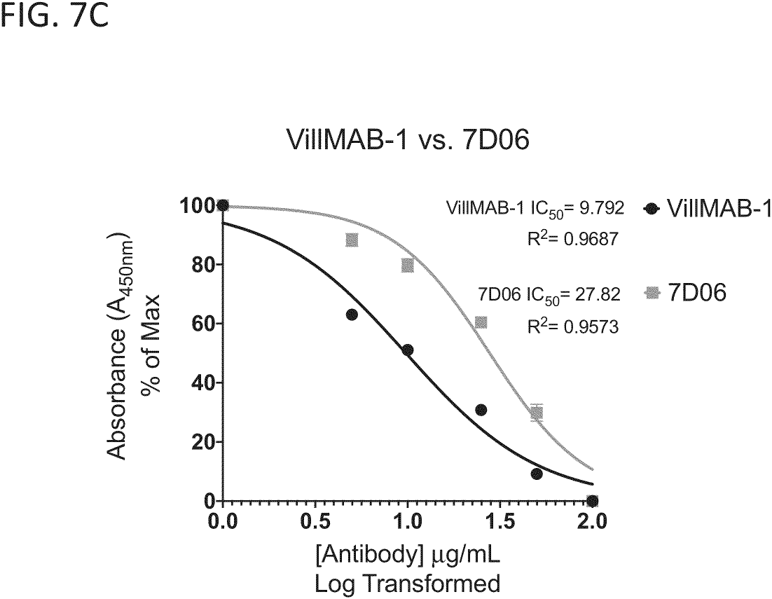

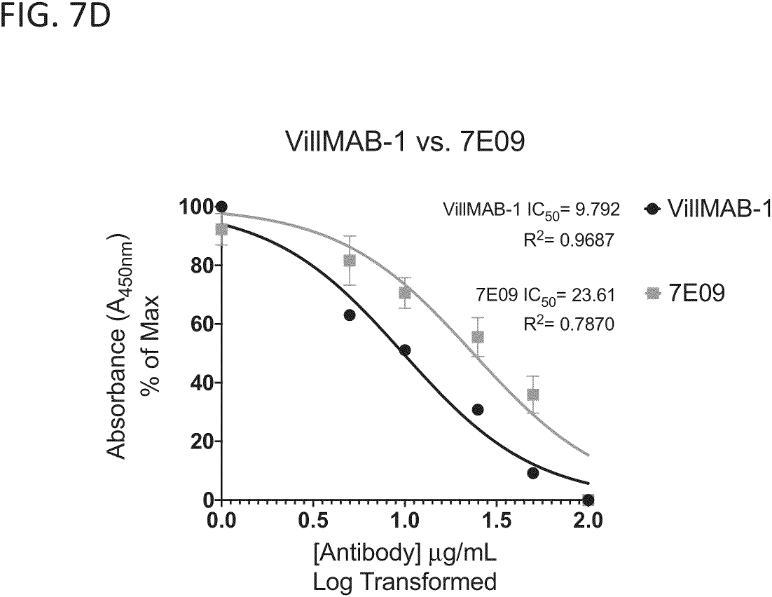

The 6 prioritized library-derived lead clones were analyzed for concentration-dependent binding to human and rhesus CD122 at the cell surface via flow cytometry (FIG. 6). Each of clones 06F 11 (FIG. 6A), 07C07 (FIG. 6B), 07D06 (FIG. 6C), 07E09 (FIG. 6D), 07D07 (FIG. 6E) and 06D 12 (FIG. 6F) exhibited CD122-specific binding profiles with highly similar binding curves to those observed for VillMab-1, while the isotype control IgG1 showed no binding to any cell type (FIG. 6G).

### Lead IgG Analyses in CD122-IL-15 Blockade Assay

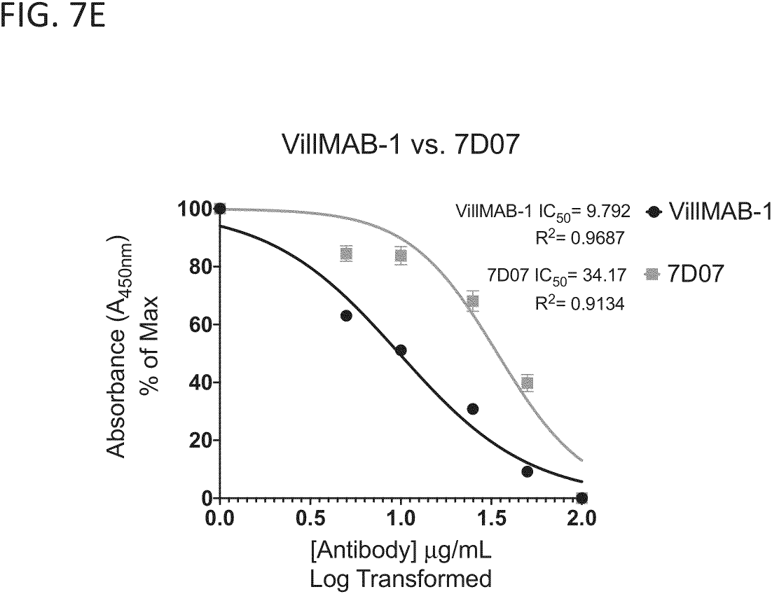

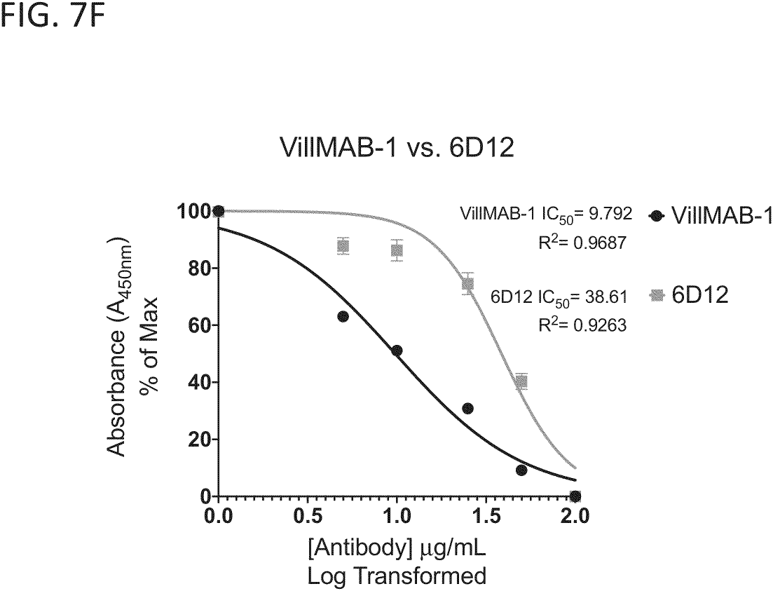

In a M07e cell-based CD122/IL-15 blockade reporter assay, clones 06F11 (FIG. 7A), 07C07 (FIG. 7B), 07D06 (FIG. 7C), 07E09 (FIG. 7D), 07D07 (FIG. 7E) and 06D12 (FIG. 7F) exhibited concentration-dependent antagonism of CD122. The IC50 for VillMAB-1 was 9.792 µg/mL. The IC50 for 6F11 and 7C07 were 14.8 µg/mL and 20.8 µg/mL respectively and were the lowest of the lead clones. The IC50 for 06D12, 07D06, 07D07 and 07E09 were 38.610 µg/mL, 27.820 µg/mL, 34.170 µg/mL and 23.610 µg/mL respectively. This analysis highlighted 06F11 and 07C07 as ideal candidates for further evaluation.

### Lead Fab Analyses in CD122 Binding BIACORE® for 1:1 Binding Affinity

To characterize true 1: 1 affinity values for clones 06F 11 and 07C07, plus a variant of 06F 11 which corrected a mutation in FW1 (06F11-V), these and the positive control Villmab-1 clones were clone, expressed and purified in human Fab format (i.e. monovalent and lacking both hinge and Fc regions). Fully-purified Fab proteins were examined for binding to both human CD122 (Table 10) and rhesus CD122 (Table 11). These analyses showed that clones FAB06F11-V, FAB06F11 and FAB07C07 exhibited moderately reduced overall KD values for both human and rhesus CD122, but, importantly, also exhibited both increased on (ka) and off (kd) rates, in comparison to VillFab-1.

### Lead IgG Analyses in hIL-15 NSG Mouse Model

A humanized hIL-15 NSG mouse model was used to compare the abilities of VillMAB-1, 7C07 and 6F11-v (all in IgG1-3M effector null format) to inhibit the CD122/IL15 signalling-supported engraftment of human NK and CD8 T cells in vivo. After establishing full engraftment with cells, mice were treated with vehicle, low dose (1 mg/kg) or high dose (10 mg/kg) antibody.

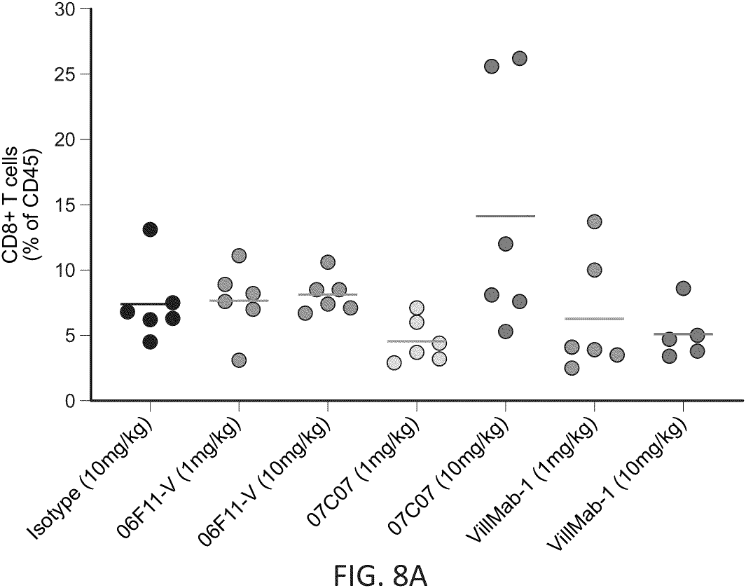

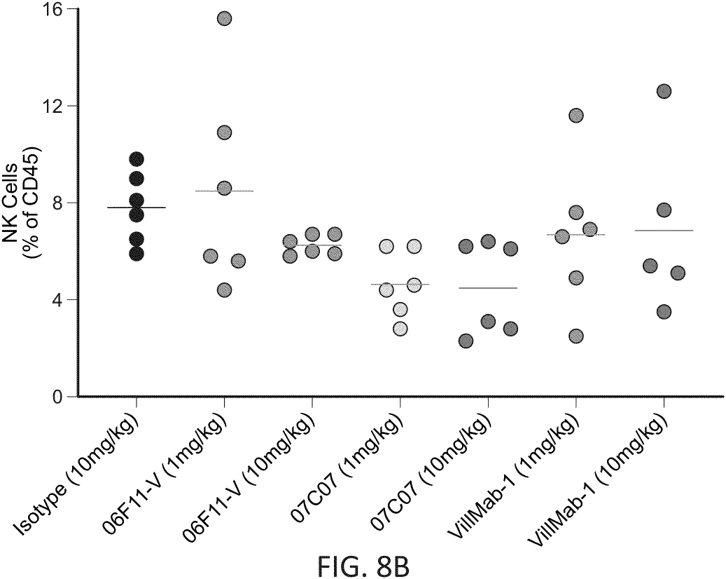

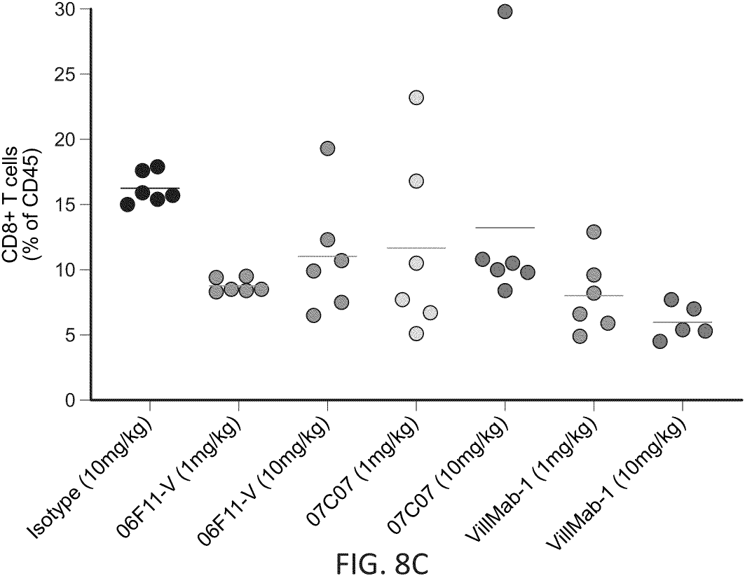

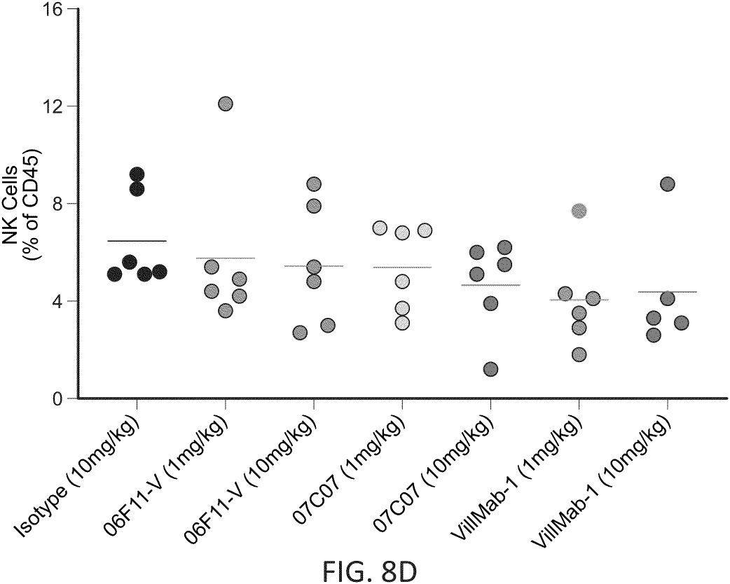

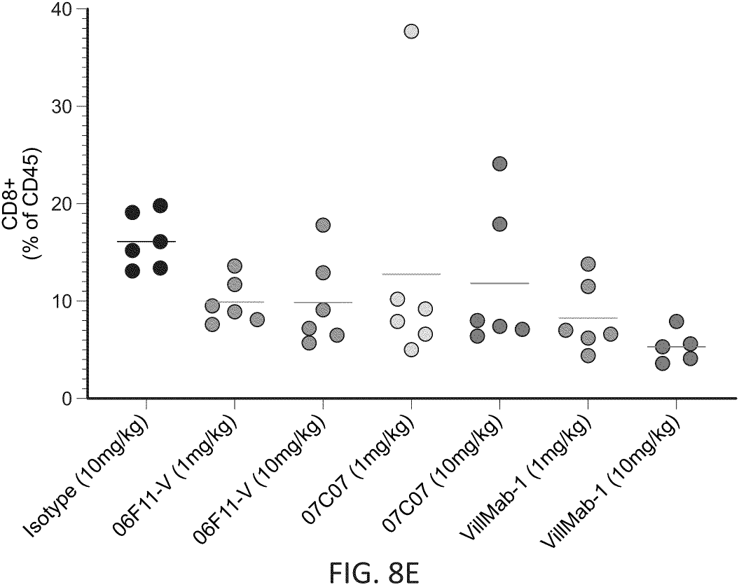

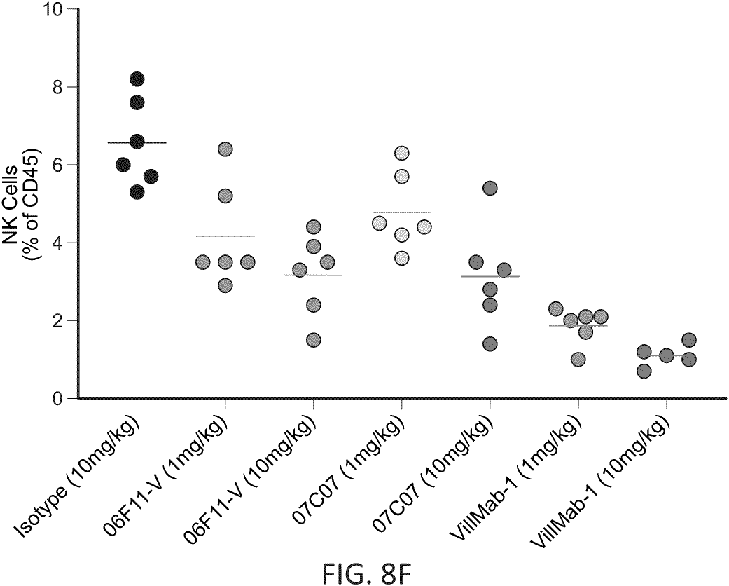

Prior to antibody treatment, numbers of human CD8+ T cells (FIG. 8A) and NK (FIG. 8B) cells in the blood were comparable between groups of mice. After 1 week of treatment, only mice treated with 10 mg/kg VillMAB-1 exhibited a statistically significant decrease in CD8+ T cell number compared to mice treated with isotype (FIG. 8C). A decrease in CD8+ T cells across all antibody treated groups was observed when compared to mice treated with isotype. Numbers of NK cells in the blood were comparable across all groups (FIG. 8D).

After 3 weeks of treatment, numbers of CD8+ T cells were decreased in the blood of all antibody treated groups compared to isotype treated mice (FIG. 8E). These changes were not statistically significant. Numbers of NK cells were decreased in all groups of antibody-treated mice and this decrease was statistically significant in every group, except mice treated with lmg/kg 07C07 (FIG. 8F). High and low dose VillMAB-1 treatment reduced NK cell numbers more than 06F11-V or 07C07 treatment at either dose.

Importantly, these findings confirmed that the chronic blockade of CD122-IL15 signaling by clones Villmab-1, 06F11-V or 07C07, in the absence of ADCC or ADCP effector functions, has the capacity to drive the depletion of both NK and CD8+ t cell populations.

### Antibody Binding Specificity Analyses for Clones 06F11-V and 07C07

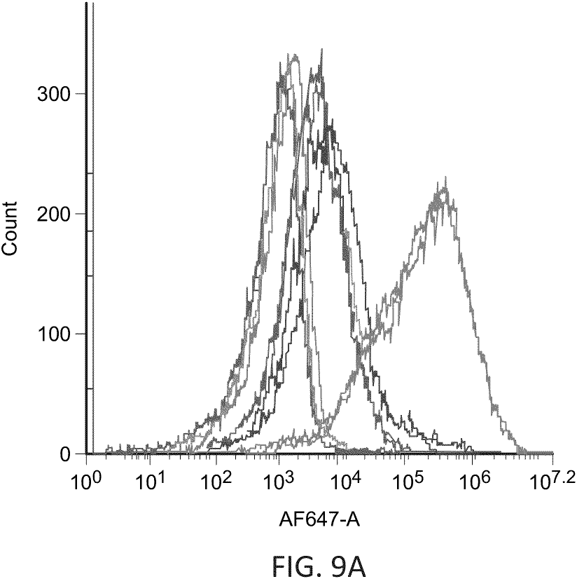

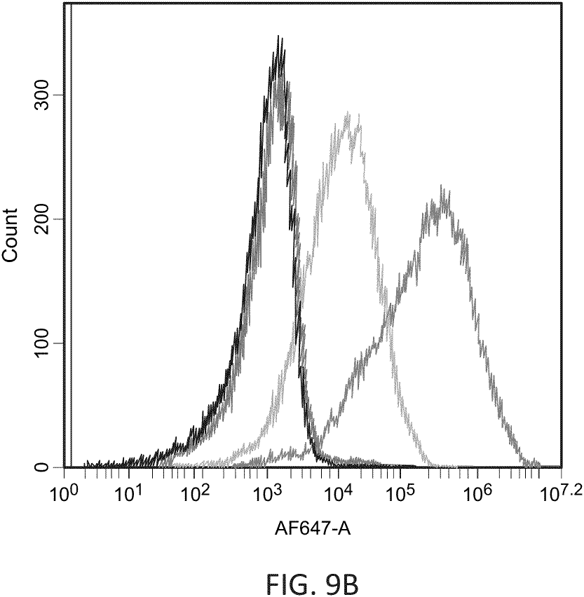

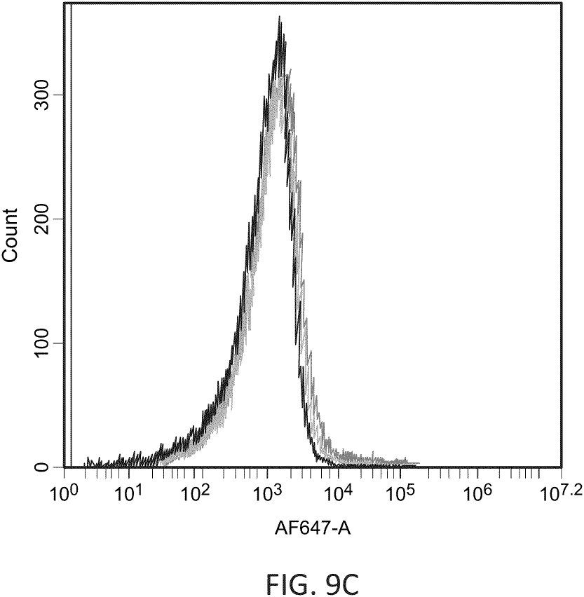

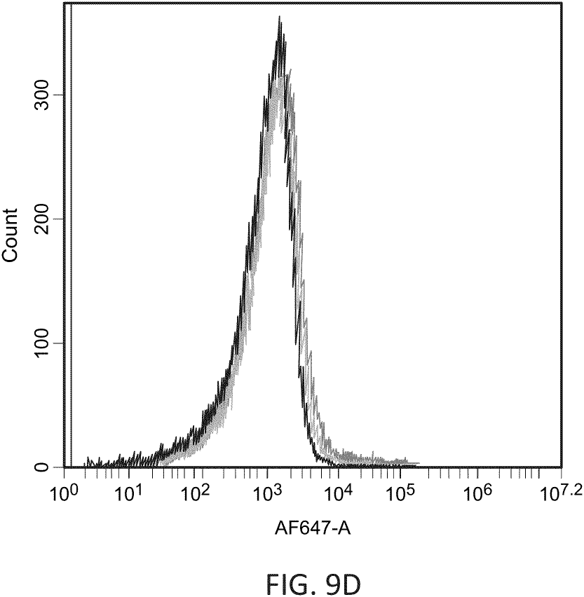

As clones 06F11-V and 07C07 were shown above to exhibit high levels of humanization in both framework regions and CDRs of the v-domains, plus effective blockade of CD122 in vitro and in vivo, they were used to re-screen for specificity analyses on the Retrogenix proteomics platform. Unexpectedly, this analysis demonstrated that the BCAM binding observed for Villmab-1 had been fully ablated and was not observed for either of 06F 11-V and 07C07 IgGs. In addition, however, two new interactions were observed that were not observed for Villmab-1 (FIG. 9). In flow cytometry analyses of binding to cells transfected with plasmids driving the expression of targets and controls, in addition to CD122 binding, clone 06F11-V was found to bind to both neudesin (a neurotropin) and CILP2 (a cartilage structural protein), while 07C07 was found to bind to CILP2 alone (FIG. 9A, FIG. 9B). In contrast, “secondary antibody only” (FIG. 9C) and Rituximab primary antibody (FIG. 9D) control experiments demonstrated either no binding over background, or binding only to CD20-transfected cells, respectively. These findings confirmed that the off-target binding observed for 06F11-V and 07C07 IgGs was genuine and specific.

### Optimization of Clone 06F11-V

Clone 06F 11-V was chosen for further optimization to maximize beneficial properties and to minimize off-target binding. This optimization was performed by experimental analysis of combinations of mutations back to murine sequences in CDRsl, and/or 2, and/or 3 of both the heavy and light chain sequences of 06F-11V. This process led to the creation of 18 new clones, each carrying one of 6 VH sequences combined with one of 3 VL sequences (Table 12). These 18 novel variants, Villmab-1 and 06F11V were cloned in human monovalent Fab fragment format, expressed in CHO cells and purified to monomeric state by protein A column, followed by SEC.

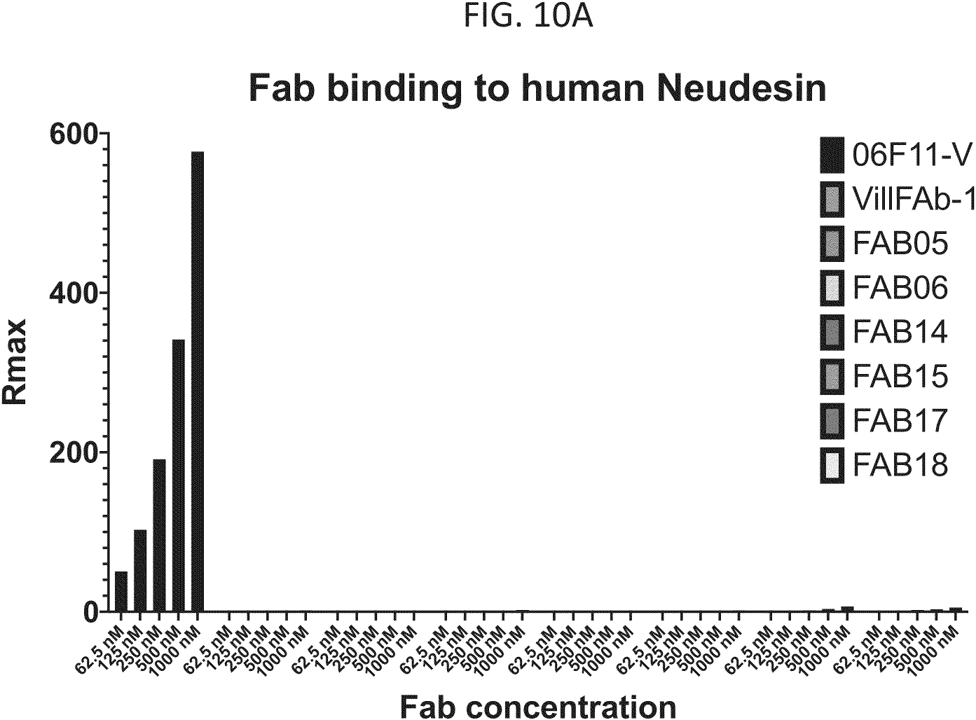

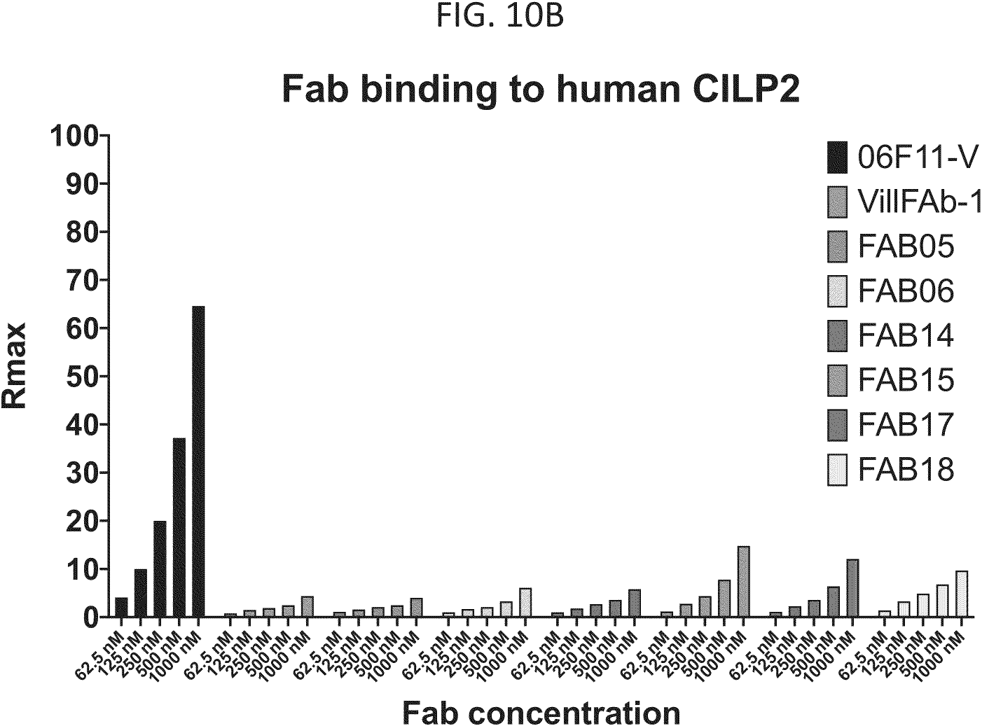

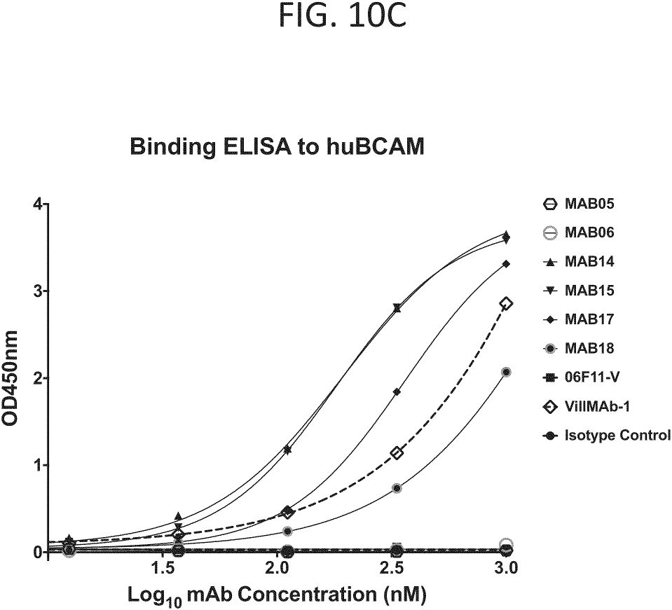

Purified Fabs were then examined for binding affinity to human and rhesus CD122 by BIACORE® (Table 13). This analysis showed that a series of clones exhibited improved affinity for CD122 over 06F11-V and even over VillMab-1. From this cohort, clones MAB05, MAB06, MAB14, MAB15, MAB17 and MAB18 were prioritized for further analysis and were further expressed and purified in human IgG1-3M format for potency and specificity analyses. When the Fab versions of all prioritized clones were tested in BIACORE® binding on human neudesin and CILP2 proteins (FIG. 10A, FIG. 10B), it was found that only clone 06F 11-V exhibited measurable binding to either protein, indicating that the novel lead clones had ablated these two off-target binding risks. In ELISA analyses of IgGs for all clones on human BCAM protein, all clones had retained off-target binding other than MAB05 and MAB06, which exhibited full specificity for CD122 (FIG. 10C, Table 14).

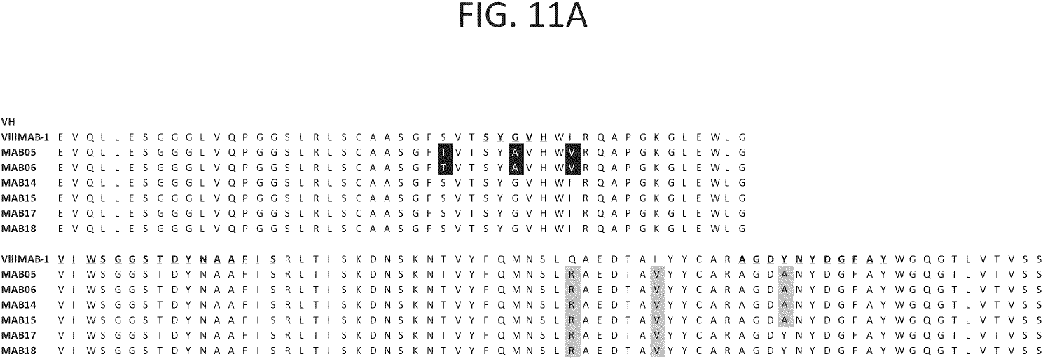

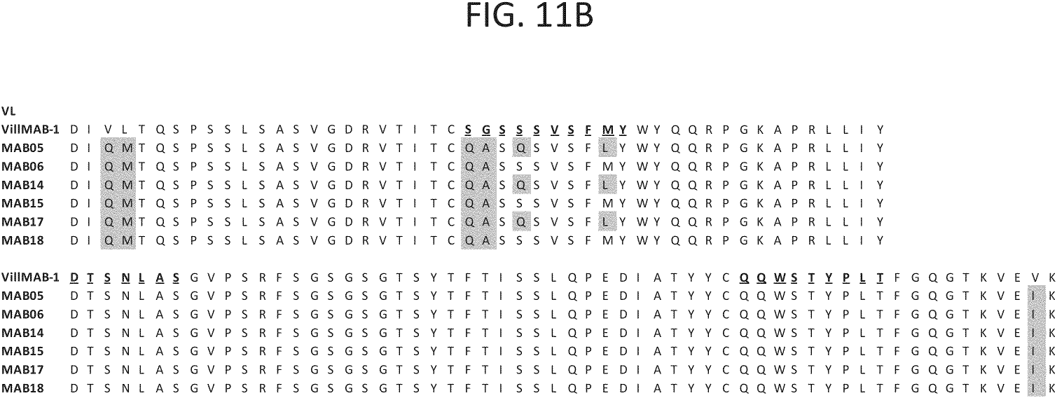

To examine how it is possible that such closely-related antibody sequences should have such radical differences in specificity profile, we performed sequence alignments of both the VH and VL domains for VillMab-1, versus clones 05, 06, 14, 15, 17 and 18 (FIGS. 11A, 11B). Remarkably, the only changes away from the VillMab-1 sequence that were unique to the (fully CD122-specific) clones MAB05 and MAB06 were 3 (highly homologous) mutations found in, and proximal to, the CDR1 of the VH domain. This finding illustrates the unpredictable nature of antibody binding promiscuity.

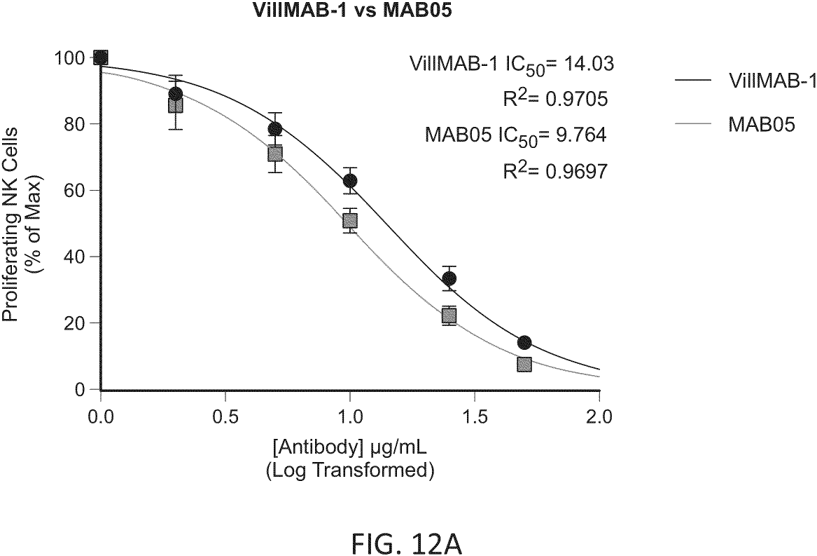

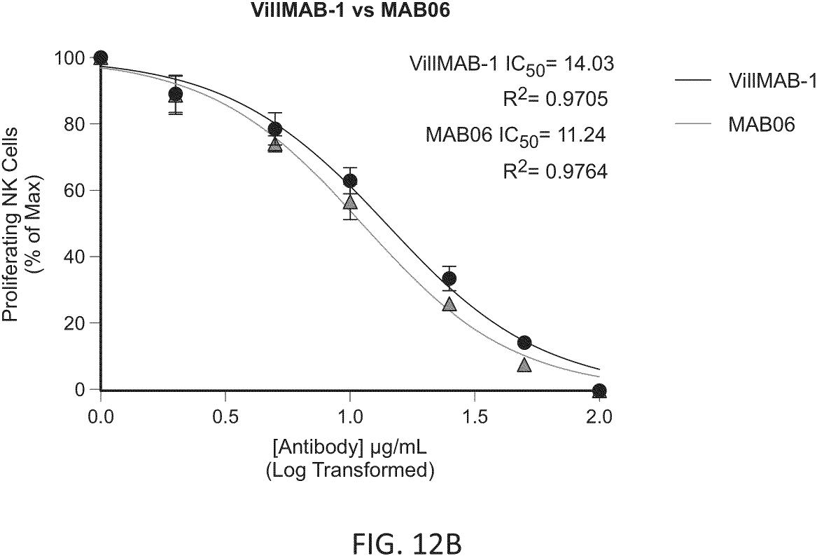

Confirmation of biological potency in clones MAB05, MAB06, MAB 14, MAB 15, MAB17 and MAB18 was ascertained in the IL-15 stimulated M07e assay (Table 14). This analysis demonstrated that not only is the affinity of clones MAB05 and MAB06 improved over VillMab-1, but their potency in blocking IL-15 signalling is also improved by approximately two-fold (Table 14). Final functionally relevant potency of clones MAB05 AND MAB06 was then ascertained in an assay measuring the proliferation of human primary NK cells under IL-15 stimulation (FIG. 12A, FIG. 12B). This assay recapitulated the findings in the M07e assay, demonstrating that MAB05 and MAB06 are indeed improved over VillMab-1.

### IgG1-3M BIACORE® Analyses in Binding to Human and Murine Fc Receptors

To characterize the affinity of clone 06F 11-V IgG1-3M and positive control IgGs, fully purified proteins were examined for binding to both human and murine FcγRs and FcRn (Table 17). These analyses showed that while all positive controls exhibited their expected strong interactions with both human and murine receptors, clone 06F11-V IgG1-3M exhibited very low or no measurable affinity for either human or murine FcγRs and FcRn at pH 7.4. Importantly, however, 06F11-V IgG1-3M retained full affinity for human and murine FcRn at pH 6.0. These findings confirmed the above in vivo observations in the NSG/IL-15 mouse model, that blockade of CD122 signaling in the absence of IgG effector function is sufficient to deplete CD 122+ cells.

### Example 2. Anti-CD122 Therapeutic Antibody Function in Human Skin T Cell Crawl Out Assay

A human skin biopsy culture assay was used to assess the ability of MAB05 and MAB06 to inhibit CD122/IL-15 signalling in skin-resident T cells.

Human skin biopsies (4 mm diameter × 2 mm thick) were harvested from surgical specimens (panniculectomy) using 4 mm Integra disposable biopsy punches (Integra). Skin biopsies were incubated in Antibiotic-Antimycotic (Gibco) diluted in PBS for 30 minutes at 4° C. and then rinsed 3x with PBS. Three skin biopsies were placed in 1 well of a 24-well plate (Corning) and briefly allowed to dry to promote adherence of the biopsies to the surface of the well. Biopsies were then cultured in 2 mL of Iscove’s modified medium (Sigma) with 20% heat-inactivated fetal bovine serum, penicillin and streptomycin (Corning), and 3.5 µL/L β-mercaptoethanol (Sigma) and incubated at 37° C. for 21 days. Cultures were fed three times per week by aspirating 1 mL of media from each well and adding back 1 mL of fresh media. For cultures treated with IL-15 or anti-CD122 antibody, 20 ng/mL of recombinant human IL15 (rhIL15) (R&D) and anti-CD122 antibody (MAB05 or MAB06) were added from the initiation of culture until collection of T cells at 21 days. After 21 days, culture media was harvested from wells and spun down at 330×g in SmL polystyrene round-bottom tubes for 10 minutes. The supernatant was aspirated, and the remaining T cells were washed, stained with anti-human CD3, CD4 and CD8 (1:20 dilution, Biolegend), and quantified with a BD LSR II flow cytometer (BD Biosciences) and FlowJo (Tree Star Inc.).

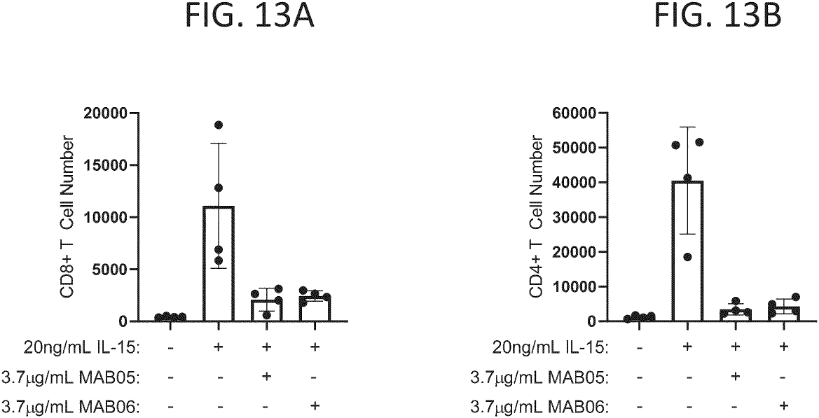

In this assay, MAB05 and MAB06 inhibit the IL15-induced accumulation of CD8+ T cells migrating from skin biopsies. More CD8+ T cells accumulate when biopsies are cultured with IL-15 than when biopsies are cultured without IL-15; 11,101 ± 6011 vs. 438.3 ± 66.05 (mean ± SD) (FIG. 13A). When biopsies are cultured with IL-15 and MAB05, the number of CD8+ T cells that accumulate is reduced compared to cultures with IL-15 alone; 2096 ± 1100 vs. 11,101 ± 6011 (mean ± SD) (FIG. 13A). Similarly, when biopsies are cultured with IL-15 and MAB06, fewer CD8+ T cells accumulate compared to cultures with IL-15 alone; 2436 ± 501.6 vs. 11,101 ± 6011 (mean ± SD) (FIG. 13A).

MAB05 and MAB06 also inhibit the IL15-induced accumulation of CD4+ T cells migrating from skin biopsies. More CD4+ T cells accumulate when biopsies are cultured with IL-15 than when biopsies are cultured without IL-15; 40,523 ± 15,391 vs. 1261 ± 473.6 (mean ± SD) (FIG. 13B). When biopsies are cultured with IL-15 and MAB05, the number of CD4+ T cells that accumulate is reduced compared to cultures with IL-15 alone; 3471 ± 1627 vs. 40,523 ± 15,391 (mean ± SD) (FIG. 13B). Similarly, when biopsies are cultured with IL-15 and MAB06, fewer CD4+ T cells accumulate compared to cultures with IL-15 alone; 4308 ± 2111 vs. 40,523 ± 15,391 (mean ± SD) (FIG. 13B).

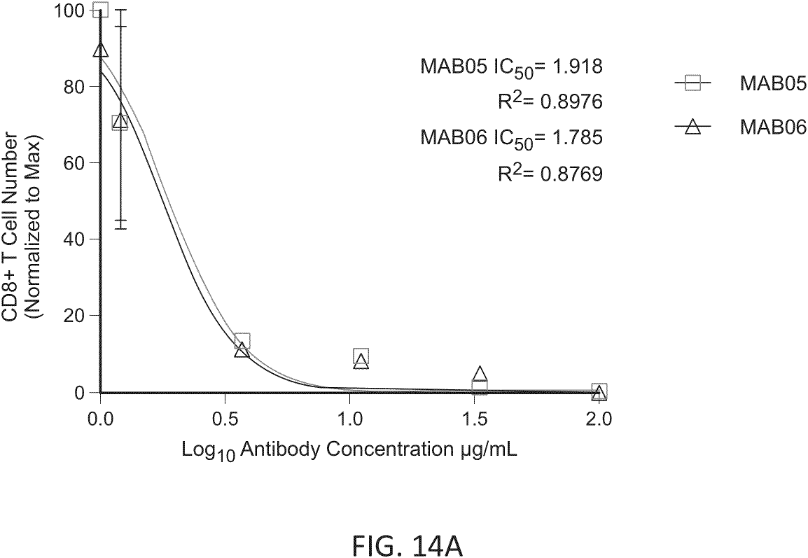

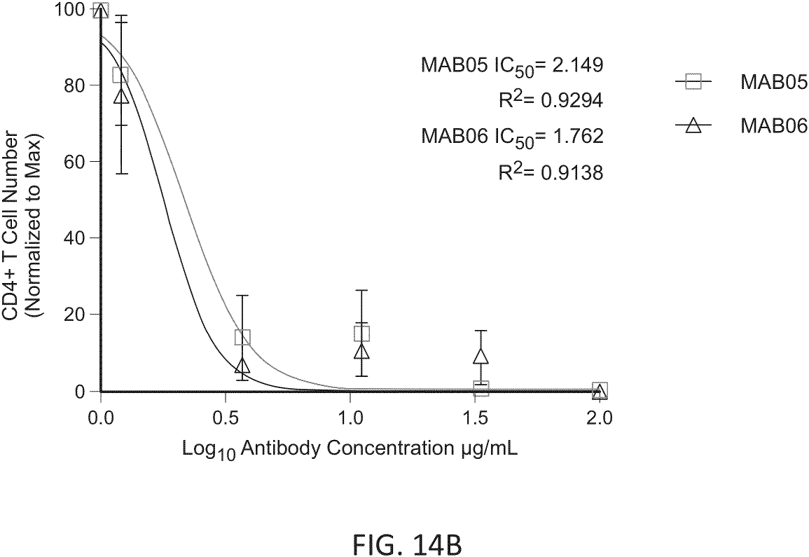

In skin biopsy culture assays, MAB05 exhibited a concentration-dependent antagonism of IL-15-induced skin-resident CD8+ T cell accumulation with an IC50 of 1.9 µg/mL (FIG. 14A). MAB06 exhibited a comparable concentration-dependent antagonism of CD8+ T cell accumulation with an IC50 of 1.8 µg/mL (FIG. 14A). In addition, MAB05 and MAB06 exhibited comparable concentration-dependent antagonism of IL-15-induced CD4+ T cell accumulation with an IC50 of 2.1 µg/mL and 1.8 µg/mL respectively (FIG. 14B).

### Example 3. Pharmacokinetic/Pharmacodynamic (PK/PD) Studies of Anti-CD122 Therapeutic Antibodies in Cynomolgus Monkeys

Cynomolgus monkeys were administered a single intravenous infusion of anti-CD122 antibody (MAB05 or MAB06) at a dose level ranging from 1 to 20 mg/kg. Blood samples were collected at pre-dose and at various timepoints ranging from 1 hour post-dose to day 16 post-dose. Pharmacokinetic parameters were determined following quantification of anti-CD122 antibody plasma concentrations by an ELISA method. Blood samples were also analyzed for CD122 receptor occupancy on NK cells and quantification of total T cells, helper T cells, cytotoxic T cells and NK cells, using flow cytometry methods.

Following single intravenous administration at dose levels ranging from 1 mg/kg to 20 mg/kg, MAB05 and MAB06 showed no clinical abnormalities and exhibited linear pharmacokinetics in cynomolgus monkeys. A single dose of MAB05 and MAB06 at 1 mg/kg was sufficient to maintain >90% CD122 receptor occupancy on NK cells throughout the sampling period (16 days post-dose). Administration of a single dose of MAB05 and MAB06 at 1 mg/kg, 5 mg/kg, or 20 mg/kg induced a decrease in circulating NK cell numbers. Circulating NK cells reached a nadir at approximately 7 days post-dose and showed a steady recovery following the 1 mg/kg or 5 mg/kg dose throughout the remaining sampling period (16 days post-dose). The modulation of circulating NK cell numbers is believed to be a marker of functional activity and is critical to demonstrate that the anti-CD122 antibody is effective in vivo (See Waldmann et al. (2020) J Exp Med217:e20191062), and therefore could be advantageously used to define optimal dosing in patients. No effect was observed on helper T cells or cytotoxic T cells.

### Example 4. Pre-Formulation Development and Stability Studies of Anti-CD122 Therapeutic Antibodies

Anti-CD122 antibodies were evaluated for manufacturing developability using a panel of stability studies. Ten formulations of anti-CD122 antibodies were assessed from a combination of buffer and excipients for stability and aggregation potential at a concentration of 5 mg/mL. The antibodies were assessed under conditions of low pH stress, heat stress, freeze-thaw conditions and forced oxidation. Antibodies were also evaluated for self-association and viscosity at concentrations >100 mg/mL.

MAB05 and MAB06 demonstrated high thermal stability, high tolerance to low pH (pH 3.0), low aggregation potential, low oxidation and deamidation sensitivity, and excellent freeze/thaw stability in a buffer with a composition of 25 mM L-histidine, 9% (w/v) sucrose, 0.02% (w/v) polysorbate 80, pH 6.0. MAB05 and MAB06 were solubilized in this same buffer solution at concentrations ranging from 90 to 120 mg/mL. The viscosity of MAB05 and MAB06 at a concentration of 115 mg/mL was low (approximately 4-6 centipoise), indicating feasibility for achieving a clinical formulation for subcutaneous delivery of the anti-CD122 antibody.

TABLE 1

Target biology input parameters for pharmacological modelling of anti-CD122 IgG

| | | |
|---|---|---|
| Parameter | Systemic Compartment Value | SOA Compartment Value |
| Receptor-Ligand (KD) | N/A | 1 nM (Bouchaud et al. J. Mol. Biol. (2008) 382, 1-12) |
| CD122 Expression | 0.24 nM | 0.32 nM |
| CD122 turnover | 110 min (Hemar et al. Eur. J. Immunol. 1994. 24: 1951-1955, Smith et al. PNAS (1985) 82:864-868, Mortier et al. J Biol Chem. 2006; 281(3):1612-9) | |
| Ligand (IL-15) Expression | N/A | 0.14 nM |
| Ligand Turnover | 24 hours (Dubois et al. Immunity, 17: 537-547, 2002) | |
| Volume | 5.2 L (distribution volume) | 0.164 L (interstitial volume of skin, Radtke et al. |
| | | Dermatology 2010; 220: 194-200) |

TABLE 2

Drug input parameters for pharmacological modelling of anti-CD122 IgG

| | |
|---|---|
| Drug Parameter | Parameter values |
| Dosing Schedule | Q4W (IV) or Q1W (SC) |
| Dose | 700 mg max IV |
| | 100 mg max SC |
| Administration route | IV or SC |
| Affinity for CD122 (KD) | > 15 nM |
| T ½ | 16d (typical IgG half life) |
| SC absorption T ½ | 2.5 days (typical antibody absorption half-life) |
| Drug MW (kDa) | 150 kDa |
| T dist | 6 hours (Typical IgG value) |
| P dist (distribution systemic to skin) | 0.23 (Based on measured antibody distribution to skin (Dragatin et al. Experimental Dermatology, 2016, 25, 151-164; Jadhav et al. Journal of Pharmaceutical Sciences 106 (2017) 2853-2859, Kratochwil et al (2018) PLoS ONE 13(10): e0205435, Shah and Betts (2013) mAbs 5:2, 297-305)) |

TABLE 3

IV Q4W dosing: effect of drug affinity for CD122 on dosing requirements (Minimum Dose Required to Meet Inhibition Criteria at Different Drug Affinities)

| Trough RO In Skin | Dose (InM KD) | Dose (10 nM KD) | Dose (20 nM KD) |
|---|---|---|---|
| 85% | 144 mg | 396 mg | 677 mg |
| 90% | 163 mg | 563 mg | 1010 mg |
| 95% | 219 mg | 1070 mg | 2020 mg |
| 99% | 661 mg | 5100 mg | 10000 mg |

TABLE 4

SC Q1W dosing: SC dosing requirements with 10 nM KD (Projected Dose to Achieve RO in the skin, assuming 100 mg is max viable SC dose)

| Trough RO In Skin | Q1W | Q2W | Q3W |
|---|---|---|---|
| 85% | 56 mg | 121 mg | 214 mg |
| 90% | 81 mg | 563 mg | 1010 mg |
| 95% | 155 mg | 1070 mg | 2020 mg |
| 99% | 749 mg | 5100 mg | 10000 mg |

TABLE 5

IV Q4W: Dose to target RO with no systemic burden (10 nM IgG KD)

| Trough RO In Skin | Dose (with systemic CD122) | Dose (with no systemic CD122) |
|---|---|---|
| 85% | 396 mg | 121 mg |
| 90% | 565 mg | 563 mg |
| 95% | 1070 mg | 1070 mg |
| 99% | 5100 mg | 5100 mg |

TABLE 6

Amino acid sequence of humanized anti-CD122 variable regions

| | |
|---|---|
| ΜΙΚβ1 VH | EVQLLESGGGLVQPGGSLRLSCAASGFSVTSYGVH WIRQAPGKGLEWLGV IWSGGSTDYNAAFIS RLTISKDNSKNTVYFQMNSLQAEDTAIYYCARAGDYNYDGFAYWGQGTLVTVSS (SEQ ID NO: 22) |
| ΜΙΚβ1 VL | DIVLTQSPSSLSASVGDRVTITCSGSSSVSFMY WYQQRPGKAPRLLIYDTSNLAS GVPSRFSGSGSGTSYTFTISSLQPEDIATYYCQQWSTYPLTFGQGTKVEVK (SEQ ID NO: 28) |

Bold = CDR
Underlined = differing from human germlines IGHV3-23/JH4 (VH) and IGKV1-33/J4 (VL)

TABLE 7

Variable region sequences of top 15 unique, library-derived CD122 antagonistic IgGs

| | | | | | | |
|---|---|---|---|---|---|---|
| Clone | HCDR1 | HCDR2 | HCDR3 | LCDR1 | LCDR2 | LCDR3 |
| 07C07 | SYGVH (SEQ ID NO: 24) | AIWSGGSTDYAASVKG (SEQ ID NO: 58) | AGDYNYDGFAY (SEQ ID NO: 27) | QASQDISFMY (SEQ ID NO: 112) | DASNLAT (SEQ ID NO: 149) | QQWDNYPLT (SEQ ID NO: 153) |
| 07D06 | SYAMH (SEQ ID NO: 56) | AIWSGGSTDYADAVKG (SEQ ID NO: 59) | AGDYEYDGFAY (SEQ ID NO: 92) | QASQSVSHLY (SEQ ID NO: 113) | DTSNLAT (SEQ ID NO: 150) | QQWSTYPLT (SEQ ID NO: 15) |
| 07E09 | SYGMH (SEQ ID NO: 57) | VIWSGGSTDYADSVKG (SEQ ID NO: 60) | AGDYNYDGFAY (SEQ ID NO: 27) | QASQSVSYMY (SEQ ID NO: 114) | DASNLAT (SEQ ID NO: 149) | QQWDTYPLT (SEQ ID NO: 154) |
| 07F11 | SYGVH (SEQ ID NO: 24) | AIWSGGSTDYADAVKG (SEQ ID NO: 59) | AGDKNYDGFAY (SEQ ID NO: 93) | QASSSISFMY (SEQ ID NO: 115) | DASNLAT (SEQ ID NO: 149) | QQWDTYPLT (SEQ ID NO: 154) |
| 07D07 | SYGVH (SEQ ID NO: 24) | AIWSGGSTDYADAVKG (SEQ ID NO: 59) | AGDKNYDGFAY (SEQ ID NO: 93) | QASSSISYMY (SEQ ID NO: 116) | DASNLAT (SEQ ID NO: 149) | QQWDNYPLT (SEQ ID NO: 153) |
| 07C02 | SYGVH (SEQ ID NO: 24) | AIWSGGSTDYADAVKG (SEQ ID NO: 59) | AGDKNYDGFAY (SEQ ID NO: 93) | QASSSVSHMY (SEQ ID NO: 117) | DTSNLAT (SEQ ID NO: 150) | QQWSTYPLT (SEQ ID NO: 15) |
| 07C12 | SYGVH (SEQ ID NO: 24) | AIWSGGSTDYADAVKG (SEQ ID NO: 59) | AGDKNYDGFAY (SEQ ID NO: 93) | QASQSISFLY (SEQ ID NO: 118) | DASNLAT (SEQ ID NO: 149) | QQWSTYPLT (SEQ ID NO: 15) |
| 07D08 | SYGVH (SEQ ID NO: 24) | AIWSGGSTDYADAVKG (SEQ ID NO: 59) | AGDKNYDGFAY (SEQ ID NO: 93) | QASQSISFLY (SEQ ID NO: 118) | DTSNLAT (SEQ ID NO: 150) | QQWSNYPLT (SEQ ID NO: 155) |
| 07D11 | SYGVH (SEQ ID NO: 24) | AIWSGGSTDYADAVKG (SEQ ID NO: 59) | AGDKNYDGFAY (SEQ ID NO: 93) | QASQSISHLY (SEQ ID NO: 119) | DTSNLET (SEQ ID NO: 151) | QQWDNYPLT (SEQ ID NO: 153) |
| 07C08 | SYGVH (SEQ ID NO: 24) | AIWSGGSTDYADAVKG (SEQ ID NO: 59) | AGDKNYDGFAY (SEQ ID NO: 93) | QASQSISHMY (SEQ ID NO: 120) | DTSNLET (SEQ ID NO: 151) | QQWSTYPLT (SEQ ID NO: 15) |
| 07D12 | SYGVH (SEQ ID NO: 24) | AIWSGGSTDYADAVKG (SEQ ID NO: 59) | AGDKNYDGFAY (SEQ ID NO: 93) | QASSDISHLY (SEQ ID NO: 121) | DTSNLAT (SEQ ID NO: 150) | QQWDTYPLT (SEQ ID NO: 154) |
| 06D12 | SYGVH (SEQ ID NO: 24) | VIWSGGSTDYADAVKG (SEQ ID NO: 61) | AGDQNYDGFAY (SEQ ID NO: 94) | QASSSVSHLY (SEQ ID NO: 122) | DTSNLET (SEQ ID NO: 151) | QQWDTYPLT (SEQ ID NO: 154) |
| 07H10 | SYAMH (SEQ ID NO: 56) | VIWSGGSTDYNDAVKG (SEQ ID NO: 62) | AGDYEYDGFAY (SEQ ID NO: 92) | QASQSISFLY (SEQ ID NO: 118) | DTSNLAT (SEQ ID NO: 150) | QQWDNYPLT (SEQ ID NO: 153) |
| 07A02 | SYAVH (SEQ ID NO: 3) | AIWSGGSTDYAAAVKG (SEQ ID NO: 63) | AGDANYDGFAY (SEQ ID NO: 7) | QASQSISYMY (SEQ ID NO: 123) | DTSNLAT (SEQ ID NO: 150) | QQWDNYPLT (SEQ ID NO: 153) |
| 06F11 | SYAVH (SEQ ID NO: 3) | AIWSGGSTDYNAAVKG (SEQ ID NO: 66) | AGDANYDGFAY (SEQ ID NO: 7) | QASQSVSFLY (SEQ ID NO: 11) | DTSNLAT (SEQ ID NO: 150) | QQWDTYPLT (SEQ ID NO: 154) |

TABLE 8

Unique CDR sequences found in library-derived, CD122-binding antibodies.

| | | | | | |
|---|---|---|---|---|---|
| HCDR1 | HCDR2 | HCDR3 | LCDR1 | LCDR2 | LCDR3 |
| SYAMH (SEQ ID NO: 56) | AIWSGGSTDYAAAVKG (SEQ ID NO: 63) | AGDANYDGFAY (SEQ ID NO: 7) | QASQDISFMY (SEQ ID NO: 112) | DASNLAT (SEQ ID NO: 149) | QQWDNLPLT (SEQ ID NO: 156) |
| SYAVH (SEQ ID NO: 3) | AIWSGGSTDYAASVKG (SEQ ID NO: 58) | AGDENYDGFAY (SEQ ID NO: 95) | QASQDISHLY (SEQ ID NO: 124) | DASNLET (SEQ ID NO: 152) | QQWDNYPLT (SEQ ID NO: 153) |
| SYGMH (SEQ ID NO: 57) | AIWSGGSTDYAATVKG (SEQ ID NO: 64) | AGDHNYDGFAY (SEQ ID NO: 96) | QASQDISYLY (SEQ ID NO: 125) | DTSNLAT (SEQ ID NO: 150) | QQWDTLPLT (SEQ ID NO: 157) |
| SYGVH (SEQ ID NO: 24) | AIWSGGSTDYADAVKG (SEQ ID NO: 59) | AGDKNYDGFAY (SEQ ID NO: 93) | QASQDISYMY (SEQ ID NO: 126) | DTSNLET (SEQ ID NO: 151) | QQWDTYPLT (SEQ ID NO: 154) |
| | AIWSGGSTDYADSVKG (SEQ ID NO: 65) | AGDMNYDGFAY (SEQ ID NO: 97) | QASQDVSFLY (SEQ ID NO: 127) | | QQWSNLPLT (SEQ ID NO: 158) |
| | AIWSGGSTDYNAAVKG (SEQ ID NO: 66) | AGDNNYDGFAY (SEQ ID NO: 98) | QASQDVSHLN (SEQ ID NO: 128) | | QQWSNYPLT (SEQ ID NO: 155) |
| | AIWSGGSTDYNASVKG (SEQ ID NO: 67) | AGDNNYDGFEY (SEQ ID NO: 99) | QASQDVSHLY (SEQ ID NO: 129) | | QQWSTLPLT (SEQ ID NO: 159) |
| | AIWSGGSTDYNDAVKG (SEQ ID NO: 68) | AGDQNYDGFAY (SEQ ID NO: 94) | QASQDVSHMY (SEQ ID NO: 130) | | QQWSTYPLT (SEQ ID NO: 15) |
| | AIWSGGSTDYNDSVKG (SEQ ID NO: 69) | AGDYDYDGFAY (SEQ ID NO: 100) | QASQDVSYLY (SEQ ID NO: 131) | | |
| | AIWSGGSTQYNAAVKG (SEQ ID NO: 70) | AGDYEYDGFAY (SEQ ID NO: 92) | QASQDVSYMY (SEQ ID NO: 132) | | |
| | AIWSGGSTYYADAVKG (SEQ ID NO: 71) | AGDYNLDGFAY (SEQ ID NO: 101) | QASQSISFLY (SEQ ID NO: 118) | | |
| | AIWSGGSTYYNAAVKG (SEQ ID NO: 72) | AGDYNWDGFAY (SEQ ID NO: 102) | QASQSISFMY (SEQ ID NO: 133) | | |
| | AIWSGGSTYYNASVKG (SEQ ID NO: 73) | AGDYNYDGFAI (SEQ ID NO: 103) | QASQSISHLY (SEQ ID NO: 119) | | |
| | AIWSGGSTYYNDAVKG (SEQ ID NO: 74) | AGDYNYDGFAM (SEQ ID NO: 104) | QASQSISHMY (SEQ ID NO: 120) | | |
| | AIYSGGSTDYAAAVKG (SEQ ID NO: 75) | AGDYNYDGFAN (SEQ ID NO: 105) | QASQSISYMY (SEQ ID NO: 123) | | |
| | AIYSGGSTDYADAVKG (SEQ ID NO: 76) | AGDYNYDGFAW (SEQ ID NO: 106) | QASQSVRHMY (SEQ ID NO: 134) | | |
| | AIYSGGSTDYNAAVKG (SEQ ID NO: 77) | AGDYNYDGFAY (SEQ ID NO: 27) | QASQSVSFLY (SEQ ID NO: 11) | | |
| | AIYSGGSTDYNDAVKG (SEQ ID NO: 78) | AGDYNYDGFRY (SEQ ID NO: 107) | QASQSVSFMY (SEQ ID NO: 135) | | |
| | AIYSGGSTYYAASVKG (SEQ ID NO: 79) | AGDYNYDGLAY (SEQ ID NO: 108) | QASQSVSHLY (SEQ ID NO: 113) | | |
| | AIYSGGSTYYADAVKG (SEQ ID NO: 80) | AGNYNYDGFAY (SEQ ID NO: 109) | QASQSVSHMY (SEQ ID NO: 136) | | |
| | AIYSGGSTYYNDAVKG (SEQ ID NO: 81) | AGPYNYDGFAY (SEQ ID NO: 110) | QASQSVSYLY (SEQ ID NO: 137) | | |
| | VIWSGGSTDYAAAVKG (SEQ ID NO: 82) | AGTYNYDGFAY (SEQ ID NO: 111) | QASQSVSYMY (SEQ ID NO: 114) | | |
| | VIWSGGSTDYAASVKG (SEQ ID NO: 83) | | QASSDISFMY (SEQ ID NO: 138) | | |
| | VIWSGGSTDYADAVKG (SEQ ID NO: 61) | | QASSDISHLY (SEQ ID NO: 121) | | |
| | VIWSGGSTDYADSVKG (SEQ ID NO: 60) | | QASSDISYMY (SEQ ID NO: 139) | | |
| | VIWSGGSTDYNAAVKG (SEQ ID NO: 84) | | QASSDVSFLY (SEQ ID NO: 140) | | |
| | VIWSGGSTDYNASVKG (SEQ ID NO: 85) | | QASSDVSFMY (SEQ ID NO: 141) | | |
| | VIWSGGSTDYNDAVKG (SEQ ID NO: 62) | | QASSDVSHLY (SEQ ID NO: 142) | | |
| | VIWSGGSTDYNDSVKG (SEQ ID NO: 86) | | QASSDVSHMY (SEQ ID NO: 143) | | |
| | VIWSGGSTYYNDAVKG (SEQ ID NO: 87) | | QASSDVSYMY (SEQ ID NO: 144) | | |
| | VIYSGGSTDYAASVKG (SEQ ID NO: 88) | | QASSSISFLY (SEQ ID NO: 145) | | |
| | VIYSGGSTDYADSVKG (SEQ ID NO: 89) | | QASSSISFMY (SEQ ID NO: 115) | | |
| | VIYSGGSTDYNDAVKG (SEQ ID NO: 90) | | QASSSISHLY (SEQ ID NO: 146) | | |
| | VIYSGGSTYYNAAVKG (SEQ ID NO: 91) | | QASSSISYLY (SEQ ID NO: 147) | | |
| | | | QASSSISYMY (SEQ ID NO: 116) | | |
| | | | QASSSVSHLY (SEQ ID NO: 122) | | |
| | | | QASSSVSHMY (SEQ ID NO: 117) | | |
| | | | QASSSVSYMY (SEQ ID NO: 148) | | |

TABLE 9

Designer variable domain sequences used in pairwise combination to create MAB01-MAB15 IgGls

| | |
|---|---|
| DVH1 | EVQLLESGGGLVQPGGSLRLSCAASGFSVSSYGMHWVRQAPGKGLEWLGA IWSGGSTDYADAVKGRFTISRDNSK NTLYLQMNSLRAEDTAVYYCARAGDKNYDGFAYWGQGTLVTVSS (SEQ ID NO: 41) |
| DVH2 | EVQLLESGGGLVQPGGSLRLSCAASGFSVSSYGMHWVRQAPGKGLEWLGV IWSGGSTDYADAVKGRFTISRDNSK NTLYLQMNSLRAEDTAVYYCARAGDKNYDGFAYWGQGTLVTVSS (SEQ ID NO: 42) |
| DVH3 | EVQLLESGGGLVQPGGSLRLSCAASGFSVSSYGMHWVRQAPGKGLEWLGA IWSGGSTDYADAVKGRFTISRDNSK NTLYLQMNSLRAEDTAVYYCARAGDHNYDGFAYWGQGTLVTVSS (SEQ ID NO: 43) |
| DVH4 | EVQLLESGGGLVQPGGSLRLSCAASGFSVSSYGMHWVRQAPGKGLEWLGV IWSGGSTDYADAVKGRFTISRDNSK NTLYLQMNSLRAEDTAVYYCARAGDHNYDGFAYWGQGTLVTVSS (SEQ ID NO: 44) |
| DVH5 | EVQLLESGGGLVQPGGSLRLSCAASGFSVSSYGMHWVRQAPGKGLEWLGA IWSGGSTDYNDAVKGRFTISRDNSK NTLYLQMNSLRAEDTAVYYCARAGDHNYDGFAYWGQGTLVTVSS (SEQ ID NO: 45) |
| DVL1 | DIQMTQSPSSLSASVGDRVTITCQASQSISYMYWYQQRPGKAPKLLIYDT SNLATGVPSRFSGSGSGTSYTFTIS SLQPEDIATYYCQQWDNYPLTFGGGTKVEIK (SEQ ID NO: 46) |
| DVL2 | DIQMTQSPSSLSASVGDRVTITCQASQDISYLYWYQQRPGKAPKLLIYDA SNLATGVPSRFSGSGSGTSYTFTIS SLQPEDIATYYCQQWDNYPLTFGGGTKVEIK (SEQ ID NO: 47) |
| DVL3 | DIQMTQSPSSLSASVGDRVTITCQASQDISYLYWYQQRPGKAPKLLIYDA SNLETGVPSRFSGSGSGTSYTFTIS SLQPEDIATYYCQQWDNYPLTFGGGTKVEIK (SEQ ID NO: 48) |

TABLE 10

BIACORE® affinity values for Fab binding to human CD122

| Analyte | Chi² (RU²) | ka (⅟Ms) | kd (⅟s) | KD (M) | KD (nM) |
|---|---|---|---|---|---|
| VillFab1 | 0.14 | 2.26E+05 | 3.72E-03 | 1.65E-08 | 16.46 |
| FAB06F11-V | 1.3 | 7.38E+06 | 1.30E-01 | 1.76E-08 | 17.64 |
| FAB06F11 | 0.36 | 6.38E+06 | 1.52E-01 | 2.39E-08 | 23.85 |
| FAB07C07 | 0.09 | 3.65E+06 | 1.55E-01 | 4.25E-08 | 42.49 |

TABLE 11

BIACORIE® affinity values for Fab binding to rhesus CD122

| Analyte | Chi² (RU²) | ka (⅟Ms) | kd (⅟s) | KD (M) | KD (nM) |
|---|---|---|---|---|---|
| VillFab1 | 0.09 | 2.33E+05 | 2.79E-03 | 1.20E-08 | 11.98 |
| FAB06F11-V | 2.4 | 5.00E+06 | 6.06E-02 | 1.21E-08 | 12.13 |
| FAB06F11 | 0.92 | 4.37E+06 | 6.50E-02 | 1.49E-08 | 14.88 |
| FAB07C07 | 0.56 | 3.31E+06 | 8.40E-02 | 2.54E-08 | 25.35 |

Table 12

Variable domains used in 06F11-V optimization
VH sequences
F11VH-1
EVQLLESGGGLVQPGGSLRLSCAASGFSVTSYGVHWIRQAPGKGLEWLGA IWSGGSTQYNAAVKGRFTISRDNSKNTLYLQMNSLRAEDTAVYYCARAGD ANYDGFAYWGQGTLVTVSS (SEQ ID NO: 49)

F11VH-2
EVQLLESGGGLVQPGGSLRLSCAASGFTVTSYAVHWVRQAPGKGLEWLGV IWSGGSTDYNAAFISRLTISKDNSKNTVYFQMNSLRAEDTAVYYCARAGD ANYDGFAYWGQGTLVTVSS (SEQ ID NO: 1)

F11VH-13
EVQLLESGGGLVQPGGSLRLSCAASGFSVTSYGVHWIRQAPGKGLEWLGA IWSGGSTQYNAAVKGRFTISRDNSKNTLYLQMNSLRAEDTAVYYCARAGD YNYDGFAYWGQGTLVTVSS (SEQ ID NO: 50)

F11VH-23
EVQLLESGGGLVQPGGSLRLSCAASGFTVTSYAVHWVRQAPGKGLEWLGV IWSGGSTDYNAAFISRLTISKDNSKNTVYFQMNSLRAEDTAVYYCARAGD YNYDGFAYWGQGTLVTVSS (SEQ ID NO: 51)

F11VH-12
EVQLLESGGGLVQPGGSLRLSCAASGFSVTSYGVHWIRQAPGKGLEWLGV IWSGGSTDYNAAFISRLTISKDNSKNTVYFQMNSLRAEDTAVYYCARAGD ANYDGFAYWGQGTLVTVSS (SEQ ID NO: 52)

F11VH-123
EVQLLESGGGLVQPGGSLRLSCAASGFSVTSYGVHWIRQAPGKGLEWLGV IWSGGSTDYNAAFISRLTISKDNSKNTVYFQMNSLRAEDTAVYYCARAGD YNYDGFAYWGQGTLVTVSS (SEQ ID NO: 53)

VK sequences
F11VK-12
DIQMTQSPSSLSASVGDRVTITCQASSSVSFMYWYQQRPGKAPRLLIYDT SNLASGVPSRFSGSGSGTSYTFTISSLQPEDIATYYCQQWDTYPLTFGGG TKVEIK (SEQ ID NO: 54)D

F11VK-23
DIQMTQSPSSLSASVGDRVTITCQASQSVSFLYWYQQRPGKAPRLLIYDT SNLASGVPSRFSGSGSGTSYTFTISSLQPEDIATYYCQQWSTYPLTFGQG TKVEIK (SEQ ID NO: 9)

F11VK-123
DIQMTQSPSSLSASVGDRVTITCQASSSVSFMYWYQQRPGKAPRLLIYDT SNLASGVPSRFSGSGSGTSYTFTISSLQPEDIATYYCQQWDTYPLTFGQG TKVEIK (SEQ ID NO: 55)

TABLE 13

KD values for Fab binding to human and rhesus CD122

| Analyte | VH Chain ID | Vk Chain ID | KD on hCD122 (nM) | KD on rhCD122 (nM) |
|---|---|---|---|---|
| FAB14 | F11VH-12_VH | F11VK-23_Vk | 4.34 | 3.52 |
| FAB13 | F11VH-12_VH | F11VK-12_Vk | 5.37 | 3.25 |
| FAB15 | F11VH-12_VH | F11VK-123_Vk | 5.54 | 4.47 |
| FAB06 | F11VH-2_VH | F11VK-123_Vk | 8.04 | 9.78 |
| FAB16 | F11VH-123_VH | F11VK-12_Vk | 8.56 | 5.04 |
| FAB05 | F11VH-2_VH | F11VK-23_Vk | 10.18 | 8.86 |
| FAB17 | F11VH-123_VH | F11VK-23_Vk | 10.67 | 7.93 |
| FAB18 | F11VH-123_VH | F11VK-123_Vk | 11.46 | 8.55 |
| VillFAb-1 | Vill_1_VH_Fab CH | Vill_1_VL | 12.42 | 10.18 |
| FAB04 | F11VH-2_VH | F11VK-12_Vk | 13.75 | 9.26 |
| FAB06F11-V | MAB06F11_VH | MAB06F11-V_VK | 15.15 | 13.86 |
| FAB11 | F11VH-23_VH 3 | F11VK-23_Vk | 17.67 | 15.87 |
| FAB10 | F11VH-23_VH | F11VK-12_Vk | 20.99 | 16.72 |
| FAB12 | F11VH-23_VH | F11VK-123_Vk | 21.83 | 19.51 |
| FAB07 | F11VH-13_VH | F11VK-12_Vk | 30.08 | 16.27 |
| FAB01 | F11VH-1_VH | F11VK-12_Vk | 33.64 | 14.38 |
| FAB03 | F11VH-1_VH | F11VK-123_Vk | 38.58 | 20.55 |
| FAB08 | F11VH-13_VH | F11VK-23_Vk | 41.25 | 30.68 |
| FAB02 | F11VH-1_VH | F11VK-23_Vk | 50.61 | 22.5 |
| FAB09 | F11VH-13_VH | F11VK-123_Vk | 72.5 | 36.52 |

TABLE 14

In vitro potency of selected clones

| CLONE | Inhibition of IL-15 Mediated M07e Proliferation | Biacore: Fab Affinity and Kinetics hCD122 | | | Non-Specific Binding | | |
|---|---|---|---|---|---|---|---|
| | IC₅₀ (mg/mL) | Ka (⅟Ms) | Kd (⅟s) | K₀ (nM) | BCAM | NEUDESIN | CILP2 |
| VillFAb-1 | 12.1 | 2.7E+05 | 3.4E-03 | 12.4 | strong | -- | -- |
| 06F11-V | 14.7 | 1.7E+06 | 2.6E-02 | 15.2 | -- | Strong | medium |
| FAB05 | 5.2 | 5.7E+05 | 5.8E-03 | 10.2 | -- | -- | -- |
| FAB06 | 7.6 | 5.4E+05 | 4.4E-03 | 8.0 | -- | -- | -- |
| FAB14 | 4.9 | 8.5E+05 | 3.7E-03 | 4.3 | strong | -- | -- |
| FAB15 | 5.1 | 6.1 E+05 | 3.4E-03 | 5.5 | strong | -- | -- |
| FAB17 | 9.7 | 4.3E+05 | 4.6E-03 | 10.7 | strong | -- | -- |
| FAB18 | 11.1 | 3.4E+05 | 3.9E-03 | 11.5 | medium | -- | -- |

BCAM = basal cell adhesion molecule
NEUDESIN = neudesin neutrophic factor
CILP2 = cartilage intermediate layer protein 2
-- = No measurable binding

TABLE 15

Examples of antibody Fc region amino acid sequences
Human IgG4 wild type
ASTKGPSVFPLAPCSRSTSESTAALGCLVKDYFPEPVTVSWNSGALTSGV HTFPAVLQSSGLYSLSSVVT
VPSSSLGTKTYTCNVDHKPSNTKVDKRVESKYGPPCPSCPAPEFLGGPSV FLFPPKPKDTLMISRTPEVT
CVVVDVSQED PEVQFNWYVDGVEVHNAKTKPREEQFNSTYRVVSVLTVLHQDWLNGKEYK CKVSNKGLP
SSIEKTISKAKGQPREPQVYTLPPSQEEMTKNQVSLTCLVKGFYPSDIAV EWESNGQPENNYKTTPPVLD
SDGSFFLYSRLTVDKSRWQEGNVFSCSVMHEALHNHYTQKSLSLSLGK
(SEQ ID NO: 32)

Human IgG4(S228P)
ASTKGPSVFPLAPCSRSTSESTAALGCLVKDYFPEPVTVSWNSGALTSGV HTFPAVLQSSGLYSLSSVVT
VPSSSLGTKTYTCNVDHKPSNTKVDKRVESKYGPPCPPCPAPEFLGGPSV FLFPPKPKDTLMISRTPEVT
CVVVDVSQED PEVQFNWYVDGVEVHNAKTKPREEQFNSTYRVVSVLTVLHQDWLNGKEYK CKVSNKGLP
SSIEKTISKAKGQPREPQVYTLPPSQEEMTKNQVSLTCLVKGFYPSDIAV EWESNGQPENNYKTTPPVLD
SDGSFFLYSRLTVDKSRWQEGNVFSCSVMHEALHNHYTQKSLSLSLGK
(SEQ ID NO: 33)

Human IgG1 wild type
ASTKGPSVFPLAPSSKSTSGGTAALGCLVKDYFPEPVTVSWNSGALTSGV HTFPAVLQSSGLYSLSSVVT
VPSSSLGTQTYICNVNHKPSNTKVDKKVEPKSCDKTHTCPPCPAPELLGG PSVFLFPPKPKDTLMISRTP
EVTCVVVDVSHEDPEVKFNWYVDGVEVHNAKTKPREEQYNSTYRVVSVLT VLHQDWLNGKEYKCKVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
(SEQ ID NO: 34)

Human IgG1-3M
ASTKGPSVFPLAPSSKSTSGGTAALGCLVKDYFPEPVTVSWNSGALTSGV HTFPAVLQSSGLYSLSSVVT
VPSSSLGTQTYICNVNHKPSNTKVDKKVEPKSCDKTHTCPPCPAPEAAGA PSVFLFPPKPKDTLMISRTP
EVTCVVVDVSHEDPEVKFNWYVDGVEVHNAKTKPREEQYNSTYRVVSVLT VLHQDWLNGKEYKCKVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
(SEQ ID NO: 35)

Human IgG2 wild type
ASTKGPSVFPLAPCSRSTSESTAALGCLVKDYFPEPVTVSWNSGALTSGV HTFPAVLQSSGLYSLSSVVT
VPSSNFGTQTYTCNVDHKPSNTKVDKTVERKCCVECPPCPAPPVAGPSVF LFPPKPKDTLMISRTPEVTC
VVVDVSHEDPEVQFNWYVDGVEVHNAKTKPREEQFNSTFRVVSVLTVVHQ DWLNGKEYKCKVSNKGLPAP
IEKTISKTKGQPREPQVYTLPPSREEMTKNQVSLTCLVKGFYPSDISVEW ESNGQPENNYKTTPPMLDSD
GSFFLYSKLTVDKSRWQQGNVFSCSVMHEALHNHYTQKSLSLSPGK
(SEQ ID NO: 36)

Human IgG1 wild type “REEM” allotype
ASTKGPSVFPLAPSSKSTSGGTAALGCLVKDYFPEPVTVSWNSGALTSGV HTFPAVLQSSGLYSLSSVVT
VPSSSLGTQTYICNVNHKPSNTKVDKKVEPKSCDKTHTCPPCPAPELLGG PSVFLFPPKPKDTLMISRTP
EVTCVVVDVSHEDPEVKFNWYVDGVEVHNAKTKPREEQYNSTYRVVSVLT VLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
(SEQ ID NO: 37)

Human IgG1-3M “REEM” allotype
ASTKGPSVFPLAPSSKSTSGGTAALGCLVKDYFPEPVTVSWNSGALTSGV HTFPAVLQSSGLYSLSSVVT
VPSSSLGTQTYICNVNHKPSNTKVDKKVEPKSCDKTHTCPPCPAPEAAGA PSVFLFPPKPKDTLMISRTP
EVTCVVVDVSHEDPEVKFNWYVDGVEVHNAKTKPREEQYNSTYRVVSVLT VLHQDWLNGKEYKCKVSNKA
LPAPIEKTISKAKGQPREPQVYTLPPSREEMKNQVSLTCLVKGFYPSDIAVEWESNGQPENNYKTTPPVL
DSDGSFFLYSKLTVDKSRWQQGNVFSCSVMHEALHNHYTQKSLSLSPGK
(SEQ ID NO: 38)

TABLE 16

Examples of membrane protein amino acid sequences
Human CD122 sequence
MAAPALSWRLPLLILLLPLATSWASAAVNGTSQFTCFYNSRANISCVWSQ DGALQDTSCQVHAWPDRRRW
NQTCELLPVSQASWACNLILGAPDSQKLTTVDIVTLRVLCREGVRWRVMA IQDFKPFENLRLMAPISLQV
VHVETHRCNISWEISQASHYFERHLEFEARTLSPGHTWEEAPLLTLKQKQ EWICLETLTPDTQYEFQVRV
KPLQGEFTTWSPWSQPLAFRTKPAALGKDTIPWLGHLLVGLSGAFGFIIL VYLLINCRNTGPWLKKVLKC
NTPDPSKFFSQLSSEHGGDVQKWLSSPFPSSSFSPGGLAPEISPLEVLER DKVTQLLLQQDKVPEPASLS
SNHSLTSCFTNQGYFFFHLPDALEIEACQVYFTYDPYSEEDPDEGVAGAP TGSSPQPLQPLSGEDDAYCT
FPSRDDLLLFSPSLLGGPSPPSTAPGGSGAGEERMPPSLQERVPRDWDPQ PLGPPTPGVPDLVDFQPPPE
LVLREAGEEVPDAGPREGVSFPWSRPPGQGEFRALNARLPLNTDAYLSLQ ELQGQDPTHLV
(SEQ ID NO: 19)

Cynomolgus and rhesus monkey CD122 sequence
MATLALSWCLPLLILLLPLATSSASAAVNGTSRFTCFYNSRANISCVWSQ DGALQDTSCQVHAWPDRRRW
NQTCELLPVSQASWACNLILGTPDSQKLTAVDIVTLRVMCREGVRWRMMA IQDFKPFENLRLMAPISLQV
VHVETHRCNISWKISQASHYFERHLEFEARTLSPGHTWEEAPLMTLKQKQ EWICLETLTPDTQYEFQVRV
KPLQGEFTTWSPWSQPLAFRTKPAALGKDTIPWLGHLLVGLSGAFGFIIL VYLLINCRNTGPWLKKVLKC
HTPDPSKFFSQLTSEHGGDVQKWLSSPFPSSSFSPGGLAPEISPLEVLER DKVTQLLLQQDKVPEPSSLS
SNRSLTSCFTNQGYFFFHLPDALEIEACQVYFTYDPCAEEEPDEGGADAP TGSSPQPLRPLSAEDDAYCT
FPSGDDLLLFSPSLLGGPSPPSTAPGGSGAGEERLPPSLQERVPRDWDPQ PLGPPTPGVPDLVDFQPRPE
LVLREAGEQVPDPGPREPFSFPWARPPGQGEVRALNARLPLNTDAYLSLQ ELQDQDPTHLV
(SEQ ID NO: 20)

Human BCAM sequence
MEPPDAPAQARGAPRLLLLAVLLAAHPDAQAEVRLSVPPLVEVMRGKSVI LDCTPTGTHDHYMLEWFLTD
RSGARPRLASAEMQGSELQVTMHDTRGRSPPYQLDSQGRLVLAEAQVGDE RDYVCVVRAGAAGTAEATAR
LNVFAKPEATEVSPNKGTLSVMEDSAQEIATCNSRNGNPAPKITWYRNGQ RLEVPVEMNPEGYMTSRTVR
EASGLLSLTSTLYLRLRKDDRDASFHCAAHYSLPEGRHGRLDSPTFHLTL HYPTEHVQFWVGSPSTPAGW
VREGDTVQLLCRGDGSPSPEYTLFRLQDEQEEVLNVNLEGNLTLEGVTRG QSGTYGCRVEDYDAADDVQL
SKTLELRVAYLDPLELSEGKVLSLPLNSSAVVNCSVHGLPTPALRWTKDS TPLGDGPMLSLSSITFDSNG
TYVCEASLPTVPVLSRTQNFTLLVQGSPELKTAEIEPKADGSWREGDEVT LICSARGHPDPKLSWSQLGG
SPAEPIPGRQGWVSSSLTLKVTSALSRDGISCEASNPHGNKRHVFHFGTV SPQTSQAGVAVMAVAVSVGL
LLLVVAVFYCVRRKGGPCCRQRREKGAPPPGEPGLSHSGSEQPEQTGLLM GGASGGARGGSGGFGDEC
(SEQ ID NO: 21)

TABLE 17

BIACORE® analysis of Fc receptor interactions

| | | | | | | |
|---|---|---|---|---|---|---|
| Receptor | Irrelevant IgG1 | Irrelevant IgG4 | 06F11-V IgG1-3M | VillMAb-1 IgG1 | Irrelevant mIgG1 | Irrelevant mIgG2a |
| hFcgRIIIA_{176F} | ++ | +/- | - | ++ | ND | ND |
| hFcgRIIIA_{176F} | +++ | - | - | +++ | ND | ND |
| hFcgRIIIB | ++ | +/- | - | ++ | ND | ND |
| hFcgRIIA_{167R} | ++ | + | +/- | ++ | ND | ND |
| hFcgRIIA_{167H} | ++ | + | - | ++ | ND | ND |
| hFcgRIIB | ++ | ++ | +/- | + | ND | ND |
| hFcgRI | ++++ | ++++ | - | ++++ | ND | ND |
| hFcRn pH 6.0 | ++ | ++ | ++ | ++ | - | - |
| hFcRn pH 7.4 | - | - | - | - | - | - |
| mFcγRI | ++++ | ++++ | - | ++++ | - | ++++ |
| mFcγRIII | ++ | + | - | ++ | ++ | ++ |
| mFcγRIV | ++ | +/- | - | ++ | - | +++ |
| mFcγRIIB | ++ | + | - | ++ | ++ | ++ |
| mFcRn pH 6.0 | ++ | ++ | ++ | ++ | +++ | +++ |
| mFcRn pH 7.4 | - | - | - | - | - | - |

+ = relative binding observed
- = no binding observed
ND = not done

TABLE 18

Amino acid sequences of antibody MAB05

| Antibody name | Mab5 | |
|---|---|---|
| Domain or Region | Sequence | SEQ ID NO |
| Heavy chain variable region | EVQLLESGGGLVQPGGSLRLSCAASGFTVTSYAVHWVRQAPG KGLEWLGVIWSGGSTDYNAAFISRLTISKDNSKNTVYFQMNS LRAEDTAVYYCARAGDANYDGFAYWGQGTLVTVSS | 1 |
| Heavy chain FR1 | EVQLLESGGGLVQPGGSLRLSCAASGFTVT | 2 |
| Heavy chain CDR1 | SYAVH | 3 |
| Heavy chain FR2 | WVRQAPGKGLEWLG | 4 |
| Heavy chain CDR2 | VIWSGGSTDYNAAFIS | 5 |
| Heavy chain FR3 | RLTISKDNSKNTVYFQMNSLRAEDTAVYYCAR | 6 |
| Heavy chain CDR3 | AGDANYDGFAY | 7 |
| Heavy chain FR4 | WGQGTLVTVSS | 8 |
| Light chain variable region | DIQMTQSPSSLSASVGDRVTITCQASQSVSFLYWYQQRPGKA PRLLIYDTSNLASGVPSRFSGSGSGTSYTFTISSLQPEDIAT YYCQQWSTYPLTFGQGTKVEIK | 9 |
| Light chain FR1 | DIQMTQSPSSLSASVGDRVTITC | 10 |
| Light chain CDR1 | QASQSVSFLY | 11 |
| Light chain FR2 | WYQQRPGKAPRLLIY | 12 |
| Light chain CDR2 | DTSNLAS | 13 |
| Light chain FR3 | GVPSRFSGSGSGTSYTFTISSLQPEDIATYYC | 14 |
| Light chain CDR3 | QQWSTYPLT | 15 |
| Light chain FR4 | FGQGTKVEIK | 16 |

CDR sequences are underlined in variable region sequences.

TABLE 19

Amino acid sequences of antibody MAB06

| Antibody name | Mab6 | |
|---|---|---|
| Domain or Region | Sequence | SEQ ID NO |
| Heavy chain variable region | EVQLLESGGGLVQPGGSLRLSCAASGFTVTSYAVHWVRQAPG KGLEWLGVIWSGGSTDYNAAFISRLTISKDNSKNTVYFQMNS LRAEDTAVYYCARAGDANYDGFAYWGQGTLVTVSS | 1 |
| Heavy chain FR1 | EVQLLESGGGLVQPGGSLRLSCAASGFTVT | 2 |
| Heavy chain CDR1 | SYAVH | 3 |
| Heavy chain FR2 | WVRQAPGKGLEWLG | 4 |
| Heavy chain CDR2 | VIWSGGSTDYNAAFIS | 5 |
| Heavy chain FR3 | RLTISKDNSKNTVYFQMNSLRAEDTAVYYCAR | 6 |
| Heavy chain CDR3 | AGDANYDGFAY | 7 |
| Heavy chain FR4 | WGQGTLVTVSS | 8 |
| Light chain variable region | DIQMTQSPSSLSASVGDRVTITCQASSSVSFMYWYQQRPGKA PRLLIYDTSNLASGVPSRFSGSGSGTSYTFTISSLQPEDIAT YYCQQWSTYPLTFGQGTKVEIK | 17 |
| Light chain FR1 | DIQMTQSPSSLSASVGDRVTITC | 10 |
| Light chain CDR1 | QASSSVSFMY | 18 |
| Light chain FR2 | WYQQRPGKAPRLLIY | 12 |
| Light chain CDR2 | DTSNLAS | 13 |
| Light chain FR3 | GVPSRFSGSGSGTSYTFTISSLQPEDIATYYC | 14 |
| Light chain CDR3 | QQWSTYPLT | 15 |
| Light chain FR4 | FGQGTKVEIK | 16 |

CDR sequences are underlined in variable region sequences.

TABLE 20

Amino acid sequences of murine/humanized antibody MIKβ1

| Antibody name | MIKβ1 | |
|---|---|---|
| Domain or Region | Sequence | SEQ ID NO |
| Heavy chain variable region | EVQLLESGGGLVQPGGSLRLSCAASGFSVTSYGVHWIRQAPG KGLEWLGVIWSGGSTDYNAAFISRLTISKDNSKNTVYFQMNS LQAEDTAIYYCARAGDYNYDGFAYWGQGTLVTVSS | 22 |
| Heavy chain FR1 | EVQLLESGGGLVQPGGSLRLSCAASGFSVT | 23 |
| Heavy chain CDR1 | SYGVH | 24 |
| Heavy chain FR2 | WIRQAPGKGLEWLG | 25 |
| Heavy chain CDR2 | VIWSGGSTDYNAAFIS | 5 |
| Heavy chain FR3 | RLTISKDNSKNTVYFQMNSLQAEDTAIYYCAR | 26 |
| Heavy chain CDR3 | AGDYNYDGFAY | 27 |
| Heavy chain FR4 | WGQGTLVTVSS | 8 |
| Light chain variable region | DIVLTQSPSSLSASVGDRVTITCSGSSSVSFMYWYQQRPGKA PRLLIYDTSNLASGVPSRFSGSGSGTSYTFTISSLQPEDIAT YYCQQWSTYPLTFGQGTKVEVK | 28 |
| Light chain FR1 | DIVLTQSPSSLSASVGDRVTITC | 29 |
| Light chain CDR1 | SGSSSVSFMY | 30 |
| Light chain FR2 | WYQQRPGKAPRLLIY | 12 |
| Light chain CDR2 | DTSNLAS | 13 |
| Light chain FR3 | GVPSRFSGSGSGTSYTFTISSLQPEDIATYYC | 14 |
| Light chain CDR3 | QQWSTYPLT | 15 |
| Light chain FR4 | FGQGTKVEVK | 31 |

CDR sequences are underlined in variable region sequences.

What is claimed is:

1. An anti-CD122 antibody or an antigen-binding portion thereof, wherein the antibody or antigen-binding portion comprises a heavy chain variable (VH) region and a light chain variable (VL) region, wherein the VH region amino acid sequence comprises a HCDR1 comprising SEQ ID NO: 3, a HCDR2 comprising SEQ ID NO: 5 and a HCDR3 comprising SEQ ID NO: 7; and the VL region amino acid sequence comprises a LCDR1 comprising SEQ ID NO: 11, a LCDR2 comprising SEQ ID NO: 13 and a LCDR3 comprising SEQ ID NO: 15.

2. The antibody or antigen-binding portion of claim 1, wherein the VH region amino acid sequence comprises SEQ ID NO: 1 and the VL region amino acid sequence comprises SEQ ID NO: 9.

3. The antibody or antigen-binding portion of claim 1, wherein the antibody or antigen-binding portion is humanized or chimeric.

4. The antibody or antigen-binding portion of claim 1, wherein the VH region, the VL region, or both the VH and the VL region comprise one or more human framework region amino acid sequences.

5. The antibody or antigen-binding portion of claim 1, wherein the VH region, the VL region, or both the VH and the VL region comprise a human variable region framework scaffold amino acid sequence into which the CDR amino acid sequences have been inserted.

6. The antibody or antigen-binding portion of claim 1, wherein the VH region comprises an IGHV3-23 human germline scaffold amino acid sequence into which the HCDR1, HCDR2 and HCDR3 amino acid sequences have been inserted.

7. The antibody or antigen-binding portion of claim 1, wherein the VL region comprises an IGKV1-33 human germline scaffold amino acid sequence into which the LCDR1, LCDR2 and LCDR3 amino acid sequences have been inserted.

8. The antibody or antigen-binding portion of claim 1, wherein the antibody or antigen-binding portion comprises an immunoglobulin constant region.

9. The antibody or antigen-binding portion of claim 8, wherein the immunoglobulin constant region is IgG, IgE, IgM, IgD, IgA or IgY.

10. The antibody or antigen-binding portion of claim 9, wherein the immunoglobulin constant region is IgG1, IgG2, IgG3, IgG4, IgA1 or IgA2.

11. The antibody or antigen-binding portion of claim 8, wherein the immunoglobulin constant region is immunologically inert.

12. The antibody or antigen-binding portion of claim 8, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A, L235A and G237A or a wild-type human IgG2 constant region, wherein numbering is according to the EU index as in Kabat.

13. The antibody or antigen-binding portion of claim 8, wherein the immunoglobulin constant region comprises any one of SEQ ID NOs: 32-38.

14. The antibody or antigen-binding portion of claim 1, wherein the antibody or antigen-binding portion is an Fab, an Fab′, an F(ab′)₂, an Fv, an scFv, a maxibody, a minibody, a diabody, a triabody, a tetrabody, or a bis-scFv.

15. The antibody or antigen-binding portion of claim 1, wherein the antibody or antigen-binding portion is monoclonal.

16. The antibody or antigen-binding portion of claim 1, wherein the antibody or antigen-binding portion is tetrameric, tetravalent or multispecific.

17. The antibody or antigen-binding portion of claim 1, wherein the antibody or antigen-binding portion is a bispecific antibody or a bispecific antigen-binding portion that binds specifically to a first antigen and a second antigen, wherein the first antigen is CD122 and the second antigen is not CD122.

18. An immunoconjugate comprising the antibody or antigen-binding portion of claim 1, linked to a therapeutic agent.

19. The immunoconjugate of claim 18, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

20. A pharmaceutical composition comprising the antibody or antigen-binding portion of claim 1, and a pharmaceutically acceptable carrier, diluent or excipient.

21. A nucleic acid molecule encoding
(a) the VH region amino acid sequence;
(b) the VL region amino acid sequence; or
(c) both the VH and the VL region amino acid sequences of the antibody or antigen-binding portion of claim 1.

22. An expression vector comprising the nucleic acid molecule of claim 21.

23. A recombinant host cell comprising the nucleic acid molecule of claim 21.

24. A method of producing an anti-CD122 antibody or an antigen-binding portion thereof, the method comprising:
culturing the recombinant host cell of claim 23 under conditions whereby the nucleic acid molecule is expressed, thereby producing the antibody or antigen-binding portion; and
isolating the antibody or antigen-binding portion from the host cell or culture.

25. A method for supressing an immune response in a subject, comprising administering to the subject a therapeutically effective amount of the antibody or antigen-binding portion of claim 1.

26. The method of claim 25, wherein the immune response is mediated by CD122.

27. A method for treating or preventing a disease in a subject, comprising administering to the subject a therapeutically effective amount of the antibody or antigen-binding portion of claim 1.

28. The method of claim 27, wherein the disease is an inflammatory disease or an autoimmune disease.

29. The method of claim 27, wherein the disease is vitiligo, celiac disease, type 1 diabetes, multiple sclerosis, graft-versus-host disease, systemic lupus erythematosus, psoriasis, atopic dermatitis, alopecia areata, ulcerative colitis, or rheumatoid arthritis.

30. The method of claim 27, wherein the disease is vitiligo.

31. The method of claim 27, wherein the administration to the subject is via intravenous infusion.

32. The method of claim 27, wherein the administration to the subject is subcutaneous.

33. A method for supressing IL-15 induced migration of T cells from skin, the method comprising contacting the skin with a therapeutically effective amount of the antibody or antigen-binding portion of claim 1.
